SEQUENCE LISTING (141 sequences)

SEQ ID NO: 1 - DNA, 509 bp, Homo Sapiens
tgggtcctgtcccaggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtttatctattacactgggagcaccaactacaacccctccctcaagagtcgagtctccatatcagtagacacgtccaagaaccagttctccctgaagctgagctctgtgaccgctgcggacgcggccgtgtattactgtgcgagagattatgactggagcttccactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctct

SEQ ID NO: 2 - protein, 121 aa, Homo Sapiens
QVQLQESGPGLVKPSETLSLTCTVSGGSVSSGGYYWSWIRQPPGKGLEWIGFIYYTGSTNYNPSLKSRVSISVDTSKNQFSLKLSSVTAADAAVYYCARDYDWSFHFDYWGQGTLVTVSSA

SEQ ID NO: 3 - DNA, 504 bp, Homo Sapiens
cagctcctggggctcctgctgctctggttcccaggtgccaggtgtgacatccagatgacccagtctccatcctccctgtctgcatctataggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcc

SEQ ID NO: 4 - protein, 108 aa, Homo Sapiens
DIQMTQSPSSLSASIGDRVTITCRASQGIRNDLGWYQQKPGKAPKRLIYAASSLQSGVPSRFSGSGSGTEFTLTISSLQPEDFATYYCLQHNSYPLTFGGGTKVEIKR

SEQ ID NO: 5 - DNA, 469 bp, Homo Sapiens
cagtgtgaggtgcagctggtggagtctgggggaggcttggtccagcctggggggtccctgagactctcctgtgcagcctctggattcacctttactaactattggatgagctgggtccgccaggctccagggaaggggctggagtgggtggccaacatacagcaagatggaagtgagaaatactatgtggactctgtgaggggccgattcaccatctccagagacaacgccaagaactcactgtatctgcaaatgaacagcctgagagccgaggactcggctgtgtattactgtgcgagatgggactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgagcggtgtcgtggaac

SEQ ID NO: 6 - protein, 113 aa, Homo Sapiens
EVQLVESGGGLVQPGGSLRLSCAASGFTFTNYWMSWVRQAPGKGLEWVANIQQDGSEKYYVDSVRGRFTISRDNAKNSLYLQMNSLRAEDSAVYYCARWDYWGQGTLVTVSSA

SEQ ID NO: 7 - DNA, 454 bp, Homo Sapiens
cttctggggctgctaatgctctgggtccctggatccagtggggatattgtgatgacccagactccactctcctcaactgtcatccttggacagccggcctccatctcctgcaggtctagtcaaagcctcgtacacagtgatggaaacacctacttgaattggcttcagcagaggccaggccagcctccaagactcctaatttatatgatttctaaccggttctctggggtcccagacagattcagtggcagtggggcagggacagatttcacactgaaaatcagcagggtggaagctgaggatgtcggggtttattactgcatgcaagctacagaatctcctcagacgttcggccaagggaccaaggtggaaatcaaacgaactgtggctgcaccatctgtcttcatcttcccgccatctgatgagcagttgaaatctggaagggcctctgttg

SEQ ID NO: 8 - protein, 113 aa, Homo Sapiens
DIVMTQTPLSSTVILGQPASISCRSSQSLVHSDGNTYLNWLQQRPGQPPRLLIYMISNRFSGVPDRFSGSGAGTDFTLKISRVEAEDVGVYYCMQATESPQTFGQGTKVEIKR

SEQ ID NO: 9 - DNA, 529 bp, Homo Sapiens
gagcagtcggggggaggcgtggtaaagcctggggggtctcttagactctcctgtgcagcctctggattcactttcagtaacgcctggatgacctgggtccgccaggctccagggaaggggctggagtgggttggccgtattaaaaggagaactgatggtgggacaacagactacgctgcacccgtgaaaggcagattcaccatctcaagagatgattcaaaaaacacgctgtatctgcaaatgaacaacctgaaaaacgaggacacagccgtgtattactgtacctcagtcgataatgacgtggactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactct

SEQ ID NO: 10 - protein, 119 aa, Homo Sapiens
NNNNEQSGGGVVKPGGSLRLSCAASGFTFSNAWMTWVRQAPGKGLEWVGRIKRRTDGGTTDYAAPVKGRFTISRDDSKNTLYLQMNNLKNEDTAVYYCTSVDNDVDYWGQGTLVTVSSA

SEQ ID NO: 11 - DNA, 447 bp, Homo Sapiens
ctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatattggtctttattactgcatgcaagctctacaaactccgctcactttcggcggagggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacag

SEQ ID NO: 12 - protein, 113 aa, Homo Sapiens
NNNLTQSPLSLPVTPGEPASISCRSSQSLLHSNGYNYLDWYLQKPGQSPQLLIYLGSNRASGVPDRFSGSGSGTDFTLKISRVEAEDIGLYYCMQALQTPLTFGGGTKVDIKR

SEQ ID NO: 13 - DNA, 538 bp, Homo Sapiens
caggtgcagctggagcagtcggggggaggcttggtacagcctggggggtccctgagactctcctgtgcagcctctggattcaccttcagtacctatagcatgaactgggtccgccaggctccagggaaggggctggagtgggtttcatacattagaagtagtactagtaccatatactatgcagagtccctgaagggccgattcaccatctccagcgacaatgccaagaattcactatatctgcaaatgaacagcctgagagacgaggacacggctgtgtattactgtgcgcgggactttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagca

SEQ ID NO: 14 - protein, 114 aa, Homo Sapiens
QVQLEQSGGGLVQPGGSLRLSCAASGFTFSTYSMNWVRQAPGKGLEWVSYIRSSTSTIYYAESLKGRFTISSDNAKNSLYLQMNSLRDEDTAVYYCARDFDYWGQGTLVTVSSA

SEQ ID NO: 15 - DNA, 490 bp, Homo Sapiens
gaaatccagctgactcagtctccactctcctcacctgtcacccttggacagccggcctccatctcctgcaggtctagtcaaagcctcgtacacagtgatggagacacctacttgaattggcttcagcagaggccaggccagcctccaagactcctaatttataagatttctacccggttctctggggtccctgacagattcagtggcagtggggcagggacagatttcacactgaaaatcagcagggtggagactgacgatgtcgggatttattactgcatgcaaactacacaaattcctcaaatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggta

SEQ ID NO: 16 - protein, 114 aa, Homo Sapiens
EIQLTQSPLSSPVTLGQPASISCRSSQSLVHSDGDTYLNWLQQRPGQPPRLLIYKISTRFSGVPDRFSGSGAGTDFTLKISRVETDDVGIYYCMQTTQIPQITFGQGTRLEIKR

SEQ ID NO: 17 - DNA, 568 bp, Homo Sapiens
caggtgcagctggagcagtcggggggaggcgtggtccagcctgggaggtccctgagactctcctgtgcagcgtctggattcaccttcagtcgctatggcatgcactgggtccgccaggctccaggcaaggggctgaaatgggtggcagttatatggtatgatggaagtaataaactctatgcagactccgtgaagggccgattcaccatctccagagacaattccaagaacacgctgtatctgcaaatgaacagcctgagagccgaggacacggctgtgtattactgtgcgagagattactatgataatagtagacatcactgggggtttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagca

SEQ ID NO: 18 - protein, 124 aa, Homo Sapiens
QVQLEQSGGGVVQPGRSLRLSCAASGFTFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSA

SEQ ID NO: 19 - DNA, 472 bp, Homo Sapiens
gacatccagctgacccagtctccatcctccctgtctgcatctgtaggagacagagtcaccatcacttgccgggcaagtcagagtatttatagttatttaaattggtatcagcagaaaccagggaaagcccctaagctcctgatctatgctgcatccagtttgcaaagtggggtcccatccaggttcagtggcagtggatctgggacagatttcactctcaccatcagcagtctgcaacctgaagattttgcaacttactactgtcaacagagttacagtacccctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggta

SEQ ID NO: 20 - protein, 108 aa, Homo Sapiens
DIQLTQSPSSLSASVGDRVTITCRASQSIYSYLNWYQQKPGKAPKLLIYAASSLQSGVPSRFSGSGSGTDFTLTISSLQPEDFATYYCQQSYSTPPTFGQGTKVEIKR

SEQ ID NO: 21 - DNA, 528 bp, Homo Sapiens
cagtcggggggaggcttggtaaagcctggggggtcccttagactctcctgtgcagcctctggattcactttcagtaacgcctggatgacctgggtccgccaggctccagggaaggggctggagtgggttggccgtattaaaaggaaaactgatggtgggacaacagactacgctgcacccgtgaaaggcagattcaccatctcaagagatgattcagaaaacacgctgtatctgcaaatgaacagcctggaaaccgaggacacagccgtgtattactgtaccacagtcgataacagtggtgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctct

SEQ ID NO: 22 - protein, 119 aa, Homo Sapiens
NNNNNQSGGGLVKPGGSLRLSCAASGFTFSNAWMTWVRQAPGKGLEWVGRIKRKTDGGTTDYAAPVKGRFTISRDDSENTLYLQMNSLETEDTAVYYCTTVDNSGDYWGQGTLVTVSSA

SEQ ID NO: 23 - DNA, 466 bp, Homo Sapiens
actcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctca

SEQ ID NO: 24 - protein, 113 aa, Homo Sapiens
NNNNTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKR

SEQ ID NO: 25 - DNA, 537 bp, Homo Sapiens
caggtgcagctggagcagtcggggggaggcgtggtccagcctgggaggtccctgagactctcctgtgcagcgtctggattcaccttcactaactatggcttgcactgggtccgccaggctccaggcaaggggctggattgggtggcagttatatggtatgatggaagtcataaattctatgcagactccgtgaagggccgattcaccatctccagagacaattccaagaacacgctctttctgcaaatgaacagcctgagagccgaggacacggctgtgtattactgtacgcgagatcttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagc

SEQ ID NO: 26 - protein, 114 aa, Homo Sapiens
QVQLEQSGGGVVQPGRSLRLSCAASGFTFTNYGLHWVRQAPGKGLDWVAVIWYDGSHKFYADSVKGRFTISRDNSKNTLFLQMNSLRAEDTAVYYCTRDLDYWGQGTLVTVSSA

SEQ ID NO: 27 - DNA, 480 bp, Homo Sapiens
gaaacgcagctgacgcagtctccaggcaccctgtctttgtctccaggggaaagagtcaccctctcctgcagggccagtcagagtgttagcaacaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattgtgcagagtgttactgtcagcaatatggtagctcactcccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtgggaaggtgggataacgccctccaatcgggta

SEQ ID NO: 28 - protein, 110 aa, Homo Sapiens
ETQLTQSPGTLSLSPGERVTLSCRASQSVSNNYLAWYQQKPGQAPRLLIYGASSRATGIPDRFSGSGSGTDFTLTISRLEPEDCAECYCQQYGSSLPLTFGGGTKVEIKR

SEQ ID NO: 29 - DNA, 542 bp, Homo Sapiens
gtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtcataaatactatgcagactccgtgaagggccgattcaccatctccagagacaattccaagaacacgctgtatctgcaaatgaacagcctgagagccgaggacacggctgtgtattactctgcgagagattactatgatacgagtcggcatcactgggggtttgactgctggggccagggaaccctggtcaccgtctcctctgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggc

SEQ ID NO: 30 - protein, 124 aa, Homo Sapiens
QVQLVESGGGVVQPGRSLRLSCAASGFTFSSYGMHWVRQAPGKGLEWVAVIWYDGSHKYYADSVKGRFTISRDNSKNTLYLQMNSLRAEDTAVYYSARDYYDTSRHHWGFDCWGQGTLVTVSSA

SEQ ID NO: 31 - DNA, 521 bp, Homo Sapiens
cagctcctggggctgctaatgctctgggtccctggatccagtgaggaaattgtgatgacccagactccactctccctgcccgtcacccctggagagccggcctccatctcctgcaggtctagtcagagcctcttggatagtgaagatggaaacacctatttggactggtacctgcagaagccagggcagtctccacagctcctgatctatacgctttcccatcgggcctctggagtcccagacaggttcagtggcagtgggtcaggcactgatttcacactgaaaatcagcagggtggaggctgaggatgttggagtttattgctgcatgcaacgtgtagagtttcctatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 32 - protein, 114 aa, Homo Sapiens
EIVMTQTPLSLPVTPGEPASISCRSSQSLLDSEDGNTYLDWYLQKPGQSPQLLIYTLSHRASGVPDRFSGSGSGTDFTLKISRVEAEDVGVYCCMQRVEFPITFGQGTRLEIKR

SEQ ID NO: 33 - DNA, 547 bp, Homo Sapiens
cagtcgggcccaagactggtgaagccttcacagaccctgtccctcacctgcactgtctctggtggctccatcagtagtgatggttactactggagctggatccgccagcacccagggaagggcctggagtggattgggtacatctattacagtgggagcaccttctacaacccgtccctcaagagtcgagttgccatatcagtggacacgtctaagaaccagttctccctgaagctgagctctgtgactgccgcggacacggccgtgtattactgtgcgagagaatcccctcatagcagcaactggtactcgggctttgactgctggggccagggaaccctggtcaccgtctcctcagcttccaccaagggcccatccgtcttccccctggcgccctgctccaggagcacctccgagagcacagccgccctgggctgcctggtcaaggactactttccccgaaccggtgacggtgtcgtggaactcaggcgccctgaccagcggcgtgcacaccttcccggctgtcctacagtcctcaggactctct

SEQ ID NO: 34 - protein, 125 aa, Homo Sapiens
NNNNNQSGPRLVKPSQTLSLTCTVSGGSISSDGYYWSWIRQHPGKGLEWIGYIYYSGSTFYNPSLKSRVAISVDTSKNQFSLKLSSVTAADTAVYYCARESPHSSNWYSGFDCWGQGTLVTVSSA

SEQ ID NO: 35 - DNA, 450 bp, Homo Sapiens
actcagtctccagactttcagtctgtgactccaaaggagaaagtcaccatcacctgccgggccagtcagagcattggtagtaggttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtaatttaccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctc

SEQ ID NO: 36 - protein, 108 aa, Homo Sapiens
NNNNTQSPDFQSVTPKEKVTITCRASQSIGSRLHWYQQKPDQSPKLLIKYASQSFSGVPSRFSGSGSGTDFTLTINSLEAEDAATYYCHQSSNLPFTFGPGTKVDIKR

SEQ ID NO: 37 - DNA, 534 bp, Homo Sapiens
caggtgcagctggtggaggctgggggaggcgtggtccagcctgggaggtccctgagactctcctgtgcagcgtctggattcaccttcagaagctatggcatgcactgggtccgccaggctccaggcaaggggctgaaatgggtggcagttatatggtatgatggaagtaataaatactatacagactccgtgaagggccgattcaccatctccagagacaattccaagaacacgctgtatctgcaaatgaacagcctgagagccgaggacacggctgtgtattactgtgtgagagattactatgataatagtagacatcactgggggtttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccaggcggcgtgcacaccttcccggc

SEQ ID NO: 38 - protein, 124 aa, Homo Sapiens
QVQLVEAGGGVVQPGRSLRLSCAASGFTFRSYGMHWVRQAPGKGLKWVAVIWYDGSNKYYTDSVKGRFTISRDNSKNTLYLQMNSLRAEDTAVYYCVRDYYDNSRHHWGFDYWGQGTLVTVSSA

SEQ ID NO: 39 - DNA, 470 bp, Homo Sapiens
gacatccagatgacccagtctccatcctcccggtgtgcatccgtaggagacagagtcaccatcacttgccgggcaagtcagggcatcagaaatgatttagcttggtatcagcagaaaccagggaaagcccctaagcgcctgatctatgctgcatccagtttgcaaagtggggtcccatcaaggttcagcggcagtagatctgggacagaattcactctcacaatcagcagcctgcagcctgaagattttgcagcttattactgtctccagcataatagttaccctcccagttttggccaggggaccaagctggagatcaaacgaactgtggctgcaccatctgtcttcatcttcccgccatctgatgagcagttgaaatctggaactgctagcgttgtgtgcctgctgaataacttctatcccagagaggccaaagtacagtggaaggtggataacgccctccaatcggg

SEQ ID NO: 40 - protein, 108 aa, Homo Sapiens
DIQMTQSPSSRCASVGDRVTITCRASQGIRNDLAWYQQKPGKAPKRLIYAASSLQSGVPSRFSGSRSGTEFTLTISSLQPEDFAAYYCLQHNSYPPSFGQGTKLEIKR

SEQ ID NO: 41 - DNA, 514 bp, Homo Sapiens
catgtgcaggtgcagctggtggagtctgggggaggcgtggtccagcctgggaggtccctgagactctcctgtgcagcgtctggattcatcttcagtcgctatggcatgcactgggtccgccaggctccaggcaaggggctgaaatgggtggcagttatatggtatgatggaagtaataaactctatgcagactccgtgaagggccgattcaccatctccagagacaattccaagaacacgctgtatctgcaaatgaacagcctgagagccgaggacacggctgtgtattactgtgcgagagattactatgataatagtagacatcactgggggtttgactactggggccagggaaccctggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctga

SEQ ID NO: 42 - protein, 124 aa, Homo Sapiens
QVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSA

SEQ ID NO: 43 - DNA, 523 bp, Homo Sapiens
tcagctcctggggctgctaatgctctgggtccctggatcagtgaggatattgtgatgacccagactccactctccctgcccgtcacccctggagagccggcctccatctcctgcaggtctagtcggagcctcttggatagtgatgatggaaacacctatttggactggtacctgcagaagccagggcagtctccacagctcctgatctacacgctttcctatcgggcctctggagtcccagacaggttcagtggcagtgggtcaggcactgatttcacactgaaaatcagcagggtggaggctgaggatgttggagtttattactgcatgcaacgtgtagagtttcctatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgcct

SEQ ID NO: 44 - protein, 114 aa, Homo Sapiens
DIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKR

SEQ ID NO: 45 - DNA, 546 bp, Homo Sapiens
gagcagtcggggggcggcgtggtccagcctgggaggtccctgagactctcctgtgcagcgtctggattcaccttcagtagctatggcatgtactgggtccgccaggctccaggcaaggggctggagtgggtggcagttatatggtatgatggaagcaataaatactatgcagactccgtgaagggccgattcaccatctccagagacaattccaagaacacgctgtatctgcaaatgaacagcctgagagccgaggacacggctgtgtattactgtgcgagggatttctatgatagtagtcgttaccactacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctct

SEQ ID NO: 46 - protein, 124 aa, Homo Sapiens
NNNNEQSGGGVVQPGRSLRLSCAASGFTFSSYGMYWVRQAPGKGLEWVAVIWYDGSNKYYADSVKGRFTISRDNSKNTLYLQMNSLRAEDTAVYYCARDFYDSSRYHYGMDVWGQGTTVTVSSA

SEQ ID NO: 47 - DNA, 419 bp, Homo Sapiens
actcagtgtccactctccctgcccgtcacccctggagagccggcctccatctcctgcaggtctagtcagagcctcttggatagtgatgatggaaacacctatttggactggtacctgcagaagccagggcagtctccacagctcctgatctatacggtttcctatcgggcctctggagtcccagacaggttcagtggcagtgggtcaggcactgatttcacactgaaaatcagcagggtggaggctgaggatgttggagtttattactgcatgcaacgtatagagtttccgatcaccttcggccaagggacccgactggagattaaacgaactgtggctgcaccatctgtcttcatcttcccgccatctgatgagcagttgaaatctggaactgcctctgttgtgtgcctgctgaataa

SEQ ID NO: 48 - protein, 114 aa, Homo Sapiens
NNNNTQCPLSLPVTPGEPASISCRSSQSLLDSDDGNTYLDWYLQKPGQSPQLLIYTVSYRASGVPDRFSGSGSGTDFTLKISRVEAEDVGVYYCMQRIEFPITFGQGTRLEIKR

SEQ ID NO: 49 - DNA, 789 bp, Homo Sapiens
tctgtaaaggttggtggagaggcaggtccatctgtcacactaccctgccactacagtggagctgtcacatcaatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaacgacaactgttccaacaacaatgagcattccaacgacaacgactgttccgacgacaatgactgtttcaacgacaacgagcgttccaacgacaacgagcattccaacaacaacaagtgttccagtgacaacaacggtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactg

SEQ ID NO: 50 - protein, 263 aa, Homo Sapiens
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVPTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLL

SEQ ID NO: 51 - protein, 114 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARNNDYWGQGTLVTVSSA

SEQ ID NO: 52 - protein, 124 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCANNNYDSSNNNYGMDVWGQGTTVTVSSA

SEQ ID NO: 53 - protein, 125 aa, Homo Sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARNNNNSSSWYNNFDYWGQGTLVTVSSA

SEQ ID NO: 54 - protein, 124 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDYYDSSNNNNNFDYWGQGTLVTVSSA

SEQ ID NO: 55 - protein, 119 aa, Homo Sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTNNDNNNDYWGQGTLVTVSSA

SEQ ID NO: 56 - protein, 121 aa, Homo Sapiens
QVQLQESGPGLVKPSETLSLTCTVSGGSVSSGGYYWSWIRQPPGKGLEWIGYIYYSGSTNYNPSLKSRVTISVDTSKNQFSLKLSSVTAADTAVYYCARNNNWNNNFDYWGQGTLVTVSSA

SEQ ID NO: 57 - protein, 119 aa, Homo Sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTNNNSGDYWGQGTLVTVSSA

SEQ ID NO: 58 - protein, 113 aa, Homo Sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARNDYWGQGTLVTVSSA

SEQ ID NO: 59 - protein, 114 aa, Homo Sapiens
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCANNFDYWGQGTLVTVSSA

SEQ ID NO: 60 - protein, 110 aa, Homo Sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSNNLTFGGGTKVEIKR

SEQ ID NO: 61 - protein, 113 aa, Homo Sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTNNTFGGGTKVEIKR

SEQ ID NO: 62 - protein, 108 aa, Homo Sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIKR

SEQ ID NO: 63 - protein, 114 aa, Homo Sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPNITFGQGTRLEIKR

SEQ ID NO: 64 - protein, 108 aa, Homo Sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKR

SEQ ID NO: 65 - protein, 113 aa, Homo Sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPQTFGQGTKVEIKR

SEQ ID NO: 66 - protein, 114 aa, Homo Sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPITFGQGTRLEIKR

SEQ ID NO: 67 - protein, 108 aa, Homo Sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPFTFGPGTKVDIKR

SEQ ID NO: 68 - protein, 108 aa, Homo Sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPNNFGQGTKLEIKR

SEQ ID NO: 69 - protein, 113 aa, Homo Sapiens; identical to SEQ ID NO: 65

SEQ ID NO: 70 - protein, 114 aa, Homo Sapiens; identical to SEQ ID NO: 66

SEQ ID NO: 71 - protein, 108 aa, Homo Sapiens; identical to SEQ ID NO: 67

SEQ ID NO: 72 - protein, 108 aa, Homo Sapiens
  misc_feature (96)..(96): Wherein Xaa may be any amino acid
  misc_feature (97)..(97): Wherein Xaa may be any amino acid
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPXXFGQGTKLEIKR

SEQ ID NO: 73 - DNA, 16 bp, Homo Sapiens
ttactatgataatagt

SEQ ID NO: 74 - DNA, 15 bp, Homo Sapiens
agacatcactggggg

SEQ ID NO: 75 - DNA, 17 bp, Homo Sapiens
atagcagcaactggtac

SEQ ID NO: 76 - DNA, 16 bp, Homo Sapiens
ttactatgataatagt

SEQ ID NO: 77 - DNA, 15 bp, Homo Sapiens
agacatcactggggg

SEQ ID NO: 78 - DNA, 16 bp, Homo Sapiens
ttactatgataatagt

SEQ ID NO: 79 - DNA, 15 bp, Homo Sapiens
agacatcactggggg

SEQ ID NO: 80 - DNA, 13 bp, Homo Sapiens
ctatgatagtagt

SEQ ID NO: 81 - DNA, 11 bp, Homo Sapiens
ttactatgata

SEQ ID NO: 82 - DNA, 20 bp, Homo Sapiens
cgagtcggcatcactggggg

SEQ ID NO: 83 - DNA, 22 bp, Homo Sapiens
caggtgcagctggagcagtcgg

SEQ ID NO: 84 - DNA, 24 bp, Homo Sapiens
gctgagggagtagagtcctgagga

SEQ ID NO: 85 - DNA, 19 bp, Homo Sapiens
cacaccgcggtcacatggc

SEQ ID NO: 86 - DNA, 20 bp, Homo Sapiens
ctactctagggcacctgtcc

SEQ ID NO: 87 - protein, 14 aa, Homo Sapiens
PMPLPRQNHEPVAT

SEQ ID NO: 88 - protein, 12 aa, Homo Sapiens
PMPLPRQNHEPV

SEQ ID NO: 89 - protein, 10 aa, Homo Sapiens
PMPLPRQNHE

SEQ ID NO: 90 - protein, 8 aa, Homo Sapiens
PMPLPRQN

SEQ ID NO: 91 - protein, 6 aa, Homo Sapiens
PMPLPR

SEQ ID NO: 92 - protein, 12 aa, Homo Sapiens
PLPRQNHEPVAT

SEQ ID NO: 93 - protein, 10 aa, Homo Sapiens
PRQNHEPVAT

SEQ ID NO: 94 - protein, 8 aa, Homo Sapiens
QNHEPVAT

SEQ ID NO: 95 - protein, 6 aa, Homo Sapiens
HEPVAT

SEQ ID NO: 96 - protein, 7 aa, Homo Sapiens
PLPRNHE

SEQ ID NO: 97 - protein, 6 aa, Homo Sapiens
LPRQNH

SEQ ID NO: 98 - protein, 10 aa, Homo Sapiens
PMPAPRQNHE

SEQ ID NO: 99 - protein, 10 aa, Homo Sapiens
PMPLARQNHE

SEQ ID NO: 100 - protein, 10 aa, Homo Sapiens
PMPLPAQNHE

SEQ ID NO: 101 - protein, 10 aa, Homo Sapiens
PMPLPRANHE

SEQ ID NO: 102 - protein, 10 aa, Homo Sapiens
PMPLPRQAHE

SEQ ID NO: 103 - protein, 10 aa, Homo Sapiens
PMPLPRQNAE

SEQ ID NO: 104 - protein, 8 aa, Homo Sapiens
PLPRQNHE

SEQ ID NO: 105 - protein, 7 aa, Homo Sapiens
LPRQNHE

SEQ ID NO: 106 - protein, 8 aa, Homo Sapiens
PLPRQNHE

SEQ ID NO: 107 - protein, 7 aa, Homo Sapiens
LPRQNHE

SEQ ID NO: 108 - DNA, 882 bp, Homo Sapiens
atgaaatacctgctgccgaccgctgctgctggtctgctgctcctcgctgcccagccggccatggccgatattgtgatgacccagactccactctccctgcccgtcacccctggagagccggcctccatctcctgcaggtctagtcggagcctcttggatagtgatgatggaaacacctatttggactggtacctgcagaagccagggcagtctccacagctcctgatctacacgctttcctatcgggcctctggagtcccagacaggttcagtggcagtgggtcaggcactgatttcacactgaaaatcagcagggtggaggctgaggatgttggagtttattactgcatgcaacgtgtagagtttcctatcaccttcggccaagggacacgactggagattaaactttccgcggacgatgcgaaaaaggatgctgcgaagaaagatgacgctaagaaagacgatgctaaaaaggacctccaggtgcagctggtggagtctgggggaggcgtggtccagcctgggaggtccctgagactctcctgtgcagcgtctggattcatcttcagtcgctatggcatgcactgggtccgccaggctccaggcaaggggctgaaatgggtggcagttatatggtatgatggaagtaataaactctatgcagactccgtgaagggccgattcaccatctccagagacaattccaagaacacgctgtatctgcaaatgaacagcctgagagccgaggacacggctgtgtattactgtgcgagagattactatgataatagtagacatcactgggggtttgactactggggccagggaaccctggtcaccgtctcctcagctagcgattataaggacgatgatgacaaatag

SEQ ID NO: 109 - protein, 271 aa, Homo Sapiens
DIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKLSADDAKKDAAKKDDAKKDDAKKDLQVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSASDYKDDDDK

SEQ ID NO: 110 - DNA, 1560 bp, Homo Sapiens
atggaaaccccagcgcagcttctcttcctcctgctactctggctcccagataccaccggagatattgtgatgacccagactccactctccctgcccgtcacccctggagagccggcctccatctcctgcaggtctagtcggagcctcttggatagtgatgatggaaacacctatttggactggtacctgcagaagccagggcagtctccacagctcctgatctacacgctttcctatcgggcctctggagtcccagacaggttcagtggcagtgggtcaggcactgatttcacactgaaaatcagcagggtggaggctgaggatgttggagtttattactgcatgcaacgtgtagagtttcctatcaccttcggccaagggacacgactggagattaaaggtggtggtggttctggcggcggcggctccggtggtggtggttcccaggtgcagctggtggagtctgggggaggcgtggtccagcctgggaggtccctgagactctcctgtgcagcgtctggattcatcttcagtcgctatggcatgcactgggtccgccaggctccaggcaaggggctgaaatgggtggcagttatatggtatgatggaagtaataaactctatgcagactccgtgaagggccgattcaccatctccagagacaattccaagaacacgctgtatctgcaaatgaacagcctgagagccgaggacacggctgtgtattactgtgcgagagattactatgataatagtagacatcactgggggtttgactactggggccagggaaccctggtcaccgtctcctcaggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaatag

SEQ ID NO: 111 - protein, 499 aa, Homo Sapiens
DIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKGGGGSGGGGSGGGGSQVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 112 - DNA, 1635 bp, Homo Sapiens
atggaaaccccagcgcagcttctcttcctcctgctactctggctcccagataccaccggagatattgtgatgacccagactccactctccctgcccgtcacccctggagagccggcctccatctcctgcaggtctagtcggagcctcttggatagtgatgatggaaacacctatttggactggtacctgcagaagccagggcagtctccacagctcctgatctacacgctttcctatcgggcctctggagtcccagacaggttcagtggcagtgggtcaggcactgatttcacactgaaaatcagcagggtggaggctgaggatgttggagtttattactgcatgcaacgtgtagagtttcctatcaccttcggccaagggacacgactggagattaaactttccgcggacgatgcgaaaaaggatgctgcgaagaaagatgacgctaagaaagacgatgctaaaaaggacctgcaggtgcagctggtggagtctgggggaggcgtggtccagcctgggaggtccctgagactctcctgtgcagcgtctggattcatcttcagtcgctatggcatgcactgggtccgccaggctccaggcaaggggctgaaatgggtggcagttatatggtatgatggaagtaataaactctatgcagactccgtgaagggccgattcaccatctccagagacaattccaagaacacgctgtatctgcaaatgaacagcctgagagccgaggacacggctgtgtattactgtgcgagagattactatgataatagtagacatcactgggggtttgactactggggccagggaaccctggtcaccgtctcctcaggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcactttccgcggacgatgcgaaaaaggatgctgcgaagaaagatgacgctaagaaagacgatgctaaaaaggacctggacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaagattataaggacgatgatgacaaatag

SEQ ID NO: 113 - protein, 524 aa, Homo Sapiens
DIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKLSADDAKKDAAKKDDAKKDDAKKDLQVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSLSADDAKKDAAKKDDAKKDDAKKDLDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKDYKDDDDK

SEQ ID NO: 114 - protein, 169 aa, Homo Sapiens
WVLSQVQLQESGPGLVKPSETLSLTCTVSGGSVSSGGYYWSWIRQPPGKGLEWIGFIYYTGSTNYNPSLKSRVSISVDTSKNQFSLKLSSVTAADAAVYYCARDYDWSFHFDYWGQGTLVTVSSASTKGPSVFPLAPCSRSTSESTAALGCLVKDYFPEPVTVSWNSGA

SEQ ID NO: 115 - protein, 168 aa, Homo Sapiens
QLLGLLLLWFPGARCDIQMTQSPSSLSASIGDRVTITCRASQGIRNDLGWYQQKPGKAPKRLIYAASSLQSGVPSRFSGSGSGTEFTLTISSLQPEDFATYYCLQHNSYPLTFGGGTKVEIKRTVAAPSVFIFPPSDEQLKSGTASVVCLLNNFYPREAKVQWKVDNA

SEQ ID NO: 116 - protein, 156 aa, Homo Sapiens
QCEVQLVESGGGLVQPGGSLRLSCAASGFTFTNYWMSWVRQAPGKGLEWVANIQQDGSEKYYVDSVRGRFTISRDNAKNSLYLQMNSLRAEDSAVYYCARWDYWGQGTLVTVSSASTKGPSVFPLAPCSRSTSESTAALGCLVKDYFPEPVSGVVE

SEQ ID NO: 117 - protein, 151 aa, Homo Sapiens
LLGLLMLWVPGSSGDIVMTQTPLSSTVILGQPASISCRSSQSLVHSDGNTYLNWLQQRPGQPPRLLIYMISNRFSGVPDRFSGSGAGTDFTLKISRVEAEDVGVYYCMQATESPQTFGQGTKVEIKRTVAAPSVFIFPPSDEQLKSGRASV

SEQ ID NO: 118 - protein, 180 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
XXXXEQSGGGVVKPGGSLRLSCAASGFTFSNAWMTWVRQAPGKGLEWVGRIKRRTDGGTTDYAAPVKGRFTISRDDSKNTLYLQMNNLKNEDTAVYYCTSVDNDVDYWGQGTLVTVSSASTKGPSVFPLAPCSRSTSESTAALGCLVKDYFPEPVTVSWNSGALTSGVHTFPAVLQSSGL

SEQ ID NO: 119 - protein, 152 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
XXXLTQSPLSLPVTPGEPASISCRSSQSLLHSNGYNYLDWYLQKPGQSPQLLIYLGSNRASGVPDRFSGSGSGTDFTLKISRVEAEDIGLYYCMQALQTPLTFGGGTKVDIKRTVAAPSVFIFPPSDEQLKSGTASVVCLLNNFYPREAKVQ

SEQ ID NO: 120 - protein, 179 aa, Homo Sapiens
QVQLEQSGGGLVQPGGSLRLSCAASGFTFSTYSMNWVRQAPGKGLEWVSYIRSSTSTIYYAESLKGRFTISSDNAKNSLYLQMNSLRDEDTAVYYCARDFDYWGQGTLVTVSSASTKGPSVFPLAPCSRSTSESTAALGCLVKDYFPEPVTVSWNSGALTSGVHTFPAVLQSSGLYSLS

SEQ ID NO: 121 - protein, 163 aa, Homo Sapiens
EIQLTQSPLSSPVTLGQPASISCRSSQSLVHSDGDTYLNWLQQRPGQPPRLLIYKISTRFSGVPDRFSGSGAGTDFTLKISRVETDDVGIYYCMQTTQIPQITFGQGTRLEIKRTVAAPSVFIFPPSDEQLKSGTASVVCLLNNFYPREAKVQWKVDNALQSG

SEQ ID NO: 122 - protein, 189 aa, Homo Sapiens
QVQLEQSGGGVVQPGRSLRLSCAASGFTFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSASTKGPSVFPLAPCSRSTSESTAALGCLVKDYFPEPVTVSWNSGALTSGVHTFPAVLQSSGLYSLS

SEQ ID NO: 123 - protein, 159 aa, Homo Sapiens
DIQLMTLQSPSSLSASVGDRVTITCRASQSIYSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSG

SEQ ID NO: 124 - protein, 181 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
  misc_feature (5)..(5): Wherein Xaa may be any amino acid
XXXXXQSGGGLVKPGGSLRLSCAASGFTFSNAWMTWVRQAPGKGLEWVGRIKRKTDGGTTDYAAPVKGRFTISRDDSENTLYLQMNSLETEDTAVYYCTTVDNSGDYWGQGTLVTVSSASTKGPSVFPLAPCSRSTSESTAALGCLVKDYFPEPVTVSWNSGALTSGVHTFPAVLQSSGLS

SEQ ID NO: 125 - protein, 159 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
XXXXTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNAL

SEQ ID NO: 126 - protein, 179 aa, Homo Sapiens
QVQLEQSGGGVVQPGRSLRLSCAASGFTFTNYGLHWVRQAPGKGLDWVAVIWYDGSHKFYADSVKGRFTISRDNSKNTLFLQMNSLRAEDTAVYYCTRDLDYWGQGTLVTVSSASTKGPSVFPLAPCSRSTSESTAALGCLVKDYFPEPVTVSWNSGALTSGVHTFPAVLQSSGLYSLS

SEQ ID NO: 127 - protein, 160 aa, Homo Sapiens
ETQLTQSPGTLSLSPGERVTLSCRASQSVSNNYLAWYQQKPGQAPRLLIYGASSRATGIPDRFSGSGSGTDFTLTISRLEPEDCAECYCQQYGSSLPLTFGGGTKVEIKRTVAAPSVFIFPPSDEQLKSGTASVVCLLNNFYPREAKVQWEGGITPSNRV

SEQ ID NO: 128 - protein, 182 aa, Homo Sapiens
VQCQVQLVESGGGVVQPGRSLRLSCAASGFTFSSYGMHWVRQAPGKGLEWVAVIWYDGSHKYLYATDSVKGRFTISRDNSKNTLYLQMNSLRAEDTAVYYSARDYYDTSRHHWGFDCWGQGTLVTVSSASTKGPSVFPLAPCSRSTSESTAALGCLVKDYFPEPVTVSWNSGALTSGVHTFP

SEQ ID NO: 129 - protein, 173 aa, Homo Sapiens
QLLGLLMLWVPGSSEEIVMTQTPLSLPVTPGEPASISCRSSQSLLDSEDGNTYLDWYLQKPGQSPQLLIYTLSHRASGVPDRFSGSGSGTDFTLKISRVEAEDVGVYCCMQRVEFPITFGQGTRLEIKRTVAAPSVFIFPPSDEQLKSGTASVVCLLNNFYPREAKVQWKVDN

SEQ ID NO: 130 - protein, 187 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
  misc_feature (5)..(5): Wherein Xaa may be any amino acid
XXXXXQSGPRLVKPSQTLSLTCTVSGGSISSDGYYWSWIRQHPGKGLEWIGYIYYSGSTFYNPSLKSRVAISVDTSKNQFSLKLSSVTAADTAVYYCARESPHSSNWYSGFDCWGQGTLVTVSSASTKGPSVFPLAPCSRSTSESTAALGCLVKDYFPRTGDGVVELRRPDQRRAHLPGCPTVLRTL

SEQ ID NO: 131 - protein, 154 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
XXXXTQSPDFQSVTPKEKVTITCRASQSIGSRLHWYQQKPDQSPKLLIKYASQSFSGVPSRFSGSGSGTDFTLTINSLEAEDAATYYCHQSSNLPFTFGPGTKVDIKRTVAAPSVFIFPPSDEQLKSGTASVVCLLNNFYPREAKVQWKVDNAL

SEQ ID NO: 132 - protein, 180 aa, Homo Sapiens
QVQLVEQAGGGVVQPGRSLRLSCAASGFTFRSYGMHWVRQAPGKGLKWVAVIWYDGSNKYLYTDSVKGRFTISRDNSKNTLYLQMNSLRAEDTAVYYCVRDYYDNSRHHWGFDYWGQGTLVTVSSASTKGPSVFPLAPCSRSTSESTAALGCLVKDYFPEPVTVSWNSGALTRRRAHLPG

SEQ ID NO: 133 - protein, 156 aa, Homo Sapiens
DIQMTQSPSSRCASVGDRVTITCRASQGIRNDLAWYQQKPGKAPKRLIYAASSLQSGVPSRFSGSRSGTEFTLTISSLQPEDFAAYYCLQHNSYPPSFGQGTKLEIKRTVAAPSVFIFPPSDEQLKSGTASVVCLLNNFYPREAKVQWKVDNALQS

SEQ ID NO: 134 - protein, 171 aa, Homo Sapiens
HVQVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSASTKGPSVFPLAPCSRSTSESTAALGCLVKDYFPEPVTVSWNSGAL

SEQ ID NO: 135 - protein, 174 aa, Homo Sapiens
SAPGAANALGPWISEDIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKRTVAAPSVFIFPPSDEQLKSGTASVVCLLNNFYPREAKVQWKVDNA

SEQ ID NO: 136 - DNA, 1428 bp, Homo Sapiens
cggccgcctatttacccagagacagggagaggctcttctgtgtgtagtggttgtgcagagcctcatgcatcacggagcatgagaagacattcccctcctgccacctgctcttgtccacggttagcctgctgtagaggaagaaggagccgtcggagtccagcacgggaggcgtggtcttgtagttgttctccggctgcccattgctctcccactccacggcgatgtcgctggggtagaagcctttgaccaggcaggtcaggctgacctggttcttggtcatctcctcctgggatgggggcagggtgtacacctgtggctctcggggctgccctttggctttggagatggttttctcgatggaggacgggaggcctttgttggagaccttgcacttgtactccttgccgttcagccagtcctggtgcaggacggtgaggacgctgaccacacggtacgtgctgttgaactgctcctcccgcggctttgtcttggcattatgcacctccacgccatccacgtaccagttgaactggacctcggggtcttcctggctcacgtccaccaccacgcacgtgacctcaggggtccgggagatcatgagagtgtccttgggttttggggggaacaggaagactgatggtccccccaggaactcaggtgctgggcatgatgggcatgggggaccatatttggactcaactctcttgtccaccttggtgttgctgggcttgtgatctacgttgcaggtgtaggtcttcgtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggcggctgtgctctcggaggtgctcctggagcagggcgccagggggaagacggatgggcccttggtggaagctgaggagacggtgaccagggttccctggccccagtagtcaaacccccagtgatgtctactattatcatagtaatctctcgcacagtaatacacagccgtgtcctcggctctcaggctgttcatttgcagatacagcgtgttcttggaattgtctctggagatggtgaatcggcccttcacggagtctgcatagagtttattacttccatcataccatataactgccacccatttcagccccttgcctggagcctggcggacccagtgcatgccatagcgactgaagatgaatccagacgctgcacaggagagtctcagggacctcccaggctggaccacgcctcccccagactccaccagctgcacctgacactggacaccttttaaaatagccacaagaaaaagccagctcagcccaaactccatggtggtcgact

SEQ ID NO: 137 - protein, 469 aa, Homo Sapiens
MEFGLSWLFLVAILKGVQCQVQLVESGGGVVQPGRSLRLSCAASGFIFSRYGMHWVRQAPGKGLKWVAVIWYDGSNKLYADSVKGRFTISRDNSKNTLYLQMNSLRAEDTAVYYCARDYYDNSRHHWGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - DNA, 741 bp, Homo Sapiens
agtcgaccaccatggaaaccccagcgcagcttctcttcctcctgctactctggctcccagataccaccggagatattgtgatgacccagactccactctccctgcccgtcacccctggagagccggcctccatctcctgcaggtctagtcggagcctcttggatagtgatgatggaaacacctatttggactggtacctgcagaagccagggcagtctccacagctcctgatctacacgctttcctatcgggcctctggagtcccagacaggttcagtggcagtgggtcaggcactgatttcacactgaaaatcagcagggtggaggctgaggatgttggagtttattactgcatgcaacgtgtagagtttcctatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggcggccg

SEQ ID NO: 139 - protein, 240 aa, Homo Sapiens
METPAQLLFLLLLWLPDTTGDIVMTQTPLSLPVTPGEPASISCRSSRSLLDSDDGNTYLDWYLQKPGQSPQLLIYTLSYRASGVPDRFSGSGSGTDFTLKISRVEAEDVGVYYCMQRVEFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 186 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
XXXXEQSGGGVVQPGRSLRLSCAASGFTFSSYGMYWVRQAPGKGLEWVAVIWYDGSNKYYADSVKGRFTISRDNSKNTLYLQMNSLRAEDTAVYYCARDFYDSSRYHYGMDVWGQGTTVTVSSASTKGPSVFPLAPCSRSTSESTAALGCLVKDYFPEPVTVSWNSGALTSGVHTFPAVLQSSGLS

SEQ ID NO: 141 - protein, 143 aa, Homo Sapiens
  misc_feature (1)..(1): Wherein Xaa may be any amino acid
  misc_feature (2)..(2): Wherein Xaa may be any amino acid
  misc_feature (3)..(3): Wherein Xaa may be any amino acid
  misc_feature (4)..(4): Wherein Xaa may be any amino acid
XXXXTQCPLSLPVTPGEPASISCRSSQSLLDSDDGNTYLDWYLQKPGQSPQLLIYTVSYRASGVPDRFSGSGSGTDFTLKISRVEAEDVGVYYCMQRIEFPITFGQGTRLEIKRTVAAPSVFIFPPSDEQLKSGTASVVCLLN

(19) United States
(12) Patent Application Publication
(10) Pub. No.: US 2017/0267750 A1
(43) Pub. Date: Sep. 21, 2017

(54) Method of Treating Ovarian and Renal Cancer Using Antibodies Against T Cell Immunoglobulin Domain and Mucin Domain 1 (TIM-1) Antigen

(71) Applicant: Celldex Therapeutics, Inc., Hampton, NJ (US)

(72) Inventors: Kam Fai Tse, Clinton, CT (US); Vincent A. Pollack, Gales Ferry, CT (US); John MacDougall, Hamden, CT (US); Suresh G. Shenoy, Branford, CT (US); Traci Mansfield, Guilford, CT (US); Henri Lichenstein, Guilford, CT (US); Michael E. Jeffers, Branford, CT (US); William J. LaRochelle, Madison, CT (US)

(21) Appl. No.: 15/465,994

(22) Filed: Mar. 22, 2017

Related U.S. Application Data
(63) Continuation of application No. 13/862,510, filed on Apr. 15, 2013, now abandoned.
Application No. 13/862,510 is a continuation of application No. 13/591,799, filed on Aug. 22, 2012, now abandoned.
Application No. 13/591,799 is a continuation of application No. 13/346,129, filed on Jan. 9, 2012, now abandoned.
Application No. 13/346,129 is a continuation of application No. 13/113,692, filed on May 23, 2011, now abandoned.
Application No. 13/113,692 is a continuation of application No. 12/897,012, filed on Oct. 4, 2010, now abandoned.
Application No. 12/897,012 is a continuation of application No. 12/707,146, filed on Feb. 17, 2010, now abandoned.
Application No. 12/707,146 is a continuation of application No. 12/084,914, now abandoned.
(60) Provisional application No. 60/735,574, filed on Nov. 10, 2005.

(51) Int. Cl. C07K 16/18; A61K 51/10; B82Y 5/00; C07K 16/28; C07K 16/30; C07K 16/42; G01N 33/574; A61K 39/00

(52) U.S. Cl. CPC C07K 16/18; A61K 51/106; A61K 47/48469; A61K 47/48561; A61K 47/48638; B82Y 5/00; C07K 16/2803; C07K 16/2809; C07K 16/3038; C07K 16/3069; G01N 33/57438; G01N 33/57449; C07K 16/42; A61K 47/48607; A61K 47/48746; A61K 51/1027; A61K 51/1072; A61K 2039/505; C07K 2317/21; C07K 2317/31; C07K 2317/73; C07K 2317/92; G01N 2333/705; C07K 2317/34; C07K 2317/622; C07K 2317/76

(57) ABSTRACT

The invention described herein is related to antibodies directed to the antigen TIM-1 and uses of such antibodies for the treatment of cancer (e.g., renal and ovarian cancer). In particular, there are provided fully human monoclonal antibodies directed to the antigen TIM-1. Isolated polynucleotide sequences encoding, and amino acid sequences comprising, heavy and light chain immunoglobulin molecules, particularly sequences corresponding to contiguous heavy and light chain sequences spanning the framework regions (FR's) and/or complementarity determining regions (CDR's), specifically from FR1 through FR4 or CDR1 through CDR3, are provided. Hybridomas or other cell lines expressing such immunoglobulin molecules and monoclonal antibodies are also provided.

### RELATED APPLICATIONS

This application is a continuation of U.S. patent application Ser. No. 13/862,510, filed Apr. 15, 2013, which is a continuation of U.S. patent application Ser. No. 13/591,799, filed Aug. 22, 2012, which is a continuation of U.S. patent application Ser. No. 13/346,129, filed Jan. 9, 2012, which is a continuation of U.S. patent application Ser. No. 13/113,692, filed May 23, 2011, which is a continuation of U.S. patent application Ser. No. 12/897,012, filed Oct. 4, 2010, which is a continuation of U.S. patent application Ser. No. 12/707,146, filed Feb. 17, 2010, which is a continuation of U.S. patent application Ser. No. 12/084,914, which was deposited on May 12, 2008 as a national stage application, filed under 35 U.S.C. §371, of International Application No. PCT/US2006/044090, filed on Nov. 13, 2006 which claims the benefit of U.S. Ser. No. 60/735,574, filed Nov. 10, 2005, each of which is herein incorporated by reference in its entirety.

### INCORPORATION-BY-REFERENCE OF SEQUENCE LISTING

The contents of the text file named “965AUSseqlist.txt,” which was created on Oct. 4, 2010 and is 131 KB in size, are hereby incorporated by reference in their entirety.

### BACKGROUND OF THE INVENTION

Field of the Invention

The invention disclosed herein is related to antibodies directed to the antigen T cell, immunoglobulin domain and mucin domain 1 (TIM-1) proteins and uses of such antibodies. In particular, there are provided fully human monoclonal antibodies directed to the antigen TIM-1. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDRs), specifically from FR1 through FR4 or CDR1 through CDR3, are provided. Hybridomas or other cell lines expressing such immunoglobulin molecules and monoclonal antibodies are also provided.

Description of the Related Art

A new family of genes encoding T cell, immunoglobulin domain and mucin domain (TIM) proteins (three in humans and eight in mice) have been described recently with emerging roles in immunity. Kuchroo et al., Nat Rev Immunol 3:454-462 (2003); McIntire et al., Nat Immunol 2:1109-1116 (2001). The TIM gene family members reside in chromosomal regions, 5q33.2 in human and 11B1.1 in mouse, and have been linked to allergy and autoimmune diseases. Shevach, Nat Rev Immunol 2:389-400 (2002); Wills-Karp et al., Nat Immunol 4:1050-1052 (2003).

One TIM family member, TIM-1, is also known as Hepatitis A virus cellular receptor (HAVcr-1) and was originally discovered as a receptor for Hepatitis A virus (HAV) (Kaplan et al, EMBO J 15(16):4282-96 (1996)). This gene was later cloned as kidney injury molecule 1 (KIM-1) (Ichimura et al., J Biol Chem 273:4135-4142 (1998); Han et al., Kidney Int 62:237-244 (2002)).

Kaplan et al. isolated the cellular receptor for hepatitis A virus from a cDNA library from a primary African Green Monkey Kidney (AGMK) cell line expressing the receptor. See U.S. Pat. No. 5,622,861. The disclosed utility of the polypeptides and nucleic acids was to diagnose infection by hepatitis A virus, to separate hepatitis A virus from impurities in a sample, to treat infection as well as to prevent infection by hepatitis A virus. Furthermore, the polypeptides could be expressed in transformed cells and used to test efficacy of compounds in an anti-hepatitis A virus binding assay.

The human homolog, hHAVcr-1 (aka TIM-1), was described by Feigelstock et al., J Virology 72(8): 6621-6628 (1998). The same molecules were described in PCT Publication Nos: WO 97/44460 and WO 98/53071 and U.S. Pat. No. 6,664,385 as Kidney Injury-related Molecules (KIM) that were found to be upregulated in renal tissue after injury to the kidney. The molecules were described as being useful in a variety of therapeutic interventions, specifically, renal disease, disorder or injury. For example, PCT Publication No. WO 02/098920 describes antibodies to KIM and describes antibodies that inhibit the shedding of KIM-1 polypeptide from KIM-1 expressing cells e.g., renal cells, or renal cancer cells.

TIM-1 is a type 1 membrane protein that contains a novel six-cysteine immunoglobulin-like domain and a mucin threonine/serine.proline-rich (T/S/P) domain. TIM-1 was originally identified in rat. TIM-1 has been found in mouse, African green monkey, and humans (Feigelstock et al., J Virol 72(8):6621-8 (1998). The African green monkey ortholog is most closely related to human TIM-1 showing 77.6% amino acid identity over 358 aligned amino acids. Rat and mouse orthologs exhibit 50% (155/310) and 45.6% (126/276) amino acid identity respectively, although over shorter segments of aligned sequence than for African green monkey. Monoclonal antibodies to the Ig-like domain of TIM-1 have been shown to be protective against Hepatitis A Virus infection in vitro. Silberstein et al., J Virol 75(2):717-25 (2001). In addition, Kim-1 was shown to be expressed at low levels in normal kidney but its expression is increased dramatically in postischemic kidney. Ichimura et al., J Biol Chem 273(7):4135-42 (1998). HAVCR-1 is also expressed at elevated levels in clear cell carcinomas and cancer cell lines derived from the same.

TIM-1 shows homology to the P-type “trefoil” domain suggesting that it may have similar biological activity to other P-type trefoil family members. Some trefoil domain containing proteins have been shown to induce cellular scattering and invasion when used to treat kidney, colon and breast tumor cell lines. Prest et al., FASEB J 16(6):592-4 (2002). In addition, some trefoil containing proteins confer cellular resistance to anoikis, an anchorage-related apoptosis phenomenon in epithelium. Chen et al., Biochem Biophys Res Commun 274(3):576-82 (2000).

TIM-1 maps to a region of human chromosome 5 known as Tapr in the murine sytenic region that has been implicated in asthma. Tapr, a major T cell regulatory locus, controls the development of airway hyperreactivity. Wills-Karp, Nature Immunology 2:1095-1096 (2001); McIntire et al., Nature Immunology 2:1109-1116 (2001).

### SUMMARY OF THE INVENTION

Embodiments of the invention described herein are based upon the development of human monoclonal antibodies, or binding fragments thereof, that bind TIM-1 and affect TIM-1 function. TIM-1 is expressed at elevated levels in pathologies, such as neoplasms and inflammatory diseases. Inhibition of the biological activity of TIM-1 can thus prevent inflammation and other desired effects, including TIM-1 induced cell proliferation. Embodiments of the invention are based upon the generation and identification of isolated antibodies, or binding fragments thereof, that bind specifically to TIM-1.

Accordingly, one embodiment of the invention includes isolated antibodies, or fragments of those antibodies, that specifically bind to TIM-1. As known in the art, the antibodies can advantageously be, for example, monoclonal, chimeric and/or fully human antibodies. Embodiments of the invention described herein also provide cells for producing these antibodies.

Some embodiments of the invention described herein relate to monoclonal antibodies that bind TIM-1 and affect TIM-1 function. Other embodiments relate to fully human anti-TIM-1 antibodies and anti-TIM-1 antibody preparations with desirable properties from a therapeutic perspective, including strong binding affinity for TIM-1, the ability to neutralize TIM-1 in vitro and in vivo, and the ability to inhibit TIM-1 induced cell proliferation.

In a preferred embodiment, antibodies described herein bind to TIM-1 with very high affinities (Kd). For example a human, rabbit, mouse, chimeric or humanized antibody that is capable of binding TIM-1 with a Kd less than, but not limited to, 10⁻⁷, 10⁻⁸, 10⁻⁸, 10⁻¹⁰, 10⁻¹¹, 10⁻¹², 10⁻¹³ or 10⁻¹⁴ M, or any range or value therein. Affinity and/or avidity measurements can be measured by KinExA® and/or BIACORE®, as described herein.

In one embodiment, the invention provides an isolated antibody that specifically binds to T cell, immunoglobulin domain and mucin domain 1 (TIM-1). In some embodiments, the isolated antibody has a heavy chain polypeptide comprising an amino acid sequence selected from the group consisting of SEQ ID NOs: 2, 6, 10, 14, 18, 22, 26, 30, 34, 38, 42, 46, and 50.

In another embodiment, the invention provides an isolated antibody that specifically binds to T cell, immunoglobulin domain and mucin domain 1 (TIM-1) and has a light chain polypeptide comprising an amino acid sequence selected from the group consisting of SEQ ID NOs: 4, 8, 12, 16, 20, 24, 28, 32, 36, 40, 44, 48, and 52.

In yet another embodiment, the invention provides an isolated antibody that specifically binds to TIM-1 and has a heavy chain polypeptide comprising an amino acid sequence selected from the group consisting of SEQ ID NOs: 2, 6, 10, 14, 18, 22, 26, 30, 34, 38, 42, 46, and 50 and has a light chain polypeptide comprising an amino acid sequence selected from the group consisting of SEQ ID NOs: 4, 8, 12, 16, 20, 24, 28, 32, 36, 40, 44, 48, and 52.

Another embodiment of the invention is a fully human antibody that specifically binds to TIM-1 and has a heavy chain polypeptide comprising an amino acid sequence comprising the complementarity determining region (CDR) with one of the sequences shown in Table 4. It is noted that CDR determinations can be readily accomplished by those of ordinary skill in the art. See for example, Kabat et al., Sequences of Proteins of Immunological Interest, Fifth Edition, NIH Publication 91-3242, Bethesda Md. [1991], vols. 1-3.

Yet another embodiment is an antibody that specifically binds to TIM-1 and has a light chain polypeptide comprising an amino acid sequence comprising a CDR comprising one of the sequences shown in Table 5. In certain embodiments the antibody is a fully human monoclonal antibody.

A further embodiment is an antibody that binds to TIM-1 and comprises a heavy chain polypeptide comprising an amino acid sequence comprising one of the CDR sequences shown in Table 4 and a light chain polypeptide comprising an amino acid sequence comprising one of the CDR sequences shown in Table 5. In certain embodiments the antibody is a fully human monoclonal antibody.

Another embodiment of the invention is a fully human antibody that binds to orthologs of TIM-1. A further embodiment herein is an antibody that cross-competes for binding to TIM-1 with the fully human antibodies described herein.

Other embodiments includes methods of producing high affinity antibodies to TIM-1 by immunizing a mammal with human TIM-1, or a fragment thereof, and one or more orthologous sequences or fragments thereof.

It will be appreciated that embodiments of the invention are not limited to any particular form of an antibody. For example, the anti-TIM-1 antibody can be a full length antibody (e.g., having an intact human Fc region) or an antibody fragment (e.g., a Fab, Fab′, F(ab′)₂, Fv, or single chain antibodies). In addition, the antibody can be manufactured from a hybridoma that secretes the antibody, or from a recombinantly produced cell that has been transformed or transfected with a gene or genes encoding the antibody.

Some embodiments of the invention include isolated nucleic acid molecules encoding any of the anti-TIM-1 antibodies described herein, vectors having an isolated nucleic acid molecule encoding the anti-TIM-1 antibody, and a host cell transformed with such a nucleic acid molecule. In addition, one embodiment of the invention is a method of producing an anti-TIM-1 antibody by culturing host cells under conditions wherein a nucleic acid molecule is expressed to produce the antibody followed by recovering the antibody from the host cell.

In other embodiments the invention provides compositions, including an antibody, or functional fragment thereof, and a pharmaceutically acceptable carrier.

In some embodiments, the invention includes pharmaceutical compositions having an effective amount of an anti-TIM-1 antibody in admixture with a pharmaceutically acceptable carrier or diluent. In yet other embodiments, the anti-TIM-1 antibody, or a fragment thereof, is conjugated to a therapeutic agent. The therapeutic agent can be, for example, a toxin, a radioisotope, or a chemotherapeutic agent. Preferably, such antibodies can be used for the treatment of pathologies, including for example, tumors and cancers, such as ovarian, stomach, endometrial, salivary gland, lung, kidney, colon, colorectal, thyroid, pancreatic, prostate and bladder cancer, as well as other inflammatory conditions. More preferably, the antibodies can be used to treat renal and ovarian carcinomas.

In still further embodiments, the antibodies described herein can be used for the preparation of a medicament for the effective treatment of TIM-1 induced cell proliferation in an animal, wherein said monoclonal antibody specifically binds to TIM-1.

Yet another embodiment is the use of an anti-TIM-1 antibody in the preparation of a medicament for the treatment of diseases such as neoplasms and inflammatory conditions. In one embodiment, the neoplasm includes, without limitation, tumors and cancers, such as ovarian, stomach, endometrial, salivary gland, lung, kidney, colon, colorectal, thyroid, pancreatic, prostate and bladder cancer.

In yet another aspect, the invention includes a method for effectively treating pathologies associated with the expression of TIM-1. These methods include selecting an animal in need of treatment for a condition associated with the expression of TIM-1, and administering to said animal a therapeutically effective dose of a fully human monoclonal antibody, wherein said antibody specifically binds to TIM-1.

Preferably a mammal and, more preferably, a human, receives the anti-TIM-1 antibody. In a preferred embodiment, neoplasms are treated, including, without limitation, renal and pancreatic tumors, head and neck cancer, ovarian cancer, gastric (stomach) cancer, melanoma, lymphoma, prostate cancer, liver cancer, lung cancer, renal cancer, bladder cancer, colon cancer, esophageal cancer, and brain cancer.

Further embodiments of the invention include the use of an antibody of in the preparation of medicament for the effective treatment of neoplastic disease in an animal, wherein said monoclonal antibody specifically binds to TIM-1. Treatable neoplastic diseases include, for example, ovarian cancer, bladder cancer, lung cancer, glioblastoma, stomach cancer, endometrial cancer, kidney cancer, colon cancer, pancreatic cancer, and prostrate cancer.

In some embodiments, the invention includes a method for inhibiting cell proliferation associated with the expression of TIM-1. These methods include selecting an animal in need of treatment for TIM-1 induced cell proliferation and administering to said animal a therapeutically effective dose of a fully human monoclonal antibody, wherein the antibody specifically binds TIM-1. In other embodiments, cells expressing TIM-1 are treated with an effective amount of an anti-TIM-1 antibody or a fragment thereof. The method can be performed in vivo.

The methods can be performed in vivo and the patient is preferably a human patient. In a preferred embodiment, the methods concern the treatment of neoplastic diseases, for example, tumors and cancers, such as renal (kidney) cancer, pancreatic cancer, head and neck cancer, ovarian cancer, gastric (stomach) cancer, melanoma, lymphoma, prostate cancer, liver cancer, breast cancer, lung cancer, bladder cancer, colon cancer, esophageal cancer, and brain cancer.

In some embodiments, the anti-TIM-1 antibody is administered to a patient, followed by administration of a clearing agent to remove excess circulating antibody from the blood.

In some embodiments, anti-TIM-1 antibodies can be modified to enhance their capability of fixing complement and participating in complement-dependent cytotoxicity (CDC). In one embodiment, anti-TIM-1 antibodies can be modified, such as by an amino acid substitution, to alter their clearance from the body. Alternatively, some other amino acid substitutions can slow clearance of the antibody from the body.

In another embodiment, the invention provides an article of manufacture including a container. The container includes a composition containing an anti-TIM-1 antibody, and a package insert or label indicating that the composition can be used to treat neoplastic or inflammatory diseases characterized by the overexpression of TIM-1.

Yet another embodiment provides methods for assaying the level of TIM-1 in a patient sample, comprising contacting an anti-TIM-1 antibody with a biological sample from a patient, and detecting the level of binding between said antibody and TIM-1 in said sample. In more specific embodiments, the biological sample is blood.

In one embodiment, the invention includes an assay kit for detecting TIM-1 and TIM-1 orthologs in mammalian tissues or cells to screen for neoplastic diseases or inflammatory conditions. The kit includes an antibody that binds to TIM-1 and a means for indicating the reaction of the antibody with TIM-1, if present. Preferably the antibody is a monoclonal antibody. In one embodiment, the antibody that binds TIM-1 is labeled. In another embodiment the antibody is an unlabeled first antibody and the kit further includes a means for detecting the first antibody. In one embodiment, the means includes a labeled second antibody that is an anti-immunoglobulin. Preferably the antibody is labeled with a marker selected from the group consisting of a fluorochrome, an enzyme, a radionuclide and a radiopaque material.

Another embodiment of the invention includes a method of diagnosing diseases or conditions in which an antibody prepared as described herein is utilized to detect the level of TIM-1 in a patient sample. In one embodiment, the patient sample is blood or blood serum. In further embodiments, methods for the identification of risk factors, diagnosis of disease, and staging of disease is presented which involves the identification of the overexpression of TIM-1 using anti-TIM-1 antibodies.

Embodiments of the invention described herein also pertain to variants of a TIM-1 protein that function as either TIM-1 agonists (mimetics) or as TIM-1 antagonists.

Another embodiment of the invention is the use of monoclonal antibodies directed against the TIM-1 antigen coupled to cytotoxic chemotherapic agents or radiotherapic agents such as anti-tumor therapeutics.

One embodiment provides an isolated antibody that blocks simultaneous binding to TIM-1 antigen by an antibody having a heavy chain sequence comprising an the amino acid sequence selected from the group consisting of SEQ ID NOS: 2, 6, 10, 14, 18, 22, 26, 30, 34, 38, 42, 46, and 50. Another embodiment provides an isolated antibody that binds to TIM-1 antigen and that cross reacts with an antibody having a heavy chain sequence comprising the amino acid sequence from the group consisting of SEQ ID NOS: 2, 6, 10, 14, 18, 22, 26, 30, 34, 38, 42, 46, and 50.

Another embodiment of the invention provides an isolated antibody that binds to an epitope of SEQ ID NO: 87 on the TIM-1 antigen of SEQ ID NO: 50, and that cross reacts with an antibody having a heavy chain sequence comprising the amino acid sequence selected from the group consisting of SEQ ID NOS: 2, 6, 10, 14, 18, 22, 26, 30, 34, 38, 42, 46, and 54. In still another embodiment, the invention provides an isolated antibody that binds to an epitope of SEQ ID NO: 87 on the TIM-1 antigen of SEQ ID NO: 50, wherein said antibody blocks simultaneous binding to TIM-1 antigen by an antibody having a heavy chain sequence comprising the amino acid sequence selected from the group comprising SEQ ID NOS: 2, 6, 10, 14, 18, 22, 26, 30, 34, 38, 42, 46, and 54.

### BRIEF DESCRIPTION OF THE DRAWINGS

FIG. 1 is a bar graph of the results of an ELISA assay of anti-TIM-1 monoclonal antibodies 1.29, 2.56.2, 2.59.2, and 2.45.1 against the TIM-1 antigen.

FIG. 2 is a bar graph of the results of an ELISA assay of anti-TIM-1 monoclonal antibodies 1.29, 2.56.2, 2.59.2, and 2.45.1 against irrelevant protein.

FIGS. 3A-3B show staining of Renal Cell Cancer (FIG. 3A) and Pancreatic Cancer (FIG. 3B) with the anti-TIM-1 mAb 2.59.2.

FIG. 4 is a bar graph of clonogenic assay results of anti-TIM-1 monoclonal antibody mediated toxin killing in the ACHN kidney cancer cell line.

FIG. 5 is a bar graph of clonogenic assay results of anti-TIM-1 monoclonal antibody mediated toxin killing in the BT549 breast cancer cell line.

FIG. 6 is a bar graph of the results of a clonogenic assay of CAKI-1 cells treated with Auristatin E (AE) conjugated antibodies.

FIG. 7 is a bar graph of the results of a clonogenic assay of BT549 cells treated with Auristatin E (AE) conjugated antibodies.

FIG. 8 is a bar graph showing that anti-TIM-1 monoclonal antibodies 2.59.2, 2.56.2 and 2.45.1 significantly inhibit IL-4 release from Th1 cells compared to the control PK16.3 mAb.

FIG. 9 is a bar graph showing that anti-TIM-1 monoclonal antibodies 2.59.2 and 2.45.1 significantly inhibit IL-4 release from Th2 cells compared to control PK16.3 mAb.

FIG. 10 is a bar graph showing that anti-TIM-1 monoclonal antibody 2.59.2 significantly inhibited IL-5 release from Th1 cells compared to control PK16.3 mAb.

FIG. 11 is a bar graph showing that anti-TIM-1 monoclonal antibodies 2.59.2 and 1.29 significantly inhibited IL-5 release from Th2 cells compared to control PK16.3 mAb.

FIG. 12 is a bar graph showing that anti-TIM-1 monoclonal antibodies 2.59.2, 1.29 and 2.56.2 significantly inhibited IL-10 release from Th1 cells compared to control PK16.3 mAb.

FIG. 13 is a bar graph showing that anti-TIM-1 monoclonal antibodies 2.59.2, 1.29 and 2.45.1 significantly inhibited IL-10 release from Th2 cells compared to control PK16.3 mAb.

FIG. 14 is a bar graph showing that anti-TIM-1 monoclonal antibodies 2.59.2, 1.29 and 2.56.2 significantly inhibited IL-13 release from Th1 cells compared to control PK16.3 mAb.

FIG. 15 is a bar graph showing that anti-TIM-1 monoclonal antibodies 2.59.2 and 1.29 significantly inhibited IL-13 release from Th2 cells compared to control PK16.3 mAb.

FIG. 16 is a bar graph showing that anti-TIM-1 monoclonal antibodies did not inhibit IFNγ release from Th1 cells compared to control PK16.3 mAb.

FIG. 17 is a bar graph showing that anti-TIM-1 monoclonal antibodies 2.59.2 and 2.45.1 significantly inhibited IFNγ release from Th2 cells compared to control PK16.3 mAb.

FIGS. 18A-18T are bar graphs showing BrdU incorporation assay results from experiments in which the neutralization of various human anti-TIM-1 monoclonal antibodies was assessed.

FIGS. 19A through 19D are line graphs showing the results of antibody conjugate studies performed using the plant toxin Saporin conjugated to TIM-1-specific antibodies and irrelevant antibodies (FIGS. 19A-19C). Additional negative controls included irrelevant antibodies alone without toxin (FIG. 19D).

FIG. 20 is a graph showing tumor growth inhibition and complete regression of IGROV1 ovarian carcinoma xenografts in athymic mice after treatment with 6.25 to 50 mg/kg i.v. every 4 days for 4 treatments. The responses of tumor-bearing animals to reference drugs such as vinblastine (1.7 mg/kg i.v. q4d×4) and paclitaxel (15.0 mg/kg i.v. q2d×4) are also shown. Control groups were treated with either phosphate-buffered saline (PBS) or physiological saline. CR014-vcMMAE was toxic to the test animals at 50 mg/kg/treatment (n=1/6) and at 100 mg/kg/treatment (n=6/6).

### DETAILED DESCRIPTION OF THE PREFERRED EMBODIMENT

Embodiments of the invention described herein are based upon the generation and identification of isolated antibodies that bind specifically to T cell, immunoglobulin domain and mucin domain 1 (TIM-1). As discussed below, TIM-1 is expressed at elevated levels in clear cell carcinomas and cancer cell lines derived from the same. Accordingly, antibodies that bind to TIM-1 are useful for the treatment and inhibition of carcinomas. In addition, antibodies that bind TIM-1 are also useful for reducing cell migration and enhancing apoptosis of kidney cancer cells.

Accordingly, embodiments of the invention described herein provide isolated antibodies, or fragments of those antibodies, that bind to TIM-1. As known in the art, the antibodies can advantageously be, e.g., monoclonal, chimeric and/or human antibodies. Embodiments of the invention described herein also provide cells for producing these antibodies.

Another embodiment of the invention provides for using these antibodies for diagnostic or therapeutic purposes. For example, embodiments of the invention provide methods and antibodies for inhibiting the expression of TIM-1 associated with cell proliferation. Preferably, the antibodies are used to treat neoplasms such as renal and pancreatic tumors, head and neck cancer, ovarian cancer, gastric (stomach) cancer, melanoma, lymphoma, prostate cancer, liver cancer, breast cancer, lung cancer, renal cancer, bladder cancer, colon cancer, esophageal cancer, and brain cancer. In association with such treatment, articles of manufacture comprising these antibodies are provided. Additionally, an assay kit comprising these antibodies is provided to screen for cancers or tumors.

Additionally, the nucleic acids described herein, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully in the following disclosure.

Furthermore, the TIM-1 proteins and polypeptides described herein, and fragments and variants thereof, may be used, in ways that include (a) serving as an immunogen to stimulate the production of an anti-TIM-1 antibody, (b) a capture antigen in an immunogenic assay for such an antibody, (c) as a target for screening for substances that bind to a TIM-1 polypeptide described herein, and (d) a target for a TIM-1 specific antibody such that treatment with the antibody affects the molecular and/or cellular function mediated by the target. TIM-1 polypeptide expression or activity can promote cell survival and/or metastatic potential. Conversely, a decrease in TIM-1 polypeptide expression or inhibition of its function reduces tumor cell survival and invasiveness in a therapeutically beneficial manner.

Single chain antibodies (scFv's) and bispecific antibodies specific for TIM-1 are useful particularly because it may more readily penetrate a tumor mass due to its smaller size relative to a whole IgG molecule. Studies comparing the tumor penetration between whole IgG molecules and scFv's have been have been described in the literature. The scFv can be derivatized with a toxin or radionuclide in order to destroy tumor cells expressing the TIM-1 antigen, in a manner similar to the IgG2 or IgG4 anti-TIM-1 toxin labeled or radionuclide derivatized whole antibodies already discussed, but with the advantage of being able to penetrate the tumor more fully, which may translate into increased efficacy in eradicating the tumor. A specific example of a biologically active anti-TIM-1 scFv is provided herein.

### SEQUENCE LISTING

The heavy chain and light chain variable region nucleotide and amino acid sequences of representative human anti-TIM-1 antibodies are provided in the sequence listing, the contents of which are summarized in Table 1 below.

TABLE 1

| mAb | | SEQ |
|---|---|---|
| ID No.: | Sequence | ID NO: |
| 1.29 | Nucleotide sequence encoding the variable region and a portion of the | 1 |
| | constant region of the heavy chain | |
| | Amino acid sequence of the variable region of the heavy chain | 2 |
| | Nucleotide sequence encoding the variable region and a portion of the | 3 |
| | constant region of the light chain | |
| | Amino acid sequence of the variable region of the light chain | 4 |
| 1.37 | Nucleotide sequence encoding the variable region and a portion of the | 5 |
| | constant region of the heavy chain | |
| | Amino acid sequence of the variable region of the heavy chain | 6 |
| | Nucleotide sequence encoding the variable region and a portion of the | 7 |
| | constant region of the light chain | |
| | Amino acid sequence of the variable region of the light chain | 8 |
| 2.16 | Nucleotide sequence encoding the variable region and a portion of the | 9 |
| | constant region of the heavy chain | |
| | Amino acid sequence of the variable region of the heavy chain | 10 |
| | Nucleotide sequence encoding the variable region and a portion of the | 11 |
| | constant region of the light chain | |
| | Amino acid sequence of the variable region of the light chain | 12 |
| 2.17 | Nucleotide sequence encoding the variable region and a portion of the | 13 |
| | constant region of the heavy chain | |
| | Amino acid sequence of the variable region of the heavy chain | 14 |
| | Nucleotide sequence encoding the variable region and a portion of the | 15 |
| | constant region of the light chain | |
| | Amino acid sequence of the variable region of the light chain | 16 |
| 2.24 | Nucleotide sequence encoding the variable region and a portion of the | 17 |
| | constant region of the heavy chain | |
| | Amino acid sequence of the variable region of the heavy chain | 18 |
| | Nucleotide sequence encoding the variable region and a portion of the | 19 |
| | constant region of the light chain | |
| | Amino acid sequence of the variable region of the light chain | 20 |
| 2.45 | Nucleotide sequence encoding the variable region and a portion of the | 21 |
| | constant region of the heavy chain | |
| | Amino acid sequence of the variable region of the heavy chain | 22 |
| | Nucleotide sequence encoding the variable region and a portion of the | 23 |
| | constant region of the light chain | |
| | Amino acid sequence of the variable region of the light chain | 24 |
| 2.54 | Nucleotide sequence encoding the variable region and a portion of the | 25 |
| | constant region of the heavy chain | |
| | Amino acid sequence of the variable region of the heavy chain | 26 |
| | Nucleotide sequence encoding the variable region and a portion of the | 27 |
| | constant region of the light chain | |
| | Amino acid sequence of the variable region of the light chain | 28 |
| 2.56 | Nucleotide sequence encoding the variable region and a portion of the | 29 |
| | constant region of the heavy chain | |
| | Amino acid sequence of the variable region of the heavy chain | 30 |
| | Nucleotide sequence encoding the variable region and a portion of the | 31 |
| | constant region of the light chain | |
| | Amino acid sequence of the variable region of the light chain | 32 |
| 2.59 | Nucleotide sequence encoding the variable region and a portion of the | 33 |
| | constant region of the heavy chain | |
| | Amino acid sequence of the variable region of the heavy chain | 34 |
| | Nucleotide sequence encoding the variable region and a portion of the | 35 |
| | constant region of the light chain | |
| | Amino acid sequence of the variable region of the light chain | 36 |
| 2.61 | Nucleotide sequence encoding the variable region and a portion of the | 37 |
| | constant region of the heavy chain | |
| | Amino acid sequence of the variable region of the heavy chain | 38 |
| | Nucleotide sequence encoding the variable region and a portion of the | 39 |
| | constant region of the light chain | |
| | Amino acid sequence of the variable region of the light chain | 40 |
| 2.70 | Nucleotide sequence encoding the variable region and a portion of the | 41 |
| | constant region of the heavy chain | |
| | Amino acid sequence of the variable region of the heavy chain | 42 |
| | Nucleotide sequence encoding the variable region and a portion of the | 43 |
| | constant region of the light chain | |
| | Amino acid sequence of the variable region of the light chain | 44 |
| 2.76 | Nucleotide sequence encoding the variable region and a portion of the | 45 |
| | constant region of the heavy chain | |
| | Amino acid sequence of the variable region of the heavy chain | 46 |
| | Nucleotide sequence encoding the variable region and a portion of the | 47 |
| | constant region of the light chain | |
| | Amino acid sequence of the variable region of the light chain | 48 |
| 2.70.2 | Nucleotide sequence encoding the variable region and a portion of the | 49 |
| | constant region of the heavy chain | |
| | Amino acid sequence of the variable region and a portion of the | 50 |
| | constant region of the heavy chain | |
| | Nucleotide sequence encoding the variable region and a portion of the | 51 |
| | constant region of the light chain | |
| | Amino acid sequence of the variable region and a portion of the | 52 |
| | constant region of the light chain | |

### Definitions

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “TIM-1” refers to T cell, immunoglobulin domain and mucin domain 1. In one embodiment, TIM-1 refers to a polypeptide comprising the amino acid sequence of SEQ ID NO: 50.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise human heavy chain immunoglobulin molecules and human kappa light chain immunoglobulin molecules, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides described herein can be either sense or antisense oligonucleotides.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as upstream sequences; sequence regions on the DNA strand having the same sequence as the RNA and which are 3′ to the 3′ end of the RNA transcript are referred to as downstream sequences.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, ed., Oxford University Press, Oxford England (1991)); Stec et al., U.S. Pat. No. 5,151,510; Uhlmann and Peyman, Chemical Reviews 90:543 (1990), the disclosures of which are hereby incorporated by reference. An oligonucleotide can include a label for detection, if desired.

The term “operably linked” as used herein refers to positions of components so described are in a relationship permitting them to function in their intended manner. A control sequence operably linked to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term control sequences is intended to include, at a minimum, all components whose presence is essential for expression and processing, and can also include additional components whose presence is advantageous, for example, leader sequences and fusion partner sequences.

The term “selectively hybridize” referred to herein means to detectably and specifically bind. Polynucleotides, oligonucleotides and fragments thereof described herein selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments described herein and a nucleic acid sequence of interest will be at least 80%, and more typically with preferably increasing homologies of at least 85%, 90%, 95%, 99%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA.”

The following terms are used to describe the sequence relationships between two or more polynucleotide or amino acid sequences: “reference sequence,” “comparison window,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a comparison window to identify and compare local regions of sequence similarity. A “comparison window,” as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Natl. Acad. Sci. (U.S.A.), 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term percentage of sequence identity is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides described herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the invention described herein, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al., Science, 253:164 (1991). Thus, the foregoing examples demonstrate that those of skill in the art can recognize sequence motifs and structural conformations that may be used to define structural and functional domains described herein.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature, 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a TIM-1, under suitable binding conditions, (2) ability to block appropriate TIM-1 binding, or (3) ability to inhibit the growth and/or survival of TIM-1 expressing cells in vitro or in vivo. Typically, polypeptide analogs comprise a conservative amino acid substitution (or addition or deletion) with respect to the naturally occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compounds are termed peptide mimetics or peptidomimetics. Fauchere, J. Adv. Drug Res., 15:29 (1986); Veber and Freidinger, TINS, p. 392 (1985); and Evans et al., J. Med. Chem., 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch, Ann. Rev. Biochem., 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a bispecific or bifunctional antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a “F(ab′)₂” fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites.

“Fab” when used herein refers to a fragment of an antibody which comprises the constant domain of the light chain and the CH1 domain of the heavy chain.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

The term “pharmaceutical agent” or “drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

“Active” or “activity” in regard to a TIM-1 polypeptide refers to a portion of a TIM-1 polypeptide which has a biological or an immunological activity of a native TIM-1 polypeptide. “Biological” when used herein refers to a biological function that results from the activity of the native TIM-1 polypeptide. A preferred biological activity includes, for example, regulation of cellular growth.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

“Mammal” when used herein refers to any animal that is considered a mammal. Preferably, the mammal is human.

“Liposome” when used herein refers to a small vesicle that may be useful for delivery of drugs that may include the TIM-1 polypeptide described herein or antibodies to such a TIM-1 polypeptide to a mammal.

The term “patient” includes human and veterinary subjects.

### Antibody Structure

The basic whole antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable domain of about 100 to 110 or more amino acids primarily responsible for antigen recognition. The carboxy-terminal portion of each chain defines a constant region primarily responsible for effector function. Human light chains are classified as kappa and lambda light chains. Human heavy chains are classified as mu, delta, gamma, alpha, or epsilon, and define the antibody's isotype as IgM, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2d ed. Raven Press, N.Y. (1989)) (incorporated by reference in its entirety for all purposes). The variable regions of each light/heavy chain pair form the antibody binding site.

The variable domains all exhibit the same general structure of relatively conserved framework regions (FR) joined by three hyper variable regions, also called complementarity determining regions or CDRs. The CDRs from the heavy and light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each region is in accordance with the definitions of Kabat, Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79: 315-321 (1990), Kostelny et al., J. Immunol. 148:1547-1553 (1992). Bispecific antibodies do not exist in the form of fragments having a single binding site (e.g., Fab, Fab′, and Fv).

It will be appreciated that such bifunctional or bispecific antibodies are contemplated and encompassed by the invention. A bispecific single chain antibody with specificity to TIM-1 and to the CD3 antigen on cytotoxic T lymphocytes can be used to direct these T cells to tumor cells expressing TIM-1 and cause apoptosis and eradication of the tumor. Two bispecific scFv constructs for this purpose are described herein. The scFv components specific for TIM-1 can be derived from anti-TIM-1 antibodies described herein. In some embodiments, the anti-TIM-1 antibody components disclosed in Tables 4 and 5 can be used to generate a biologically active scFv directed against TIM-1. In a preferred embodiment, the scFv components are derived from mAb 2.70. The anti-CD3 scFv component of the therapeutic bispecific scFv was derived from a sequence deposited in Genbank (accession number CAE85148). Alternative antibodies known to target CD3 or other T cell antigens may similarly be effective in treating malignancies when coupled with anti-TIM-1, whether on a single-chain backbone or a full IgG.

### Human Antibodies and Humanization of Antibodies

Embodiments of the invention described herein contemplate and encompass human antibodies. Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a mammal other than a rodent.

### Human Antibodies

The ability to clone and reconstruct megabase-sized human loci in YACs and to introduce them into the mouse germline provides a powerful approach to elucidating the functional components of very large or crudely mapped loci as well as generating useful models of human disease. An important practical application of such a strategy is the “humanization” of the mouse humoral immune system. Introduction of human immunoglobulin (Ig) loci into mice in which the endogenous Ig genes have been inactivated offers the opportunity to develop human antibodies in the mouse. Fully human antibodies are expected to minimize the immunogenic and allergic responses intrinsic to mouse or mouse-derivatized Mabs and thus to increase the efficacy and safety of the antibodies administered to humans. The use of fully human antibodies can be expected to provide a substantial advantage in the treatment of chronic and recurring human diseases, such as inflammation, autoimmunity, and cancer, which require repeated antibody administrations.

One approach toward this goal was to engineer mouse strains deficient in mouse antibody production with large fragments of the human Ig loci in anticipation that such mice would produce a large repertoire of human antibodies in the absence of mouse antibodies. This general strategy was demonstrated in connection with our generation of the first XenoMouse® strains as published in 1994. See Green et al., Nature Genetics 7:13-21 (1994). The XenoMouse® strains were engineered with yeast artificial chromosomes (YACs) containing 245 kb and 190 kb-sized germline configuration fragments of the human heavy chain locus and kappa light chain locus, respectively, which contained core variable and constant region sequences. Id. The XENOMOUSE® strains are available from Abgenix, Inc. (Fremont, Calif.). Greater than approximately 80% of the human antibody repertoire has been introduced through introduction of megabase sized, germline configuration YAC fragments of the human heavy chain loci and kappa light chain loci, respectively, to produce XenoMouse® mice.

The production of the XENOMOUSE® is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3,068,506 B2, and 3,068,507 B2. See also Mendez et al., Nature Genetics 15:146-156 (1997) and Green and Jakobovits, J. Exp. Med. 188:483-495 (1998). See also European Patent No. EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

Alternative approaches have utilized a “minilocus” approach, in which an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. Nos. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against TIM-1 in order to vitiate concerns and/or effects of human anti-mouse antibody (HAMA) or HACA response.

### Humanization and Display Technologies

Antibodies with reduced immunogenicity can be generated using humanization and library display techniques. It will be appreciated that antibodies can be humanized or primatized using techniques well known in the art. See e.g., Winter and Harris, Immunol Today 14:43-46 (1993) and Wright et al., Crit, Reviews in Immunol. 12:125-168 (1992). The antibody of interest can be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439 (1987) and J. Immunol. 139:3521 (1987)). mRNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, an expression library is made and screened to isolate the sequence of interest encoding the variable region of the antibody is then fused to human constant region sequences. The sequences of human constant regions genes can be found in Kabat et al., “Sequences of Proteins of Immunological Interest,” N.I.H. publication no. 91-3242 (1991). Human C region genes are readily available from known clones. The choice of isotype will be guided by the desired effector functions, such as complement fixation, or activity in antibody-dependent cellular cytotoxicity. Preferred isotypes are IgG1, IgG2 and IgG4. Either of the human light chain constant regions, kappa or lambda, can be used. The chimeric, humanized antibody is then expressed by conventional methods. Expression vectors include plasmids, retroviruses, YACs, EBV derived episomes, and the like.

Antibody fragments, such as Fv, F(ab′)₂ and Fab can be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama et al., Mol. Cell. Bio. 3:280 (1983)), Rous sarcoma virus LTR (Gorman et al., P.N.A.S. 79:6777 (1982)), and moloney murine leukemia virus LTR (Grosschedl et al., Cell 41:885 (1985)). Also, as will be appreciated, native Ig promoters and the like can be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau, PNAS USA 94:4937-4942 (1997) (ribosomal display), Parmley and Smith, Gene 73:305-318 (1988) (phage display), Scott, TIBS 17:241-245 (1992), Cwirla et al., PNAS USA 87:6378-6382 (1990), Russel et al., Nucl. Acids Res. 21:1081-1085 (1993), Hoganboom et al., Immunol. Reviews 130:43-68 (1992), Chiswell and McCafferty, TIBTECH 10:80-84 (1992), and U.S. Pat. No. 5,733,743. If display technologies are utilized to produce antibodies that are not human, such antibodies can be humanized as described above.

Using these techniques, antibodies can be generated to TIM-1 expressing cells, TIM-1 itself, forms of TIM-1, epitopes or peptides thereof, and expression libraries thereto (see e.g. U.S. Pat. No. 5,703,057) which can thereafter be screened as described above for the activities described above.

### Antibody Therapeutics

In certain respects, it can be desirable in connection with the generation of antibodies as therapeutic candidates against TIM-1 that the antibodies be capable of fixing complement and participating in complement-dependent cytotoxicity (CDC). Such antibodies include, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see, e.g., U.S. Pat. No. 4,816,397), cell-cell fusion techniques (see, e.g., U.S. Pat. Nos. 5,916,771 and 6,207,418), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, the TIM-1 antibody discussed herein is a human anti-TIM-1 IgG2 antibody. If such antibody possessed desired binding to the TIM-1 molecule, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC.

### Design and Generation of Other Therapeutics

Due to their association with renal and pancreatic tumors, head and neck cancer, ovarian cancer, gastric (stomach) cancer, melanoma, lymphoma, prostate cancer, liver cancer, breast cancer, lung cancer, renal cancer, bladder cancer, colon cancer, esophageal cancer, and brain cancer, antineoplastic agents comprising anti-TIM-1 antibodies are contemplated and encompassed by the invention.

Moreover, based on the activity of the antibodies that are produced and characterized herein with respect to TIM-1, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it can be possible to sidestep the dependence on complement for cell killing through the use of bispecifics, immunotoxins, or radiolabels, for example.

For example, in connection with bispecific antibodies, bispecific antibodies can be generated that comprise (i) two antibodies one with a specificity to TIM-1 and another to a second molecule that are conjugated together, (ii) a single antibody that has one chain specific to TIM-1 and a second chain specific to a second molecule, or (iii) a single chain antibody that has specificity to TIM-1 and the other molecule. Such bispecific antibodies can be generated using techniques that are well known for example, in connection with (i) and (ii) see, e.g., Fanger et al., Immunol Methods 4:72-81 (1994) and Wright and Harris, supra and in connection with (iii) see, e.g., Traunecker et al., Int. J. Cancer (Suppl.) 7:51-52 (1992). In each case, the second specificity can be made to the heavy chain activation receptors, including, without limitation, CD16 or CD64 (see, e.g., Deo et al., 18:127 (1997)) or CD89 (see, e.g., Valerius et al., Blood 90:4485-4492 (1997)). Bispecific antibodies prepared in accordance with the foregoing would be likely to kill cells expressing TIM-1, and particularly those cells in which the TIM-1 antibodies described herein are effective.

With respect to immunotoxins, antibodies can be modified to act as immunotoxins utilizing techniques that are well known in the art. See, e.g., Vitetta, Immunol Today 14:252 (1993). See also U.S. Pat. No. 5,194,594. In connection with the preparation of radiolabeled antibodies, such modified antibodies can also be readily prepared utilizing techniques that are well known in the art. See, e.g., Junghans et al., in Cancer Chemotherapy and Biotherapy 655-686 (2d ed., Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing TIM-1, and particularly those cells in which the antibodies described herein are effective.

In connection with the generation of therapeutic peptides, through the utilization of structural information related to TIM-1 and antibodies thereto, such as the antibodies described herein (as discussed below in connection with small molecules) or screening of peptide libraries, therapeutic peptides can be generated that are directed against TIM-1. Design and screening of peptide therapeutics is discussed in connection with Houghten et al., Biotechniques 13:412-421 (1992), Houghten, PNAS USA 82:5131-5135 (1985), Pinalla et al., Biotechniques 13:901-905 (1992), Blake and Litzi-Davis, BioConjugate Chem. 3:510-513 (1992). Immunotoxins and radiolabeled molecules can also be prepared, and in a similar manner, in connection with peptidic moieties as discussed above in connection with antibodies.

Assuming that the TIM-1 molecule (or a form, such as a splice variant or alternate form) is functionally active in a disease process, it will also be possible to design gene and antisense therapeutics thereto through conventional techniques. Such modalities can be utilized for modulating the function of TIM-1. In connection therewith the antibodies, as described herein, facilitate design and use of functional assays related thereto. A design and strategy for antisense therapeutics is discussed in detail in International Patent Application No. WO 94/29444. Design and strategies for gene therapy are well known. However, in particular, the use of gene therapeutic techniques involving intrabodies could prove to be particularly advantageous. See, e.g., Chen et al., Human Gene Therapy 5:595-601 (1994) and Marasco, Gene Therapy 4:11-15 (1997). General design of and considerations related to gene therapeutics is also discussed in International Patent Application No. WO 97/38137.

Small molecule therapeutics can also be envisioned. Drugs can be designed to modulate the activity of TIM-1, as described herein. Knowledge gleaned from the structure of the TIM-1 molecule and its interactions with other molecules, as described herein, such as the antibodies described herein, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of TIM-1. Such structures can be synthesized chemically or expressed in biological systems. This approach has been reviewed in Capsey et al., Genetically Engineered Human Therapeutic Drugs (Stockton Press, NY (1988)). Further, combinatorial libraries can be designed and synthesized and used in screening programs, such as high throughput screening efforts.

### TIM-1 Agonists and Antagonists

Embodiments of the invention described herein also pertain to variants of a TIM-1 protein that function as either TIM-1 agonists (mimetics) or as TIM-1 antagonists. Variants of a TIM-1 protein can be generated by mutagenesis, e.g., discrete point mutation or truncation of the TIM-1 protein. An agonist of the TIM-1 protein can retain substantially the same, or a subset of, the biological activities of the naturally occurring form of the TIM-1 protein. An antagonist of the TIM-1 protein can inhibit one or more of the activities of the naturally occurring form of the TIM-1 protein by, for example, competitively binding to a downstream or upstream member of a cellular signaling cascade which includes the TIM-1 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the TIM-1 protein.

Variants of the TIM-1 protein that function as either TIM-1 agonists (mimetics) or as TIM-1 antagonists can be identified by screening combinatorial libraries of mutants, e.g., truncation mutants, of the TIM-1 protein for protein agonist or antagonist activity. In one embodiment, a variegated library of TIM-1 variants is generated by combinatorial mutagenesis at the nucleic acid level and is encoded by a variegated gene library. A variegated library of TIM-1 variants can be produced by, for example, enzymatically ligating a mixture of synthetic oligonucleotides into gene sequences such that a degenerate set of potential TIM-1 sequences is expressible as individual polypeptides, or alternatively, as a set of larger fusion proteins (e.g., for phage display) containing the set of TIM-1 sequences therein. There are a variety of methods which can be used to produce libraries of potential TIM-1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TIM-1 variant sequences. Methods for synthesizing degenerate oligonucleotides are known in the art (see, e.g., Narang, Tetrahedron 39:3 (1983); Itakura et al., Annu. Rev. Biochem. 53:323 (1984); Itakura et al., Science 198:1056 (1984); Ike et al., Nucl. Acid Res. 11:477 (1983).

### Radioimmuno & Immunochemotherapeutic Antibodies

Cytotoxic chemotherapy or radiotherapy of cancer is limited by serious, sometimes life-threatening, side effects that arise from toxicities to sensitive normal cells because the therapies are not selective for malignant cells. Therefore, there is a need to improve the selectivity. One strategy is to couple therapeutics to antibodies that recognize tumor-associated antigens. This increases the exposure of the malignant cells to the ligand-targeted therapeutics but reduces the exposure of normal cells to the same agent. See Allen, Nat. Rev. Cancer 2(10):750-63 (2002).

The TIM-1 antigen is one of these tumor-associated antigens, as shown by its specific expression on cellular membranes of tumor cells by FACS and IHC. Therefore one embodiment of the invention is to use monoclonal antibodies directed against the TIM-1 antigen coupled to cytotoxic chemotherapic agents or radiotherapic agents as anti-tumor therapeutics.

Radiolabels are known in the art and have been used for diagnostic or therapeutic radioimmuno conjugates. Examples of radiolabels includes, but are not limited to, the following: radioisotopes or radionuclides (e.g., 3H, 14C, 15N, 35S, 90Y, 99Tc, 111In, 125I, 131I, 177Lu, Rhenium-186, Rhenium-188, Samarium-153, Copper-64, Scandium-47). For example, radionuclides which have been used in radioimmunoconjugate guided clinical diagnosis include, but are not limited to: 131 I, 125 I, 123 I, 99 Tc, 67 Ga, as well as 111 In. Antibodies have also been labeled with a variety of radionuclides for potential use in targeted immunotherapy (see Peirersz et al., 1987). Monoclonal antibody conjugates have also been used for the diagnosis and treatment of cancer (e.g., Immunol. Cell Biol. 65:111-125). These radionuclides include, for example, 188 Re and 186 Re as well as 90 Y, and to a lesser extent 199 Au and 67 Cu. I-(131) have also been used for therapeutic purposes. U.S. Pat. No. 5,460,785 provides a listing of such radioisotopes. Radiotherapeutic chelators and chelator conjugates are known in the art. See U.S. Pat. Nos. 4,831,175, 5,099,069, 5,246,692, 5,286,850, and 5,124,471.

Immunoradiopharmaceuticals utilizing anti-TIM-1 antibodies can be prepared utilizing techniques that are well known in the art. See, e.g., Junghans et al., in Cancer Chemotherapy and Biotherapy 655-686 (2d ed., Chafner and Longo, eds., Lippincott Raven (1996)), U.S. Pat. Nos. 4,681,581, 4,735,210, 5,101,827, RE 35,500, 5,648,471, and 5,697,902.

Cytotoxic immunoconjugates are known in the art and have been used as therapeutic agents. Such immunoconjugates may for example, use maytansinoids (U.S. Pat. No. 6,441,163), tubulin polymerization inhibitor, auristatin (Mohammad et al., Int. J. Oncol. 15(2):367-72 (1999); Doronina et al., Nature Biotechnology 21(7):778-784 (2003)), dolastatin derivatives (Ogawa et al., Toxicol Lett. 121(2):97-106 (2001); 21(3)778-784), Mylotarg® (Wyeth Laboratories, Philadelphia, Pa.); maytansinoids (DM1), taxane or mertansine (ImmunoGen Inc.). Immunotoxins utilizing anti-TIM-1 antibodies may be prepared by techniques that are well known in the art. See, e.g., Vitetta, Immunol Today 14:252 (1993); U.S. Pat. No. 5,194,594.

Bispecific antibodies may be generated using techniques that are well known in the art for example, see, e.g., Fanger et al., Immunol Methods 4:72-81 (1994); Wright and Harris, supra; Traunecker et al., Int. J. Cancer (Suppl.) 7:51-52 (1992). In each case, the first specificity is to TIM-1, the second specificity may be made to the heavy chain activation receptors, including, without limitation, CD16 or CD64 (see, e.g., Deo et al., 18:127 (1997)) or CD89 (see, e.g., Valerius et al., Blood 90:4485-4492 (1997)). Bispecific antibodies prepared in accordance with the foregoing would kill cells expressing TIM-1.

Depending on the intended use of the antibody, i.e., as a diagnostic or therapeutic reagent, radiolabels are known in the art and have been used for similar purposes. For example, radionuclides which have been used in clinical diagnosis include, but are not limited to: ¹³¹I, ¹²⁵I, ¹²³I, ⁹⁹Tc, ⁶⁷Ga, as well as ¹¹¹In. Antibodies have also been labeled with a variety of radionuclides for potential use in targeted immunotherapy. See Peirersz et al., (1987). Monoclonal antibody conjugates have also been used for the diagnosis and treatment of cancer. See, e.g., Immunol. Cell Biol. 65:111-125. These radionuclides include, for example, .sup.188 Re and .sup.186 Re as well as .sup.90 Y, and to a lesser extent .sup.199 Au and .sup.67 Cu. I-(131) have also been used for therapeutic purposes. U.S. Pat. No. 5,460,785 provides a listing of such radioisotopes.

Patents relating to radiotherapeutic chelators and chelator conjugates are known in the art. For example, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, and 5,124,471 of Gansow also relate to polysubstituted DTPA chelates.

Cytotoxic chemotherapies are known in the art and have been used for similar purposes. For example, U.S. Pat. No. 6,441,163 describes the process for the production of cytotoxic conjugates of maytansinoids and antibodies. The anti-tumor activity of a tubulin polymerization inhibitor, auristatin PE, is also known in the art. Mohammad et al., Int. J. Oncol. 15(2):367-72 (August 1999).

### Preparation of Antibodies

Briefly, XenoMouse® lines of mice were immunized with TIM-1 protein, lymphatic cells (such as B-cells) were recovered from the mice that express antibodies and were fused with a myeloid-type cell line to prepare immortal hybridoma cell lines, and such hybridoma cell lines were screened and selected to identify hybridoma cell lines that produce antibodies specific to TIM-1. Alternatively, instead of being fused to myeloma cells to generate hybridomas, the recovered B cells, isolated from immunized XenoMouse® lines of mice, with reactivity against TIM-1 (determined by e.g. ELISA with TIM-1-His protein), were then isolated using a TIM-1-specific hemolytic plaque assay. Babcook et al., Proc. Natl. Acad. Sci. USA, 93:7843-7848 (1996). In this assay, target cells such as sheep red blood cells (SRBCs) were coated with the TIM-1 antigen. In the presence of a B cell culture secreting the anti-TIM-1 antibody and complement, the formation of a plaque indicates specific TIM-1-mediated lysis of the target cells. Single antigen-specific plasma cells in the center of the plaques were isolated and the genetic information that encodes the specificity of the antibody isolated from single plasma cells.

Using reverse-transcriptase PCR, the DNA encoding the variable region of the antibody secreted was cloned and inserted into a suitable expression vector, preferably a vector cassette such as a pcDNA, more preferably the pcDNA vector containing the constant domains of immunoglobulin heavy and light chain. The generated vector was then be transfected into host cells, preferably CHO cells, and cultured in conventional nutrient media modified as appropriate for inducing promoters, selecting transformants, or amplifying the genes encoding the desired sequences.

In general, antibodies produced by the above-mentioned cell lines possessed fully human IgG2 heavy chains with human kappa light chains. The antibodies possessed high affinities, typically possessing Kd's of from about 10-6 through about 10-11 M, when measured by either solid phase and solution phase. These mAbs can be stratified into groups or “bins” based on antigen binding competition studies, as discussed below.

As will be appreciated, antibodies, as described herein, can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive TIM-1 binding properties.

### Therapeutic Administration and Formulations

The compounds of the invention are formulated according to standard practice, such as prepared in a carrier vehicle. The term “pharmacologically acceptable carrier” means one or more organic or inorganic ingredients, natural or synthetic, with which the mutant proto-oncogene or mutant oncoprotein is combined to facilitate its application. A suitable carrier includes sterile saline although other aqueous and non-aqueous isotonic sterile solutions and sterile suspensions known to be pharmaceutically acceptable are known to those of ordinary skill in the art. In this regard, the term “carrier” encompasses liposomes and the antibody (See Chen et al., Anal. Biochem. 227: 168-175 (1995) as well as any plasmid and viral expression vectors.

Any of the novel polypeptides of this invention may be used in the form of a pharmaceutically acceptable salt. Suitable acids and bases which are capable of forming salts with the polypeptides of the present invention are well known to those of skill in the art, and include inorganic and organic acids and bases.

A compound of the invention is administered to a subject in a therapeutically-effective amount, which means an amount of the compound which produces a medically desirable result or exerts an influence on the particular condition being treated. An effective amount of a compound of the invention is capable of ameliorating or delaying progression of the diseased, degenerative or damaged condition. The effective amount can be determined on an individual basis and will be based, in part, on consideration of the physical attributes of the subject, symptoms to be treated and results sought. An effective amount can be determined by one of ordinary skill in the art employing such factors and using no more than routine experimentation.

The compounds of the invention may be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the invention, by such means as depot injections or erodible implants. Localized delivery is particularly contemplated, by such means as delivery via a catheter to one or more arteries, such as the renal artery or a vessel supplying a localized tumor.

Biologically active anti-TIM-1 antibodies as described herein can be used in a sterile pharmaceutical preparation or formulation to reduce the level of serum TIM-1 thereby effectively treating pathological conditions where, for example, serum TIM-1 is abnormally elevated. Anti-TIM-1 antibodies preferably possess adequate affinity to potently suppress TIM-1 to within the target therapeutic range, and preferably have an adequate duration of action to allow for infrequent dosing. A prolonged duration of action will allow for less frequent and more convenient dosing schedules by alternate parenteral routes such as subcutaneous or intramuscular injection.

When used for in vivo administration, the antibody formulation must be sterile. This is readily accomplished, for example, by filtration through sterile filtration membranes, prior to or following lyophilization and reconstitution. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it is preferred that the therapist titer the dosage and modify the route of administration as required to obtain the optimal therapeutic effect. Typically, the clinician will administer antibody until a dosage is reached that achieves the desired effect. The progress of this therapy is easily monitored by conventional assays or by the assays described herein.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition can also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with physiologically acceptable carriers, excipients, or stabilizers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like can be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization can be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages can be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the therapeutic antibody until a dosage is reached that achieves the desired effect. The progress of this therapy is easily monitored by conventional assays or as described herein.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures can be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci 0.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

It is expected that the antibodies described herein will have therapeutic effect in treatment of symptoms and conditions resulting from TIM-1 expression. In specific embodiments, the antibodies and methods herein relate to the treatment of symptoms resulting from TIM-1 expression including symptoms of cancer. Further embodiments, involve using the antibodies and methods described herein to treat cancers, such as cancer of the lung, colon, stomach, kidney, prostrate, or ovary.

### Diagnostic Use

TIM-1 has been found to be expressed at low levels in normal kidney but its expression is increased dramatically in postischemic kidney. Ichimura et al., J. Biol. Chem. 273(7):4135-42 (1998). As immunohistochemical staining with anti-TIM-1 antibody shows positive staining of renal, kidney, prostate and ovarian carcinomas (see below), TIM-1 overexpression relative to normal tissues can serve as a diagnostic marker of such diseases.

Antibodies, including antibody fragments, can be used to qualitatively or quantitatively detect the expression of TIM-1 proteins. As noted above, the antibody preferably is equipped with a detectable, e.g., fluorescent label, and binding can be monitored by light microscopy, flow cytometry, fluorimetry, or other techniques known in the art. These techniques are particularly suitable if the amplified gene encodes a cell surface protein, e.g., a growth factor. Such binding assays are performed as known in the art.

In situ detection of antibody binding to the TIM-1 protein can be performed, for example, by immunofluorescence or immunoelectron microscopy. For this purpose, a tissue specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

### Epitope Mapping

The specific part of the protein immunogen recognized by an antibody may be determined by assaying the antibody reactivity to parts of the protein, for example an N terminal and C terminal half. The resulting reactive fragment can then be further dissected, assaying consecutively smaller parts of the immunogen with the antibody until the minimal reactive peptide is defined. Anti-TIM-1 mAb 2.70.2 was assayed for reactivity against overlapping peptides designed from the antigen sequence and was found to specifically recognize the amino acid sequence PLPRQNHE (SEQ ID NO:96) corresponding to amino acids 189-202 of the TIM-1 immunogen (SEQ ID NO:50). Furthermore using an alanine scanning technique, it has been determined that the second proline and the asparagine residues appear to be important for mAb 2.70.2 binding.

Alternatively, the epitope that is bound by the anti-TIM-1 antibodies of the invention may be determined by subjecting the TIM-1 immunogen to SDS-PAGE either in the absence or presence of a reduction agent and analyzed by immunoblotting. Epitope mapping may also be performed using SELDI. SELDI ProteinChip® (LumiCyte) arrays used to define sites of protein-protein interaction. TIM-1 protein antigen or fragments thereof may be specifically captured by antibodies covalently immobilized onto the PROTEINCHIP array surface. The bound antigens may be detected by a laser-induced desorption process and analyzed directly to determine their mass.

The epitope recognized by anti-TIM-1 antibodies described herein may be determined by exposing the PROTEINCHIP Array to a combinatorial library of random peptide 12-mer displayed on Filamentous phage (New England Biolabs). Antibody-bound phage are eluted and then amplified and taken through additional binding and amplification cycles to enrich the pool in favor of binding sequences. After three or four rounds, individual binding clones are further tested for binding by phage ELISA assays performed on antibody-coated wells and characterized by specific DNA sequencing of positive clones.

### EXAMPLES

The following examples, including the experiments conducted and results achieved are provided for illustrative purposes only and are not to be construed as limiting upon the invention described herein.

### Example 1

### Preparation of Monoclonal Antibodies that Bind TIM-1

The soluble extracellular domain of TIM-1 was used as the immunogen to stimulate an immune response in XenoMouse® animals. A DNA (CG57008-02), which encodes the amino acid sequence for the TIM-1 extracellular domain (minus the predicted N-terminal signal peptide) was subcloned to the baculovirus expression vector, pMelV5His (CuraGen Corp., New Haven, Conn.), expressed using the pBlueBac baculovirus expression system (Invitrogen Corp., Carlsbad, Calif.), and confirmed by Western blot analyses. The nucleotide sequence below encodes the polypeptide used to generate antibodies.

(SEQ ID NO: 53)
TCTGTAAAGGTTGGTGGAGAGGCAGGTCCATCTGTCACACTACCCTGCCA
CTACAGTGGAGCTGTCACATCAATGTGCTGGAATAGAGGCTCATGTTCTC
TATTCACATGCCAAAATGGCATTGTCTGGACCAATGGAACCCACGTCACC
TATCGGAAGGACACACGCTATAAGCTATTGGGGGACCTTTCAAGAAGGGA
TGTCTCTTTGACCATAGAAAATACAGCTGTGTCTGACAGTGGCGTATATT
GTTGCCGTGTTGAGCACCGTGGGTGGTTCAATGACATGAAAATCACCGTA
TCATTGGAGATTGTGCCACCCAAGGTCACGACTACTCCAATTGTCACAAC
TGTTCCAACCGTCACGACTGTTCGAACGAGCACCACTGTTCCAACGACAA
CGACTGTTCCAACGACAACTGTTCCAACAACAATGAGCATTCCAACGACA
ACGACTGTTCCGACGACAATGACTGTTTCAACGACAACGAGCGTTCCAAC
GACAACGAGCATTCCAACAACAACAAGTGTTCCAGTGACAACAACGGTCT
CTACCTTTGTTCCTCCAATGCCTTTGCCCAGGCAGAACCATGAACCAGTA
GCCACTTCACCATCTTCACCTCAGCCAGCAGAAACCCACCCTACGACACT
GCAGGGAGCAATAAGGAGAGAACCCACCAGCTCACCATTGTACTCTTACA
CAACAGATGGGAATGACACCGTGACAGAGTCTTCAGATGGCCTTTGGAAT
AACAATCAAACTCAACTGTTCCTAGAACATAGTCTACTG

The amino acid sequence encoded thereby is as follows:

(SEQ ID NO: 50)
SVKVGGEAGPSVTLPCHYSGAVTSMCWNRGSCSLFTCONGIVWTNGTHVT
YRKDTRYKLLGDLSRRDVSLTIENTAVSDSGVYCCRVEHRGWFNDMKITV
SLEIVPPKVTTTPIVTTVPTVTTVRTSTTVPTTTTVPTTTVPTTMSIPTT
TTVPTTMTVSTTTSVPTTTSIPTTTSVPVTTTVSTFVPPMPLPRQNHEPV
ATSPSSPOPAETHPTTLOGAIRREPTSSPLYSYTTDGNDTVTESSDGLWN
NNQTQLFLEHSLL

To facilitate purification of recombinant TIM-1, the expression construct can incorporate coding sequences for the V5 binding domain V5 and a HIS tag. Fully human IgG2 and IgG4 monoclonal antibodies (mAb), directed against TIM-1 were generated from human antibody-producing XenoMouse® strains engineered to be deficient in mouse antibody production and to contain the majority of the human antibody gene repertoire on megabase-sized fragments from the human heavy and kappa light chain loci as previously described in Yang et al., Cancer Res. (1999). Two XenoMouse® strains, an hIgG2 (xmg-2) strain and an IgG4 (3C-1) strain, were immunized with the TIM-1 antigen (SEQ ID NO: 50). Both strains responded well to immunization (Tables 2 and 3).

TABLE 2
Serum titer of XENOMOUSE ® hIgG₂ strain
immunized with TIM-1 antigen.
Group 1: 5 mice (hIgG₂ strain); mode of immunization = footpad

Reactivity to TIM-1
Titers via hIgG

| Mouse ID | Bleed After 4 inj. | Bleed After 6 inj. |
|---|---|---|
| M716-1 | 600,000 | 600,000 |
| M716-2 | 600,000 | 500,000 |
| M716-3 | 200,000 | 400,000 |
| M716-4 | 300,000 | 200,000 |
| M716-5 | 400,000 | 400,000 |
| Negative Control | 75 | 110 |
| Positive Control | — | 600,000 |

TABLE 3
Serum titer of XENOMOUSE ® IgG₄ strain
immunized with TIM-1 antigen
Group 2: 5 mice (IgG₄ strain); mode of immunization = footpad

Reactivity to TIM-1
Titers via hIgG

| Mouse ID | Bleed After 4 inj. | Bleed After 6 inj. |
|---|---|---|
| M326-2 | 15,000 | 73,000 |
| M326-3 | 7,500 | 60,000 |
| M329-1 | 27,000 | 30,000 |
| M329-3 | 6,500 | 50,000 |
| M337-1 | 2,500 | 16,000 |
| Negative Control | <100 | 90 |
| Positive Control | — | 600,000 |

Hybridoma cell lines were generated from the immunized mice. Selected hybridomas designated 1.29, 1.37, 2.16, 2.17, 2.24, 2.45, 2.54 2.56, 2.59, 2.61, 2.70, and 2.76 (and subclones thereof) were further characterized. The antibodies produced by cell lines 1.29 and 1.37 possess fully human IgG2 heavy chains with human kappa light chains while those antibodies produced by cell lines 2.16, 2.17, 2.24, 2.45, 2.54 2.56, 2.59, 2.61, 2.70, and 2.76 possess fully human IgG4 heavy chains with human kappa light chains.

The amino acid sequences of the heavy chain variable domain regions of twelve anti-TIM-1 antibodies with their respective germline sequences are shown in Table 4 below. The corresponding light chain variable domain regions amino acid sequence is shown in Table 5 below. “X” indicates any amino acid, preferably the germline sequence in the corresponding amino acid position. The CDRs (CDR1, CDR2, and CDR3) and FRs (FR1, FR2, and FR3) in the immunoglobulins are shown under the respective column headings.

TABLE 4
Heavy Chain Analysis

| | SEQ | | | | | | | | |
|---|---|---|---|---|---|---|---|---|---|
| | ID | | | | | | | | |
| mAb | NO: | D | FR1 | CDR1 | FR2 | CDR2 | FR3 | CDR3 | J |
| | 55 | Germline | QVQLVESGGGVVQP | GFTFSSYGMH | WVRQAPGKG | VIWYDGSNKYYADSVKG | RFTISRDNSKNTLYLQMN | XXDY | WGQGTLVTVSSA |
| | | | GRSLRLSCAAS | | LEWVA | | SLRAEDTAVYYCAR | | |
| 2.54 | 26 | VH3-33/--/JH4b | QVQLEQSGGGVVQP | GFTFTNYGLH | WVRQAPGKG | VIWYDGSHKFYADSVKG | RFTISRDNSKNTLFLQMN | DLDY | WGQGTLVTVSSA |
| | | | GRSLRLSCAAS | | LDWVA | | SLRAEDTAVYYCTR | | |
| | 56 | Germline | QVQLVESGGGVVQP | GFTFSSYGMH | WVRQAPGKG | VIWYDGSNKYYADSVKG | RFTISRDNSKNTLYLQMN | XXYDSSXXXYGMDV | WGQGTTVTVSSA |
| | | | GRSLRLSCAAS | | LEWVA | | SLRAEDTAVYYCAX | | |
| 2.76 | 46 | VH3-33/D3-22/JH6b | XXXXEQSGGGVVQP | GFTFSSYGMY | WVRQAPGKG | VIWYDGSNKYYADSVKG | RFTISRDNSKNTLYLQMN | DFYDSSRYHYGMDV | WGQGTTVTVSSA |
| | | | GRSLRLSCAAS | | LEWVA | | SLRAEDTAVYYCAR | | |
| | 57 | Germline | QVQLQESGPGLVKP | GGSISSGGYYWS | WIRQHPGKG | YIYYSGSTYYNPSLKS | RVTISVDTSKNQFSLKLS | XXXXSSSWYXXFDY | WGQGTLVTVSSA |
| | | | SQTLSLTCTVS | | LEWIG | | SVTAADTAVYYCAR | | |
| 2.59 | 34 | VH4-31/D6-13/JH4b | XXXXXQSGPRLVKP | GGSISSDGYYWS | WIRQHPGKG | YIYYSGSTFYNPSLKS | RVAISVDTSKNQFSLKLS | ESPHSSNWYSGFDC | WGQGTLVTVSSA |
| | | | SQTLSLTCTVS | | LEWIG | | SVTAADTAVYYCAR | | |
| | 58 | Germline | QVQLVESGGGVVQP | GFTFSSYGMH | WVRQAPGKG | VIWYDGSNKYYADSVKG | RFTISRDNSKNTLYLQMN | DYYDSSXXXXXFDY | WGQGTLVTVSSA |
| | | | GRSLRLSCAAS | | LEWVA | | SLRAEDTAVYYCAR | | |
| 2.70 | 42 | VH3-33/D3-22/JH4b | QVQLVESGGGVVQP | GFIFSRYGMH | WVRQAPGKG | VIWYDGSNKLYADSVKG | RFTISRDNSKNTLYLQMN | DYYDNSRHHWGFDY | WGQGTLVTVSSA |
| | | | GRSLRLSCAAS | | LKWVA | | SLRAEDTAVYYCAR | | |
| 2.24 | 18 | | QVQLEQSGGGVVQP | GFTFSRYGMH | WVRQAPGKG | VIWYDGSNKLYADSVKG | RFTISRDNSKNTLYLQMN | DYYDNSRHHWGFDY | WGQGTLVTVSSA |
| | | | GRSLRLSCAAS | | LKWVA | | SLRAEDTAVYYCAR | | |
| 2.61 | 38 | | QVQLVEAGGGVVQP | GFTFRSYGMH | WVRQAPGKG | VIWYDGSNKYYTDSVKG | RFTISRDNSKNTLYLQMN | DYYDNSRHHWGFDY | WGQGTLVTVSSA |
| | | | GRSLRLSCAAS | | LKWVA | | SLRAEDTAVYYCVR | | |
| 2.56 | 30 | | QVQLVESGGGVVQP | GFTFSSYGMH | WVRQAPGKG | VIWYDGSHKYYADSVKG | RFTISRDNSKNTLYLQMN | DYYDTSRHHWGFDC | WGQGTLVTVSSA |
| | | | GRSLRLSCAAS | | LEWVA | | SLRAEDTAVYYSAR | | |
| | 59 | Germline | EVQLVESGGGLVKP | GFTFSNAWMS | WVRQAPGKG | RIKSKTDGGTTDYAAPVKG | RFTISRDDSKNTLYLQMN | XDXXXDY | WGQGTLVTVSSA |
| | | | GGSLRLSCAAS | | LEWVG | | SLKTEDTAVYYCTX | | |
| 2.16 | 10 | VH3-15/D3-16/JH4b | XXXXEQSGGGVVKP | GFTFSNAWMT | WVRQAPGKG | RIKRRTDGGTTDYAAPVKG | RFTISRDDSKNTLYLQMN | VDNDVDY | WGQGTLVTVSSA |
| | | | GGSLRLSCAAS | | LEWVG | | NLKNEDTAVYYCTS | | |
| | 60 | Germline | QVQLQESGPGLVKP | GGSVSSGGYYWS | WIRQPPGKG | YIYYSGSTNYNPSLKS | RVTISVDTSKNQFSLKLS | XXXWXXXFDY | WGQGTLVTVSSA |
| | | | SETLSLTCTVS | | LEWIG | | SVTAADTAVYYCAR | | |
| 1.29 | 2 | VH4-61/D1-7/JH4b | QVQLQESGPGLVKP | GGSVSSGGYYWS | WIRQPPGKG | FIYYTGSTNYNPSLKS | RVSISVDTSKNQFSLKLS | DYDWSFHFDY | WGQGTLVTVSSA |
| | | | SETLSLTCTVS | | LEWIG | | SVTAADAAVYYCAR | | |
| | 61 | Germline | EVQLVESGGGLVKP | GFTFSNAWMS | WVRQAPGKG | RIKSKTDGGTTDYAAPVKG | RFTISRDDSKNTLYLQMN | XXXSGDY | WGQGTLVTVSSA |
| | | | GGSLRLSCAAS | | LEWVG | | SLKTEDTAVYYCTT | | |
| 2.45 | 22 | VH3-15/D6-19/JH4b | XXXXXQSGGGLVKP | GFTFSNAWMT | WVRQAPGKG | RIKRKTDGGTTDYAAPVKG | RFTISRDDSENTLYLQMN | VDNSGDY | WGQGTLVTVSSA |
| | | | GGSLRLSCAAS | | LEWVG | | SLETEDTAVYYCTT | | |
| | 62 | Germline | EVQLVESGGGLVQP | GFTFSSYWMS | WVRQAPGKG | NIKQDGSEKYYVDSVKG | RFTISRDNAKNSLYLQMN | XDY | WGQGTLVTVSSA |
| | | | GGSLRLSCAAS | | LEWVA | | SLRAEDTAVYYCAR | | |
| 1.37 | 6 | VH3-7/--/JH4b | EVQLVESGGGLVQP | GFTFTNYWMS | WVRQAPGKG | NIQQDGSEKYYVDSVRG | RFTISRDNAKNSLYLQMN | WDY | WGQGTLVTVSSA |
| | | | GGSLRLSCAAS | | LEWVA | | SLRAEDSAVYYCAR | | |
| | 63 | Germline | EVQLVESGGGLVQP | GFTFSSYSMN | WVRQAPGKG | YISSSSSTIYYADSVKG | RFTISRDNAKNSLYLQMN | XFDY | WGQGTLVTVSSA |
| | | | GGSLRLSCAAS | | LEWVS | | SLRDEDTAVYYCAX | | |
| 2.17 | 14 | VH3-48/--/JH4b | QVQLEQSGGGLVQP | GFTFSTYSMN | WVRQAPGKG | YIRSSTSTIYYAESLKG | RFTISSDNAKNSLYLQMN | DFDY | WGQGTLVTVSSA |
| | | | GGSLRLSCAAS | | LEWVS | | SLRDEDTAVYYCAR | | |

TABLE 5
Light Chain Analysis

| | SEQ ID | | | | | | | | |
|---|---|---|---|---|---|---|---|---|---|
| mAb | NO: | J | FR1 | CDR1 | FR2 | CDR2 | FR3 | CDR3 | J |
| | 64 | Germline | EIVLTQSPGTLSLS | RASQSVSSSYLA | WYQQKPGQAPR | GASSRAT | GIPDRFSGSGSGTDFTLTISRL | QQYGSSXXLT | FGGGTKVEIKR |
| | | | PGERATLSC | | LLIY | | EPEDFAVYYC | | |
| 2.54 | 28 | A27/JK4 | ETQLTQSPGTLSLS | RASQSVSNNYLA | WYQQKPGQAPR | GASSRAT | GIPDRFSGSGSGTDFTLTISRL | QQYGSSLPLT | FGGGTKVEIKR |
| | | | PGERVTLSC | | LLIY | | EPEDCAECYC | | |
| | 65 | Germline | DIVMTQSPLSLPVT | RSSQSLLHSNGYN | WYLQKPGQSPQ | LGSNRAS | GVPDRFSGSGSGTDFTLKISRV | MQALQTXXT | FGGGTKVEIKR |
| | | | PGEPASISC | YLD | LLIY | | EAEDVGVYYC | | |
| 2.16 | 12 | A3/JK4 | XXXLTQSPLSLPVT | RSSQSLLHSNGYN | WYLQKPGQSPQ | LGSNRAS | GVPDRFSGSGSGTDFTLKISRV | MQALQTPLT | FGGGTKVDIKR |
| | | | PGEPASISC | YLD | LLIY | | EAEDIGLYYC | | |
| 2.45 | 24 | | XXXXTQSPLSLPVT | RSSQSLLHSNGYN | WYLQKPGQSPQ | LGSNRAS | GVPDRFSGSGSGTDFTLKISRV | MQALQTPLT | FGGGTKVEIKR |
| | | | PGEPASISC | YLD | LLIY | | EAEDVGVYYC | | |
| | 66 | Germline | DIQMTQSPSSLSAS | RASQGIRNDLG | WYQQKPGKAPK | AASSLQS | GVPSRFSGSGSGTEFTLTISSL | LQHNSYPLT | FGGGTKVEIKR |
| | | | VGDRVTITC | | RLIY | | QPEDFATYYC | | |
| 1.29 | 4 | A30/JK4 | DIQMTQSPSSLSAS | RASQGIRNDLG | WYQQKPGKAPK | AASSLQS | GVPSRFSGSGSGTEFTLTISSL | LQHNSYPLT | FGGGTKVEIKR |
| | | | IGDRVTITC | | RLIY | | QPEDFATYYC | | |
| | 67 | Germline | DIVMTQTPLSSPVT | RSSQSLVHSDGNT | WLQQRPGQPPR | KISNRFS | GVPDRFSGSGAGTDFTLKISRV | MQATQFPXIT | FGQGTRLEIKR |
| | | | LGQPASISC | YLS | LLIY | | EAEDVGVYYC | | |
| 2.17 | 16 | A23/JK5 | EIQLTQSPLSSPVT | RSSQSLVHSDGDT | WLQQRPGQPPR | KISTRFS | GVPDRFSGSGAGTDFTLKISRV | MQTTQIPQIT | FGQGTRLEIKR |
| | | | LGQPASISC | YLN | LLIY | | ETDDVGIYYC | | |
| | 68 | Germline | DIQMTQSPSSLSAS | RASQSISSYLN | WYQQKPGKAPK | AASSLQS | GVPSRFSGSGSGTDFTLTISSL | QQSYSTPPT | FGQGTKVEIKR |
| | | | VGDRVTITC | | LLIY | | QPEDFATYYC | | |
| 2.24 | 20 | 012/JK1 | DIQLTQSPSSLSAS | RASQSIYSYLN | WYQQKPGKAPK | AASSLQS | GVPSRFSGSGSGTDFTLTISSL | QQSYSTPPT | FGQGTKVEIKR |
| | | | VGDRVTITC | | LLIY | | QPEDFATYYC | | |
| | 69 | Germline | DIVMTQTPLSSPVT | RSSQSLVHSDGNT | WLQQRPGQPPR | KISNRFS | GVPDRFSGSGAGTDFTLKISRV | MQATQFPQT | FGQGTKVEIKR |
| | | | LGQPASISC | YLS | LLIY | | EAEDVGVYYC | | |
| 1.37 | 8 | A23/JK1 | DIVMTQTPLSSTVI | RSSQSLVHSDGNT | WLQQRPGQPPR | MISNRFS | GVPDRFSGSGAGTDFTLKISRV | MQATESPQT | FGQGTKVEIKR |
| | | | LGQPASISC | YLN | LLIY | | EAEDVGVYYC | | |
| | 70 | Germline | DIVMTQTPLSLPVT | RSSQSLLDSDDGN | WYLQKPGQSPQ | TLSYRAS | GVPDRFSGSGSGTDFTLKISRV | MQRIEFPIT | FGQGTRLEIKR |
| | | | PGEPASISC | TYLD | LLIY | | EAEDVGVYYC | | |
| 2.70 | 44 | 01/JK5 | DIVMTQTPLSLPVT | RSSRSLLDSDDGN | WYLQKPGQSPQ | TLSYRAS | GVPDRFSGSGSGTDFTLKISRV | MQRVEFPIT | FGQGTRLEIKR |
| | | | PGEPASISC | TYLD | LLIY | | EAEDVGVYYC | | |
| 2.56 | 32 | | EIVMTQTPLSLPVT | RSSQSLLDSEDGN | WYLQKPGQSPQ | TLSHRAS | GVPDRFSGSGSGTDFTLKISRV | MQRVEFPIT | FGQGTRLEIKR |
| | | | PGEPASISC | TYLD | LLIY | | EAEDVGVYCC | | |
| 2.76 | 48 | | XXXXTQCPLSLPVT | RSSQSLLDSDDGN | WYLQKPGQSPQ | TVSYRAS | GVPDRFSGSGSGTDFTLKISRV | MQRIEFPIT | FGQGTRLEIKR |
| | | | PGEPASISC | TYLD | LLIY | | EAEDVGVYYC | | |
| | 71 | Germline | EIVLTQSPDFQSVT | RASQSIGSSLH | WYQQKPDQSPK | YASQSFS | GVPSRFSGSGSGTDFTLTINSL | HQSSSLPFT | FGPGTKVDIKR |
| | | | PKEKVTITC | | LLIK | | EAEDAATYYC | | |
| 2.59 | 36 | A26/JK3 | XXXXTQSPDFQSVT | RASQSIGSRLH | WYQQKPDQSPK | YASQSFS | GVPSRFSGSGSGTDFTLTINSL | HQSSNLPFT | FGPGTKVDIKR |
| | | | PKEKVTITC | | LLIK | | EAEDAATYYC | | |
| | 72 | Germline | DIQMTQSPSSLSAS | RASQGIRNDLG | WYQQKPGKAPK | AASSLQS | GVPSRFSGSGSGTEFTLTISSL | LQHNSYPXX | FGQGTKLEIKR |
| | | | VGDRVTITC | | RLIY | | QPEDFATYYC | | |
| 2.61 | 40 | A30/JK2 | DIQMTQSPSSRCAS | RASQGIRNDLA | WYQQKPGKAPK | AASSLQS | GVPSRFSGSRSGTEFTLTISSL | LQHNSYPPS | FGQGTKLEIKR |
| | | | VGDRVTITC | | RLIY | | QPEDFAAYYC | | |

Human antibody heavy chain VH3-33 was frequently selected in productive rearrangement for producing antibody successfully binding to TIM-1. Any variants of a human antibody VH3-33 germline in a productive rearrangement making antibody to TIM-1 is within the scope of the invention. Other heavy chain V regions selected in TIM-1 binding antibodies included: VH4-31, VH3-15, VH4-61, VH3-7 and VH3-48. The light chain V regions selected included: A27, A3, A30, A23, O12, O1, and A26. It is understood that the λκ XenoMouse® may be used to generate anti-TIM-1 antibodies utilizing lambda V regions.

The heavy chain variable domain germ line usage of the twelve anti-TIM-1 antibodies is shown in Table 6. The light chain variable domain germ line usage is shown in Table 7 (below).

TABLE 6
Germ Line Usage of the Heavy Chain Variable Domain Regions

| | V | V Se- | | | | D1 | | | | D2 Se- | | | | J Se- | Constant | | | |
|---|---|---|---|---|---|---|---|---|---|---|---|---|---|---|---|---|---|---|
| mAb | Heavy | quence | #N's | N | D1 | Sequence | #N's | N | D2 | quene | #N's | N | JH | quence | Region | CDR1 | CDR2 | CDR3 |
| 2.16 | VH3-15 | TGTACC | 5 | TCA | D3-16 | CGATAA | -N.A- | -N.A- | -N.A- | -N.A- | 7 | TGACGTG | JH4b | GACTAC | G4 | 64-93 | 136-192 | 289-309 |
| | (1-285) | | | GT | (291- | | | | | | | | (304- | | (344-529) | | | |
| | | | | | 296) | | | | | | | | 343) | | | | | |
| 2.70 | VH3-33 | GAGAGA | 0 | | D3-22 | TTACTATGAT | -N.A- | -N.A- | -N.A- | -N.A- | 15 | AGACATCA | JH4b | TTTGAC | G4 | 70-99 | 142-192 | 289-330 |
| | (1-290) | | | | (291- | AATAGT | | | | | | CTGGGGG | (322- | | (365-502) | | | |
| | | | | | 306) | (SEQ ID | | | | | | (SEQ ID | 364) | | | | | |
| | | | | | | NO: 73) | | | | | | NO: 74) | | | | | | |
| 2.59 | VH4-31 | GAGAGA | 8 | ATC | D6-13 | ATAGCAGCAA | -N.A- | -N.A- | -N.A- | -N.A- | 5 | TCGGG | JH4b | CTTTGA | G4 | 61-96 | 139-186 | 283-324 |
| | (2-284 | | | CCC | (293- | CTGGTAC | | | | | | | (315- | | (359-545) | | | |
| | | | | TC | 309) | (SEQ ID | | | | | | | 358) | | | | | |
| | | | | | | NO: 75) | | | | | | | | | | | | |
| 2.24 | VH3-33 | GAGAGA | 0 | | D3-22 | TTACTATGAT | -N.A- | -N.A- | -N.A- | -N.A- | 15 | AGACATCA | JH4b | TTTGAC | G4 | 76-105 | 148-198 | 295-336 |
| | (1-296) | | | | (297- | AATAGT | | | | | | CTGGGGG | (328- | | (371-568) | | | |
| | | | | | 312) | (SEQ ID | | | | | | (SEQ ID | 370) | | | | | |
| | | | | | | NO: 76) | | | | | | NO: 77) | | | | | | |
| 1.29 | VH4-61 | GAGAGA | 5 | TTA | D1-7 | ACTGGA | -N.A- | -N.A- | -N.A- | -N.A- | 6 | GCTTCC | JH4b | ACTTTG | G2 | 70-105 | 148-195 | 292-321 |
| | (1-293) | | | TG | (299- | | | | | | | | (311- | | (356-491) | | | |
| | | | | | 304) | | | | | | | | 355) | | | | | |
| 2.61 | VH3-33 | GAGAGA | 0 | | D3-22 | TTACTATGAT | -N.A- | -N.A- | -N.A- | -N.A- | 15 | AGACATCA | JH4b | TTTGAC | G4 | 76-105 | 148-198 | 295-336 |
| | (1-296) | | | | (297- | AATAGT | | | | | | CTGGGGG | (328- | | (371-534) | | | |
| | | | | | 312) | (SEQ ID | | | | | | (SEQ ID | 370) | | | | | |
| | | | | | | NO: 78) | | | | | | NO: 79) | | | | | | |
| 2.76 | VH3-33 | TGCGAG | 6 | GGA | D3-22 | CTATGATAGT | -N.A- | -N.A- | -N.A- | -N.A- | 7 | CGTTACC | JH6b | ACTACG | G4 | 64-93 | 136-186 | 283-324 |
| | (1-281) | | | TTT | (288- | AGT (SEQ | | | | | | | (308- | | (359-544) | | | |
| | | | | | 300) | ID NO: 80) | | | | | | | 358) | | | | | |
| 2.54 | VH3-33 | GCGAGA | -N.A- | -N.A- | -N.A- | -N.A- | -N.A- | -N.A- | -N.A- | -N.A- | 2 | TC | JH4b | TTGACT | G4 | 76-105 | 148-198 | 295-306 |
| | (1-296) | | | | | | | | | | | | (299- | | (341-537) | | | |

340)

| | | | | | | | | | | | | | | | | | | |
|---|---|---|---|---|---|---|---|---|---|---|---|---|---|---|---|---|---|---|
| 1.37 | VH3-7 | GCGAGA | -N.A- | -N.A- | -N.A- | -N.A- | -N.A- | -N.A- | -N.A- | -N.A- | 3 | TGG | JH4b | GACTAC | G2 | 82-111 | 154-204 | 301-309 |
| | (7-300) | | | | | | | | | | | | (304- | | (344-469) | | | |
| | | | | | | | | | | | | | 343) | | | | | |
| 2.17 | VH3-48 | TGTGCG | -N.A- | -N.A- | -N.A- | -N.A- | -N.A- | -N.A- | -N.A- | -N.A- | 5 | CGGGA | JH4b | CTTTGA | G4 | 76-105 | 148-198 | 295-306 |
| | (2-291) | | | | | | | | | | | | (297- | | (341-538) | | | |
| | | | | | | | | | | | | | 340) | | | | | |
| 2.45 | VH3-15 | CCACAG | 7 | TCG | D6-19 | CAGTGG | -N.A- | -N.A- | -N.A- | -N.A- | 0 | | JH4b | TGACTA | G4 | 61-90 | 133-189 | 286-306 |
| | (2-286) | | | ATA | (294- | | | | | | | | (300- | | (341-526) | | | |
| | | | | A | 299) | | | | | | | | 340) | | | | | |
| 2.56 | VH3-33 | GAGAGA | 0 | | D3-22 | TTACTATGAT | -N.A- | -N.A- | -N.A- | -N.A- | 20 | CGAGTCGG | JH4b | TTTGAC | G4 | 70-99 | 142-192 | 389-330 |
| | (1-290) | | | | (291- | A (SEQ ID | | | | | | CATCACTG | (322- | | (365-27) | | | |
| | | | | | 301) | NO: 81) | | | | | | GGGG | 364) | | | | | |

TABLE 7
Germ Line Usage of the Light Chain Variable Domain Regions

| | | V | | | | J | Constant | | | |
|---|---|---|---|---|---|---|---|---|---|---|
| mAb | VL | Sequence | #N's | N | JL | Sequence | Region | CDR1 | CDR2 | CDR3 |
| 2.70 | O1 (46-348) | TTTCCT | 0 | | JK5 | ATCACC | IGKC | 115-165 | 211-231 | 328-354 |
| | | | | | (349-385) | | (386-522) | | | |
| 2.59 | A26 (1-272) | TTTACC | 0 | | JK3 | ATTCAC | IGKC | 58-90 | 136-156 | 253-279 |
| | | | | | (273-310) | | (311-450) | | | |
| 2.24 | O12 (1-287) | CCCTCC | 0 | | JK1 | GACGTT | IGKC | 70-102 | 148-168 | 265-291 |
| | | | | | (288-322) | | (323-472) | | | |
| 1.29 | A30 (46-331) | ACCCTC | 0 | | JK4 | TCACTT | IGKC | 115-147 | 193-213 | 310-336 |
| | | | | | (332-367) | | (368-504) | | | |
| 2.56 | O1 (46-348) | TTTCCT | 0 | | JK5 | ATCACC | IGKC | 115-165 | 211-231 | 328-354 |
| | | | | | (349-385) | | (386-521) | | | |
| 2.61 | A30 (1-287) | CCCTCC | 3 | CAG | JK2 | TTTTGG | IGKC | 70-102 | 148-168 | 265-291 |
| | | | | | (291-322) | | (323-470) | | | |
| 2.76 | O1 (1-290) | GTTTCC | 0 | | JK5 | GATCAC | IGKC | 58-108 | 154-174 | 271-297 |
| | | | | | (291-328) | | (329-419) | | | |
| 1.37 | A23 (43-344) | TCCTCA | 0 | | JK1 | GACGTT | IGKC | 112-159 | 205-225 | 322-348 |
| | | | | | (345-379) | | (380-454) | | | |
| 2.17 | A23 (1-302) | TCCTCA | 1 | A | JK5 | ATCACC | IGKC | 70-117 | 163-183 | 280-309 |
| | | | | | (304-340) | | (341-490) | | | |
| 2.54 | A27 (1-286) | GCTCAC | 4 | TCCC | JK4 | GCTCAC | IGKC | 70-105 | 151-171 | 268-297 |
| | | | | | (291-328) | | (329-480) | | | |
| 2.16 | A3 (2-290) | AACTCC | 2 | GC | JK4 | TCACTT | IGKC | 61-108 | 154-174 | 271-297 |
| | | | | | (293-328) | | (329-447) | | | |
| 2.45 | A3 (1-287) | AACTCC | 2 | GC | JK4 | TCACTT | IGKC | 58-105 | 151-171 | 268-294 |
| | | | | | (290-325) | | (326-465) | | | |

The sequences encoding monoclonal antibodies 1.29, 1.37, 2.16, 2.17, 2.24, 2.45, 2.54 2.56, 2.59, 2.61, 2.70, and 2.76, respectively, including the heavy chain nucleotide sequence (A), heavy chain amino acid sequence (B) and the light chain nucleotide sequence (C) with the encoded amino acid sequence (D) are provided in the sequence listing as summarized in Table 1 above. A particular monoclonal antibody, 2.70, was further subcloned and is designated 2.70.2, see Table 1.

### Example 2

### Antibody Reactivity with Membrane Bound TIM-1 Protein by FACS

Fluorescent Activated Cell Sorter (FACS) analysis was performed to demonstrate the specificity of the anti-TIM-1 antibodies for cell membrane-bound TIM-1 antigen and to identify preferred antibodies for use as a therapeutic or diagnostic agent. The analysis was performed on two renal cancer cell lines, ACHN (ATCC#:CRL-1611) and CAKI-2 (ATCC#:HTB-47). A breast cancer cell line that does not express the TIM-1 antigen, BT549, was used as a control. Table 8 shows that both antibodies 2.59.2 and 2.70.2 specifically bound to TIM-1 antigen expressed on ACHN and CAKI-2 cells, but not antigen negative BT549 cells. Based on the Geo Mean Ratios normalized to the irrelevant antibody isotype control (pK16), ACHN cells had a higher cell surface expression of TIM-1 protein than CAKI-2 cells.

TABLE 8

| | | Geo Mean Ratio (relative to negative control) | | |
|---|---|---|---|---|
| Antibody | BIN | ACHN | CAKI-2 | BT549 |
| 2.59.2 | 1 | 15.2 | 7.7 | 1.4 |
| 2.70.2 | 6 | 19.4 | 8.8 | 1.8 |
| 1.29 | 1 | 17.9 | | 1.2 |
| 2.16.1 | 2 | 7.9 | | 1.5 |
| 2.56.2 | 5 | 12.2 | | 1.5 |
| 2.45.1 | 8 | 4.3 | | 1.1 |

### Example 3

### Specificity of the Anti-TIM-1 Monoclonal Antibodies

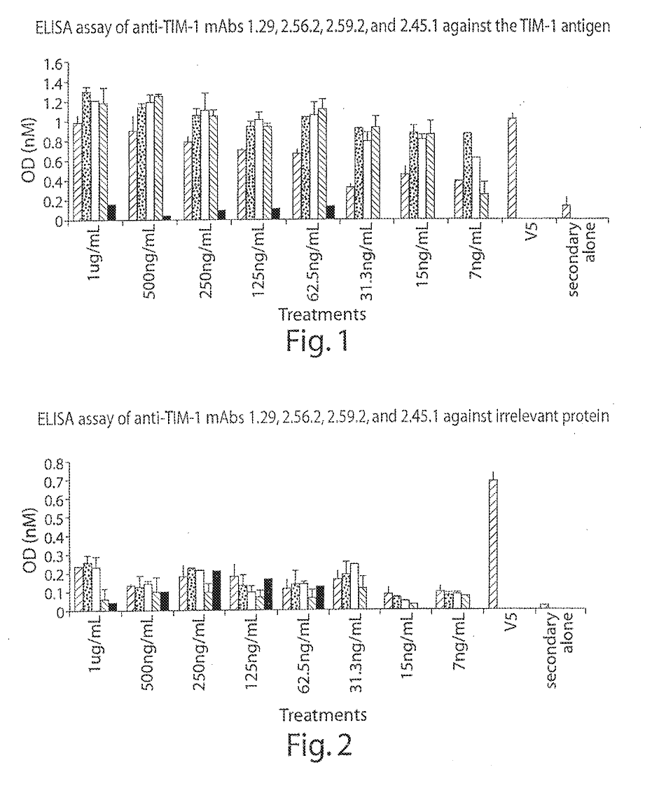

The anti-TIM-1 antibodies bound specifically to TIM-1 protein but not an irrelevant protein in an ELISA assay. TIM-1 antigen (with a V5-HIS tag) specific binding results for four of the anti-TIM-1 monoclonal antibodies (1.29, 2.56.2, 2.59.2, and 2.45.1) as well as an isotype matched control mAb PK16.3 are shown in FIG. 1. The X axis depicts the antibodies used in the order listed above and the Y axis is the optical density. The respective binding of these antibodies to the irrelevant protein (also with a V5-HIS tag) is shown in FIG. 2.

### ELISA Protocol.

A 96-well high protein binding ELISA plate (Corning Costar cat. no. 3590) was coated with 50 μL of the TIM-1 antigen at a concentration of 5 μg/mL diluted in coating buffer (0.1M Carbonate, pH9.5), and incubated overnight at 4° C. The wells were then washed five times with 200-300 μL of 0.5% Tween-20 in PBS. Next, plates were blocked with 200 μL of assay diluent (Pharmingen, San Diego, Calif., cat. no. 26411E) for at least 1 hour at room temperature. Anti-TIM-1 monoclonal antibodies were then diluted in assay diluent with the final concentrations of 7, 15, 31.3, 62.5, 125, 250, 500 and 1000 ng/mL. An anti-V5-HRP antibody was used at 1:1000 to detect the V5 containing peptide as the positive control for the ELISA. Plates were then washed again as described above. Next 50 μL of each antibody dilution was added to the proper wells, then incubated for at least 2 hours at room temp. Plates were washed again as described above, then 50 μL of secondary antibody (goat anti-human-HRP) was added at 1:1000 and allowed to incubate for 1 hour at room temp. Plates were washed again as described above then developed with 100 μL of TMB substrate solution/well (1:1 ratio of solution A+B) (Pharmingen, San Diego, Calif., cat. no. 2642KK). Finally, the reaction was stopped with 50 μL sulfuric acid and the plates read at 450 nm with a correction of 550 nm.

### Example 4

### Antibody Sequences

In order to analyze structures of antibodies, as described herein, genes encoding the heavy and light chain fragments out of the particular hybridoma were cloned. Gene cloning and sequencing was accomplished as follows. Poly(A)+mRNA was isolated from approximately 2×105 hybridoma cells derived from immunized XenoMouse® mice using a Fast-Track kit (Invitrogen). The generation of random primed cDNA was followed by PCR. Human VH or human Vκ family specific variable domain primers (Marks et. al., 1991) or a universal human VH primer, MG-30 (CAGGTGCAGCTGGAGCAGTCIGG) (SEQ ID NO:83) were used in conjunction with primers specific for the human:

Cγ2 constant region
(MG-40d; 5'-GCT GAG GGA GTA GAG TCC TGA GGA-3'
(SEQ ID NO: 84));
Cγ1 constant region
(HG1; 5' CAC ACC GCG GTC ACA TGG C (SEQ ID NO: 85));
or
Cγ3 constant region
(HG3; 5' CTA CTC TAG GGC ACC TGT CC (SEQ ID NO: 86))

or the human Cκ constant domain (hκP2; as previously described in Green et al., 1994). Sequences of human MAbs-derived heavy and kappa chain transcripts from hybridomas were obtained by direct sequencing of PCR products generated from poly(A⁺) RNA using the primers described above. PCR products were also cloned into pCRII using a TA cloning kit (Invitrogen) and both strands were sequenced using Prism dye-terminator sequencing kits and an ABI 377 sequencing machine. All sequences were analyzed by alignments to the “V BASE sequence directory” (Tomlinson et al., MRC Centre for Protein Engineering, Cambridge, UK) using MacVector and Geneworks software programs.

In each of Tables 4-7 above, CDR domains were determined in accordance with the Kabat numbering system. See Kabat, Sequences of Proteins of Immunological Interest (National Institutes of Health, Bethesda, Md. (1987 and 1991)).

### Example 5

### Epitope Binning and BiaCore® Affinity Determination

### Epitope Binning

Certain antibodies, described herein were “binned” in accordance with the protocol described in U.S. Patent Application Publication No. 20030157730, published on Aug. 21, 2003, entitled “Antibody Categorization Based on Binding Characteristics.”

MxhIgG conjugated beads were prepared for coupling to primary antibody. The volume of supernatant needed was calculated using the following formula: (n+10)×50 μL (where n=total number of samples on plate). Where the concentration was known, 0.5 μg/mL was used. Bead stock was gently vortexed, then diluted in supernatant to a concentration of 2500 of each bead per well or 0.5×105/mL and incubated on a shaker in the dark at room temperature overnight, or 2 hours if at a known concentration of 0.5 μg/mL. Following aspiration, 50 μL of each bead was added to each well of a filter plate, then washed once by adding 100 μL/well wash buffer and aspirating. Antigen and controls were added to the filter plate 50 μL/well then covered and allowed to incubate in the dark for 1 hour on shaker. Following a wash step, a secondary unknown antibody was added at 50 μL/well using the same dilution (or concentration if known) as used for the primary antibody. The plates were then incubated in the dark for 2 hours at room temperature on shaker followed by a wash step. Next, 50 μL/well biotinylated mxhIgG diluted 1:500 was added and allowed to incubate in the dark for 1 hour on shaker at room temperature. Following a wash step, 50 μL/well Streptavidin-PE was added at 1:1000 and allowed to incubate in the dark for 15 minutes on shaker at room temperature. Following a wash step, each well was resuspended in 80 μL blocking buffer and read using a Luminex system.

Table 9 shows that the monoclonal antibodies generated belong to eight distinct bins. Antibodies bound to at least three distinct epitopes on the TIM-1 antigen.

### Determination of Anti-TIM-1 mAb Affinity Using BiaCore® Analysis

BiaCore® analysis was used to determine binding affinity of anti-TIM-1 antibody to TIM-1 antigen. The analysis was performed at 25° C. using a BiaCore® 2000 biosensor equipped with a research-grade CMS sensor chip. A high-density goat a human antibody surface over a CMS BiaCore® chip was prepared using routine amine coupling. Antibody supernatants were diluted to ˜5 μg/mL in HBS-P running buffer containing 100 μg/mL BSA and 10 mg/mL carboxymethyldextran. The antibodies were then captured individually on a separate surface using a 2 minute contact time, and a 5 minute wash for stabilization of antibody baseline.

TIM-1 antigen was injected at 292 nM over each surface for 75 seconds, followed by a 3-minute dissociation. Double-referenced binding data were obtained by subtracting the signal from a control flow cell and subtracting the baseline drift of a buffer inject just prior to the TIM-1 injection. TIM-1 binding data for each mAb were normalized for the amount of mAb captured on each surface. The normalized, drift-corrected responses were also measured. The kinetic analysis results of anti-TIM-1 mAB binding at 25° C. are listed in Table 9 below.

TABLE 9
Competition Bins and KDs for TIM-1-specific mAbs

| | | Affinity nM |
|---|---|---|
| Bin | Antibody | by BIAcore |
| 1 | 2.59 | 0.38 |
| | 1.29 | 3.64 |
| 2 | 2.16 | 0.79 |
| 3 | 2.17 | 2.42 |
| 4 | 1.37 | 2.78 |
| | 2.76 | 0.57 |
| | 2.61 | 1.0 |
| 5 | 2.24 | 2.42 |
| | 2.56 | 1.1 |
| 6 | 2.70 | 2.71 |
| 7 | 2.54 | 3.35 |
| 8 | 2.45 | 1.15 |

### Example 6

### Epitope Mapping

Anti-TIM-1 mAb 2.70.2 was assayed for reactivity against overlapping peptides designed from the TIM-1 antigen sequence. Assay plates were coated with the TIM-1 fragment peptides, using irrelevant peptide or no peptide as controls. Anti-TIM-1 mAb 2.70.2 was added to the plates, incubated, washed and then bound antibody was detected using anti-human Ig HRP conjugate. Human antibody not specific to TIM-1, an isotype control antibody or no antibody served as controls. Results showed that mAb 2.70.2 specifically reacted with a peptide having the amino acid sequence PMPLPRQNHEPVAT (SEQ ID NO:87), corresponding to amino acids 189-202 of the TIM-1 immunogen (SEQ ID NO:50).

Specificity of mAb 2.70.2 was further defined by assaying against the following peptides:

A)
(SEQ ID NO: 87)
PMPLPRQNHEPVAT
B)
(SEQ ID NO: 88)
PMPLPRQNHEPV
C)
(SEQ ID NO: 89)
PMPLPRQNHE
D)
(SEQ ID NO: 90)
PMPLPRQN
E)
(SEQ ID NO: 91)
PMPLPR
F)
(SEQ ID NO: 92)
PLPRQNHEPVAT
G)
(SEQ ID NO: 93)
PRQNHEPVAT
H)
(SEQ ID NO: 94)
QNHEPVAT
I)
(SEQ ID NO: 95)
HEPVAT

Results showed mAb 2.70.2 specifically bound to peptides A, B, C, and F, narrowing the antibody epitope to PLPRNHE (SEQ ID NO:96)

As shown in Table 10, synthetic peptides were made in which each amino acid residue of the epitope was replace with an alanine and were assayed for reactivity with mAb 2.70.2. In this experiment, the third proline and the asparagines residues were determined to be critical for mAb 2.70.2 binding. Furthermore, assays of peptides with additional N or C terminal residues removed showed mAb 2.70.2 binding was retained by the minimal epitope

| | |
|---|---|
| LPRQNH | (SEQ ID NO: 97) |

TABLE 10

| | | | | | | | | | | SEQ ID | mAb2.70.2 |
|---|---|---|---|---|---|---|---|---|---|---|---|
| | | | | | | | | | | NO: | Reactivity |
| P | M | P | L | P | R | Q | N | H | E | 89 | + |
| P | M | P | A | P | R | Q | N | H | E | 98 | + |
| P | M | P | L | A | R | Q | N | H | E | 99 | − |
| P | M | P | L | P | A | Q | N | H | E | 100 | + |
| P | M | P | L | P | R | A | N | H | E | 101 | + |
| P | M | P | L | P | R | Q | A | H | E | 102 | − |
| P | M | P | L | P | R | Q | N | A | E | 103 | + |
| | | P | L | P | R | Q | N | H | E | 104 | + |
| | | | L | P | R | Q | N | H | E | 105 | + |
| | | P | L | P | R | Q | N | H | E | 106 | + |
| | | | L | P | R | Q | N | H | E | 107 | + |

### Example 7

### Immunohistochemical (IHC) Analysis of TIM-1 Expression in Normal and Tumor Tissues

Immunohistochemical (IHC) analysis of TIM-1 expression in normal and tumor tissue specimens was performed with techniques known in the art. Biotinylated fully human anti-TIM-1 antibodies 2.59.2, 2.16.1 and 2.45.1 were analyzed. Streptavidin-HRP was used for detection.

Briefly, tissues were deparaffinized using conventional techniques, and then processed using a heat-induced epitope retrieval process to reveal antigenic epitopes within the tissue sample. Sections were incubated with 10% normal goat serum for 10 minutes. Normal goat serum solution was drained and wiped to remove excess solution. Sections were incubated with the biotinylated anti-TIM-1 mAb at 5 μg/mL for 30 minutes at 25° C., and washed thoroughly with PBS. After incubation with streptavidin-HRP conjugate for 10 minutes, a solution of diaminobenzidine (DAB) was applied onto the sections to visualize the immunoreactivity. For the isotype control, sections were incubated with a biotinylated isotype matched negative control mAb at 5 μg/mL for 30 minutes at 25° C. instead of biotinylated anti-TIM-1 mAb. The results of the IHC studies are summarized in Tables 11 and 12.

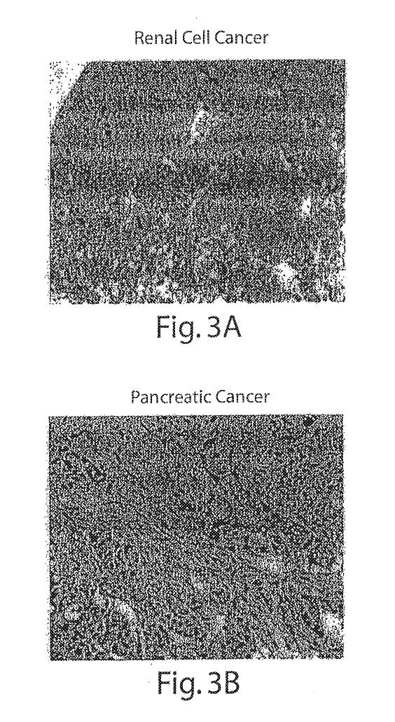

The specimens were graded on a scale of 0-3, with a score of 1+ indicating that the staining is above that observed in control tissues stained with an isotype control irrelevant antibody. The corresponding histological specimens from one renal tumor and the pancreatic tumor are shown in FIG. 3A and FIG. 3B. In addition to these the renal and pancreatic tumors, specimens from head and neck cancer, ovarian cancer, gastric cancer, melanoma, lymphoma, prostate cancer, liver cancer, breast cancer, lung cancer, bladder cancer, colon cancer, esophageal cancer, and brain cancer, as well the corresponding normal tissues were stained with anti-TIM-1 mAb 2.59.2. Overall, renal cancer tissue samples and pancreatic cancer tissue samples highly positive when stained with anti-TIM-1 mAb 2.59.2. No staining in normal tissues was seen. These results indicate that TIM-1 is a marker of cancer in these tissues and that anti-TIM-1 mAb can be used to differentiate cancers from normal tissues and to target TIM-1 expressing cells in vivo.

TABLE 11
Immunohistology Renal tumors expression of TIM-1
protein detected by anti-TIM-1 mAb 2.59.2

| Specimen | Cell Type | Histology | Score |
|---|---|---|---|
| 1 | Malignant cells | Not known | 0 |
| 1 | Other | Not cell associated | 2 |
| 2 | Malignant cells | Clear Cell | 2 |
| 3 | Malignant cells | Clear Cell | 0 |
| 4 | Malignant cells | Clear Cell | 3 |
| 5 | Malignant cells | Clear Cell | 2 (occasional) |
| 6 | Malignant cells | Not known | 2 |
| 7 | Malignant cells | Clear Cell | 2 |
| 8 | Malignant cells | Clear Cell | 0 |
| 9 | Malignant cells | Clear Cell | 2 (occasional) |
| 10 | Malignant cells | Clear Cell | 1-2 |
| 11 | Malignant cells | Not known | 3 (many) |
| 12 | Malignant cells | Clear Cell | 1-2 |
| 12 | Other | Not cell associated | 2 |
| 13 | Malignant cells | Clear Cell | 2 (occasional) |
| 14 | Malignant cells | Clear Cell | 1-2 |
| 15 | Malignant cells | Clear Cell | 3-4 |
| 16 | Malignant cells | Not known | 1-2 |
| 17 | Malignant cells | Not known | 4 (occasional) |
| 18 | Malignant cells | Not known | 1-2 |
| 19 | Malignant cells | Clear Cell | 0 |
| 20 | Malignant cells | Clear Cell | 3-4 |
| 21 | Malignant cells | Clear Cell | 2 (occasional) |
| 22 | Malignant cells | Clear Cell | 3 |
| 23 | Malignant cells | Clear Cell | 2 |
| 24 | Malignant cells | Not known | 3-4 occasional |
| 25 | Malignant cells | Not known | 2-3 |
| 26 | Malignant cells | Not known | 3 |
| 27 | Malignant cells | Clear Cell | 2 |
| 27 | Other | Not cell associated | 2 |
| 28 | Malignant cells | Not known | 2 |
| 29 | Malignant cells | Clear Cell | 2-3 |
| 30 | Malignant cells | Clear Cell | 2 |
| 31 | Malignant cells | Clear Cell | 2-3 |
| 32 | Malignant cells | Clear Cell | 0 |
| 33 | Malignant cells | Clear Cell | 0 |
| 34 | Malignant cells | Clear Cell | 2 |
| 34 | Other | Not cell associated | 2 |
| 35 | Malignant cells | Clear Cell | 2-3 |
| 36 | Malignant cells | Clear Cell | 3 |
| 37 | Malignant cells | Not known | 3 |
| 38 | Malignant cells | Clear Cell | 3 |
| 39 | Malignant cells | Not known | 2 |
| 40 | Malignant cells | Clear Cell | 2-3 |

TABLE 12
Normal Human Tissue Immunohistology
with anti-TIM-1 mAb 2.59.2

| | Score | | |
|---|---|---|---|
| Tissue | Specimen 1 | | Specimen 2 |
| Adrenal Cortex | 0 | | 0 |
| Adrenal Medulla | 0 | | 1 |
| Bladder: Smooth muscle | 0 | | 0 |
| Bladder: Transitional Epithelium | 3 | | 0 |
| Brain cortex: Blia | 0 | | 0 |
| Brain cortex: Neurons | 0 | | 0 |
| Breast: Epithelium | 0 | | 0 |
| Breast: Stroma | 0 | | 0 |
| Colon: Epithelium | 0 | | 0 |
| Colon: Ganglia | 0 | | NA |
| Colon: Inflammatory compartment | 3-4 | (occasional) | 3 (occasional) |
| Colon: Smooth muscle | 1 | (occasional) | 0 |
| Heart: Cardiac myocytes | 0 | | 0 |
| Kidney cortex: Glomeruli | 2-3 | | 2 |
| Kidney cortex: Tubular epithelium | 2 | | 2-3 |
| Kidney medulla: Tubular | 2 | | 0 |
| epithelium | | | |
| Kidney medulla: other | NA | | 2-3 |
| Liver: Bile duct epithelium | 0 | | 0 |
| Liver: Hepatocytes | 1-2 | | 1 |
| Liver: Kupffer cells | 0 | | 0 |
| Lung: Airway epithelium | 0 | | 0 |
| Lung: Alveolar macrophages | 2 (occasional)-3 | | 2-3 (occasional) |
| Lung: other | 3 | | NA |
| Lung: Pneumocytes | 2-3 | (occasional) | 2-3 (occasional) |
| Ovary: Follicle | 2 | (occasional) | 1-2 |
| Ovary: Stroma | 1 | | 1 (occasional) |
| Pancreas: Acinar epithelium | 0 | | 1 (occasional) |
| Pancreas: Ductal epithelium | 0 | | 0 |
| Pancreas: Islets of Langerhans | 0 | | 0 |
| Placenta: Stroma | 0 | | 0 |
| Placenta: Trophoblasts | 0 | | 0 |
| Prostate: Fibromuscular stroma | 0 | | 0 |
| Prostate: Glandular epithelium | 0 | | 0 |
| Skeletal muscle: Myocytes | 0 | | 0 |
| Skin: Dermis | 0 | | 0 |
| Skin: Epidermis | 0 | | 0 |
| Small intestine: Epithelium | 0 | | 0 |
| Small intestine: Ganglion | 0 | | 0 |
| Small intestine: Inflammatory | 0 | | 0 |
| compartment | | | |
| Small intestine: Smooth muscle | 0 | | 0 |
| cells | | | |
| Spleen: Red pulp | 0 | | 2 (rare) |
| Spleen: white pulp | 0 | | 0 |
| Stomach: Epithelium | 0 | | 0 |
| Stomach: Smooth Muscle Cells | 0 | | 0 |
| Tstis: Leydig cells | 2 | | 1-2 |
| Testis: Seminiferous epithelium | 1 | | 2 |
| Thymus: Epithelium | 0 | | 0 |
| Thymus: Lymphocytes | 2 | (rare) | 2 (occasional) |
| Thyroid: Follicular epithelium | 0 | | 0 |
| Tonsil: Epithelium | 0 | | 0 |
| Tonsil: Lymphocytes | 3 | (occasional) | 2 (occasional) |

| | | |
|---|---|---|
| Uterus: Endometrium | 0 | 0 |
| Uterus: Myometrium | 0 | 0 |

### Example 8

### Antibody Mediated Toxin Killing

A clonogenic assay as described in the art was used to determine whether primary antibodies can induce cancer cell death when used in combination with a saporin toxin conjugated secondary antibody reagent. Kohls and Lappi, Biotechniques, 28(1):162-5 (2000).

### Assay Protocol

ACHN and BT549 cells were plated onto flat bottom tissue culture plates at a density of 3000 cells per well. On day 2 or when cells reached ˜25% confluency, 100 ng/well secondary mAb-toxin (goat anti-human IgG-saporin; Advanced Targeting Systems; HUM-ZAP; cat. no. IT-22) was added. A positive control anti-EGFR antibody, mAb 2.7.2, mAb 2.59.2, or an isotype control mAb was then added to each well at the desired concentration (typically 1 to 500 ng/mL). On day 5, the cells were trypsinized, transferred to a 150 mm tissue culture dish, and incubated at 37° C. Plates were examined daily. On days 10-12, all plates were Giemsa stained and colonies on the plates were counted. Plating efficiency was determined by comparing the number of cells prior to transfer to 150 mm plates to the number of colonies that eventually formed.

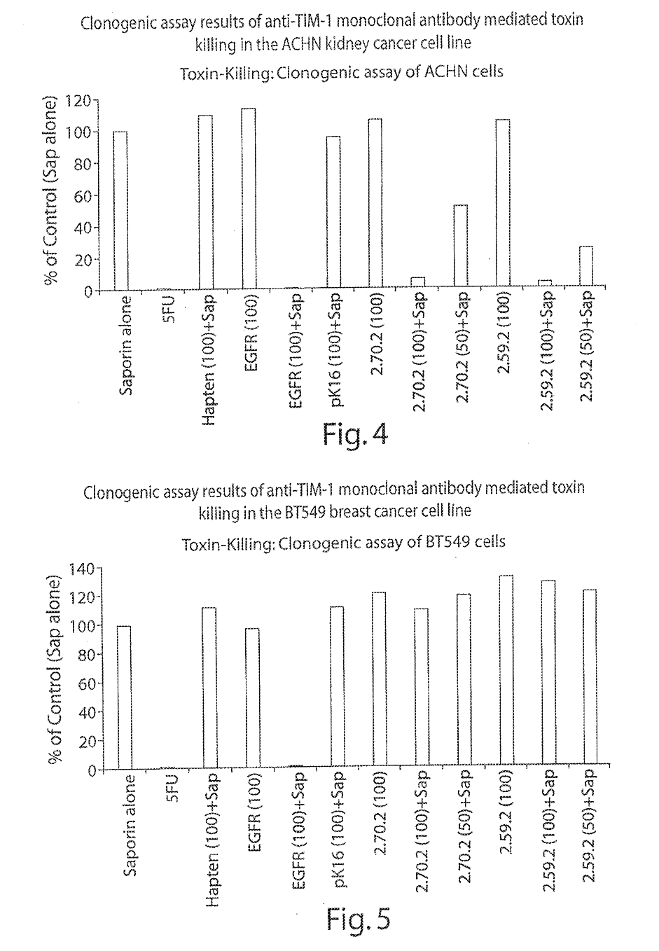

The percent viability in antigen positive ACHN and antigen negative BT549 cell lines are presented in FIG. 4 and FIG. 5 respectively. In this study, the cytotoxic chemotherapy reagent 5 Fluorouracil (5-FU) was used as the positive control and induced almost complete killing, whereas the saporin conjugated-goat anti-human secondary antibody alone had no effect. A monoclonal antibody (NeoMarkers MS-269-PABX) generated against the EGF receptor expressed by both cell lines was used to demonstrate primary antibody and secondary antibody-saporin conjugate specific killing. The results indicate that both cell lines were susceptible to EGFR mAb mediated toxin killing at 100 ng/mL. At the same dose, both the anti-TIM-1 mAb 2.59.2 and the anti-TIM-1 mAb 2.70.2 induced over 90% ACHN cell death as compared to 0% BT549 cell death.

### Antibody Toxin Conjugate Mediated Killing: Clonogenic Assay

CAKI-1 and BT549 cells were plated onto flat bottom tissue culture plates at a density of 3000 cells per well. On day 2 or when cells reach ˜25% confluency, various concentrations (typically 1 to 1000 ng/ml) of unconjugated and Auristatin E (AE)-conjugated mAb, which included anti-EGFR, anti-TIM-1 mAb 2.7.2, anti-TIM-1 mAb 2.59.2 or isotype control mAb, were added to cells. Each of these antibodies was conjugated to AE. The monoclonal antibody (NeoMarkers MS-269-PABX) generated against the EGF receptor, which is expressed by both cell lines, was used as a positive control to demonstrate specific killing mediated by AE-conjugated antibody. On day 5, the cells were trypsinized, transferred to a 150 mm tissue culture dish, and incubated at 37° C. Plates were examined daily. On days 10-12, all plates were Giemsa stained and colonies on the plates were counted. Plating efficiency was determined by counting the cells prior to transfer to 150 mm plates and compared to the number of colonies that eventually formed.

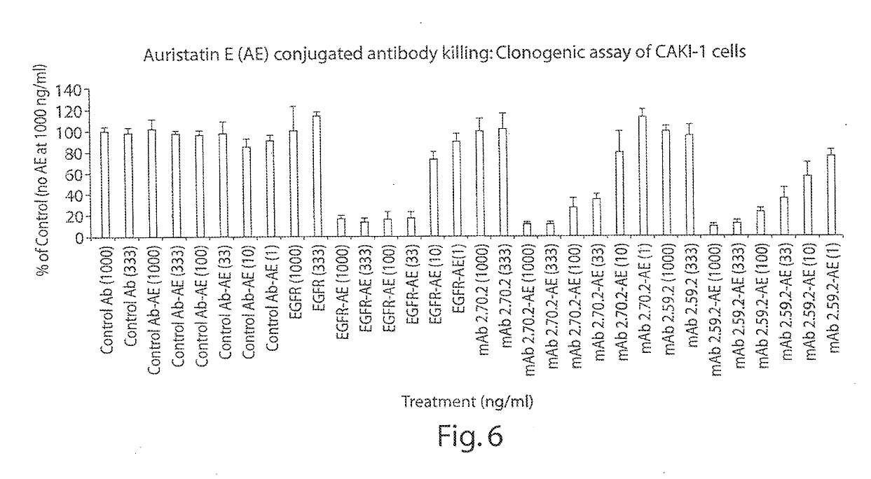

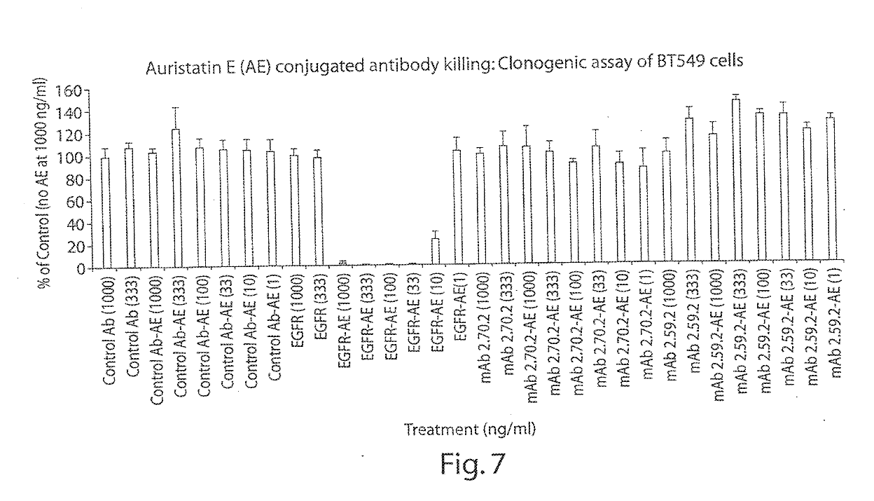

The percent viability in antigen positive CAKI-1 and antigen negative BT549 cell lines are presented in FIGS. 6 and 7, respectively.

The results indicate that unconjugated and AE-conjugated isotype control mAb had no effect on growth of both CAKI-1 and BT549 cells. However, both cell lines were susceptible to AE-EGFR mAb mediated toxin killing in a dose-dependent fashion. At the maximum dose, both anti-TIM-1 mAbs (2.59.2 and 2.70.2) induced over 90% CAKI-1 cell death when compared to their unconjugated counterparts. The response was dose dependent. At the same dose range, both anti-TIM-1 mAbs 2.59.2 and 2.70.2 did not affect the survival of BT549 cells.

### Example 9

### Human Tumor Xenograft Growth Delay Assay

A tumor growth inhibition model was used according to standard testing methods. Geran et al., Cancer Chemother. Rep. 3:1-104 (1972). Athymic nude mice (nu/nu) were implanted with either tumor cells or tumor fragments from an existing host, in particular, renal (CaKi-1) or ovarian (OVCAR) carcinoma tumor fragments were used. These animals were then treated with an anti-TIM-1 antibody immunotoxin conjugate, for example, mAb 2.70.2 AE conjugate at doses ranging from 1 to 20 mg/kg body weight, twice weekly for a period of 2 weeks. Tumor volume for treated animals was assessed and compared to untreated control tumors, thus determining the tumor growth delay.

After reaching a volume of 100 mm3 animals are randomized and individually identified in groups of 5 individuals per cage. Protein or antibody of interest was administered via conventional routes (intraperitoneal, subcutaneous, intravenous, or intramuscular) for a period of 2 weeks. Twice weekly, the animals are evaluated for tumor size using calipers. Daily individual animal weights are recorded throughout the dosing period and twice weekly thereafter. Tumor volume is determined using the formula: Tumor volume (in mm3)=(length×width×height)×0.536. The volume determinations for the treated groups are compared to the untreated tumor bearing control group. The difference in time for the treated tumors to reach specific volumes is calculated for 500 1000, 1500 and 2000 mm3. Body weights are evaluated for changes when compared to untreated tumor bearing control animals. Data are reported as tumor growth in volume plotted against time. Body weights for each experimental group are also plotted in graph form.

Results show that the treatment is well tolerated by the mice. Specifically, complete regressions were noted in both the IGROV1 ovarian (6.25 mg/kg i.v. q4dX4) and the Caki-1 (3.3 mg/kg i.v. q4dx4) renal cell carcinoma models. No overt toxicity was observed in mice at doses up to 25 mg/kg (cumulative dose of 100 mg/kg). These data indicate that treatment with anti-TIM-1 mAb AE conjugate inhibits tumor growth of established CaKi-1 and OVCAR tumors, thus making these antibodies useful in the treatment of ovarian and renal carcinomas.

### Example 10

### Treatment of Renal Carcinoma with Anti-TIM-1 Antibodies

A patient in need of treatment for a renal carcinoma is given an intravenous injection of anti-TIM-1 antibodies coupled to a cytotoxic chemotherapic agent or radiotherapic agent. The progress of the patient is monitored and additional administrations of anti-TIM-1 antibodies are given as needed to inhibit growth of the renal carcinoma. Following such treatment, the level of carcinoma in the patient is decreased.

### Example 11

### FACS Analysis of Expression of TIM-1 Protein on CD4+ T Cells

Mononuclear cells were isolated from human blood diluted 1:1 in PBS, by spinning over Ficoll for 20 minutes. The mononuclear cells were washed twice at 1000 rpm with PBS-Mg and Ca and re-suspended in Miltenyi buffer (Miltenyi Biotec Inc., Auburn, Calif.); PBS, 0.5% BSA, 5 mM EDTA at approximately 108 cells/mL. 20 μL of CD4 Miltenyi beads were added per 107 cells and incubated for 15 minutes on ice. Cells were washed with a 10-fold excess volume of Miltenyi buffer. A positive selection column (type VS+) (Miltenyi Biotec Inc., Auburn, Calif.) was washed with 3 mL of Miltenyi buffer. The pelleted cells were re-suspended at 108 cells per mL of Miltenyi buffer and applied to the washed VS column. The column was then washed three times with 3 mL of Miltenyi buffer. Following this, the VS column was removed from the magnetic field and CD4+ cells were eluted from the column with 5 mL of Miltenyi buffer. Isolated CD4+ lymphocytes were pelleted and re-suspended in DMEM 5% FCS plus additives (non-essential amino acids, sodium pyruvate, mercaptoethanol, glutamine, penicillin, and streptomycin) at 106 cells/mL. 1×106 freshly isolated resting CD4+ T cells were transferred into flow cytometry tubes and washed with 2 mL/tube FACS staining buffer (FSB) containing PBS, 1% BSA and 0.05% NaN3. Cells were spun down and supernatant removed. Cells were blocked with 20% goat serum in FSB for 30 minutes on ice. Cells were washed as above and incubated with 10 μg/mL of primary human anti-TIM-1 mAb or control PK16.3 mAb in FSB (200 μL) for 45 minutes on ice followed by washing. Secondary goat anti-human PE conjugated antibody was added at 1:50 dilution for 45 minutes on ice in the dark, washed, resuspended in 500 μL of PBS containing 1% formaldehyde and kept at 4° C. until flow cytometry analysis was performed.

FACS analysis was performed to determine the expression of TIM-1 protein as detected with five anti-TIM-1 monoclonal antibodies (2.59.2, 1.29, 2.70.2, 2.56.2, 2.45.1) on human and mouse resting CD4+ T cells, as well as human activated and human polarized CD4+ T cells. These analyses demonstrate that freshly isolated resting human CD4+ T cells do not express TIM-1, while a major fraction of polarized human Th2 and Th1 cells do express TIM-1.

FACS Analysis of the Expression of the TIM-1 protein on human CD4+Th2 cells using five anti-TIM-1 monoclonal antibodies is shown in Table 13. The experiment is described in the left-hand column and the labeled antibody is specified along the top row. Data is reported as the geometric mean of the fluorescence intensity.

TABLE 13
FACS Analysis of the Expression ofthe TIM-1 protein on
human CD4+ Th2 cells

| | Geometric mean of fluorescence intensity | | | | | |
|---|---|---|---|---|---|---|
| | Control | Anti-TIM-1 mAb | | | | |
| Experiment | PK16.3 | 1.29 | 2.45.1 | 2.56.2 | 2.59.2 | 2.70.2 |
| Resting Human | 4.6 | 4.7 | 5.1 | 6 | 4.9 | N/A |
| CD4+ T cells | | | | | | |
| Polarized | 8.4 | 22.3 | 42.4 | 564.1 | 22 | 27.8 |
| Human CD4+ | | | | | | |
| Th2 Cells | | | | | | |

Table 14 demonstrates that over the course of 5 days, continual stimulation of T cells results in an increase in TIM-1 expression, as measured by anti-TIM-1 mAb 2.70.2, as compared to the control PK16.3 antibody. Furthermore, addition of matrix metalloproteinase inhibitor (MMPI) did not measurably increase TIM-1 expression, demonstrating that the receptor is not shed from T cells under these experimental conditions. Thus, expression of the TIM-1 protein and specific antibody binding is specific to activated Th1 and Th2 cells, which in turn, are characteristic of inflammatory response, specifically asthma.

TABLE 14
Percent of activated T cells that express TIM-1

| | | Day 0 | Day 1 | Day 2 | Day 4 | Day 5 |
|---|---|---|---|---|---|---|
| Control | −MMPI | 1 | 3 | 3 | 1 | 1 |
| PK16.3 | +MMPI | 1 | 2 | 6 | 2 | 2 |
| TIM-1 | −MMPI | 1 | 8 | 10 | 5 | 13 |
| 2.70.2 | +MMPI | 1 | 10 | 14 | 10 | 19 |

### Example 12

### Cytokine Assays

IL-4, IL-5, IL-10, IL-13, and IFNγ production levels by activated Th1 and Th2 cell were measured in culture supernatants treated with anti-TIM-1 antibodies using standard ELISA protocols. Cytokine production by Th1 or Th2 cells treated with anti-TIM-1 antibodies was compared to Th1 or Th2 cells treated with the control PK16.3 antibody. In addition, the following samples were run in parallel as internal controls: i) anti-CD3 treated Th1 or Th2 cells, where no cytokine production is expected because of the absence of co-stimulation, ii) anti-CD3/anti-CD28 stimulated Th1 or Th2 cells, expected to show detectable cytokine production, and iii) untreated Th1 or Th2 cells. CD4+ T cells were isolated as described in the Example above. Isolated CD4+ lymphocytes were then spun down and re-suspended in DMEM 5% FCS plus additives (non-essential amino acids, sodium pyruvate, mercaptoethanol, glutamine, penicillin, and streptomycin) at 10⁶ cells/mL. Falcon 6-well non-tissue culture treated plates were pre-coated overnight with anti-CD3 (2 μg/mL) and anti-CD28 (10 μg/mL) (600 μL total in Dulbecco's PBS) overnight at 4° C. The plates were washed with PBS and CD4+ lymphocytes were suspended at 500,000 cells/mL in Th2 medium: DMEM+10% FCS plus supplements and IL-2 5 ng/mL, IL-4 5 ng/mL, anti-IFN gamma 5 μg/mL and cells were stimulated 4-6 days at 37° C. and 5% CO2 in the presence of 5 μg/mL of mAb recognizing the TIM-1 protein or isotype matched negative control mAb PK16.3.

In another set of experiments, CD4+ lymphocytes were suspended at 500,000 cells/mL in Th1 medium: DMEM+10% FCS plus supplements and IL-2 5 ng/mL, IL12 5 ng/mL, anti-IL-4 5 μg/mL and stimulated 4-6 days 37° C. temp and 5% CO2 in the presence of 5 μg/mL TIM-1 or isotype matched control mAb PK16.3. Cells were washed two times in DMEM and resuspended in DMEM, 10% FCS plus supplements and 2 ng/mL IL-2 (500,000 cells/mL) in the presence of 5 μg/mL TIM-1 mAb or control PK16.3 mAb and cultured (rested) for 4-6 days at 37° C. and 5% CO2. The process of activation and resting was repeated at least once more as described above with the addition of anti-CD95L (anti-FAS ligand) to prevent FAS-mediated apoptosis of cells. Falcon 96-well non-tissue culture treated plates pre-coated overnight with anti-CD3 mAb at 500 ng/mL and costimulatory molecule B7H2 (B7 homolog 2) 5 μg/mL were washed and 100 μL of TIM-1 mAb treated Th1 or Th2 (200,000 cells) added per well. After 3 days of culture, the supernatants were removed and IL-4, IL-5, IL-10, IL-13, and IFNγ □□levels were determined by ELISA (Pharmingen, San Diego, Calif. or R&D Systems, Minneapolis, Minn.).

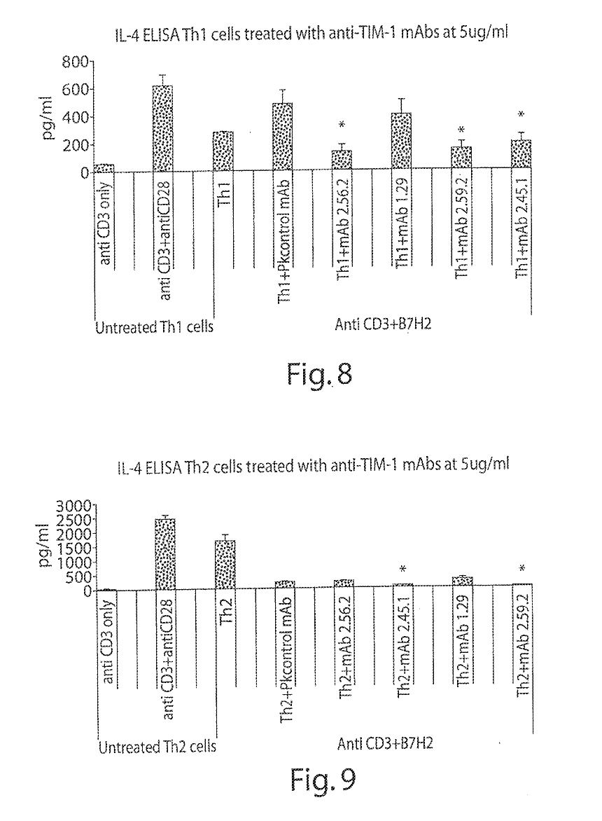

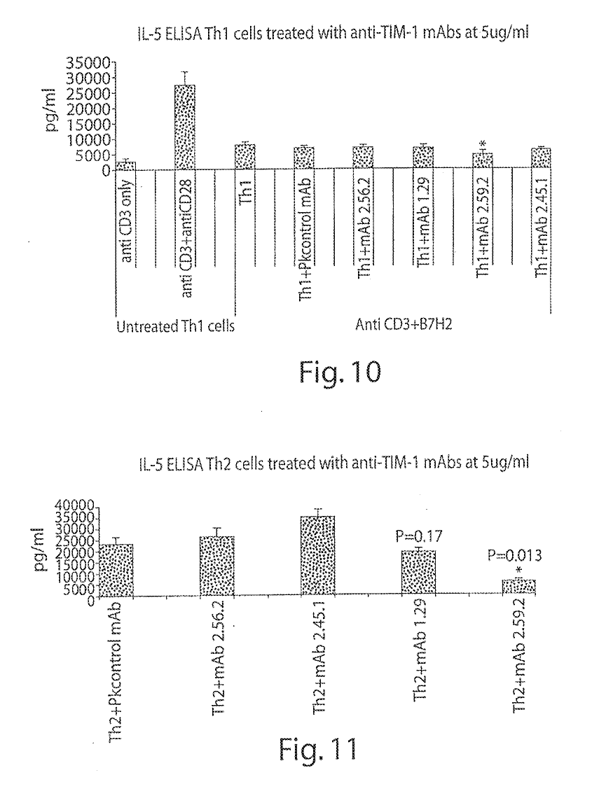

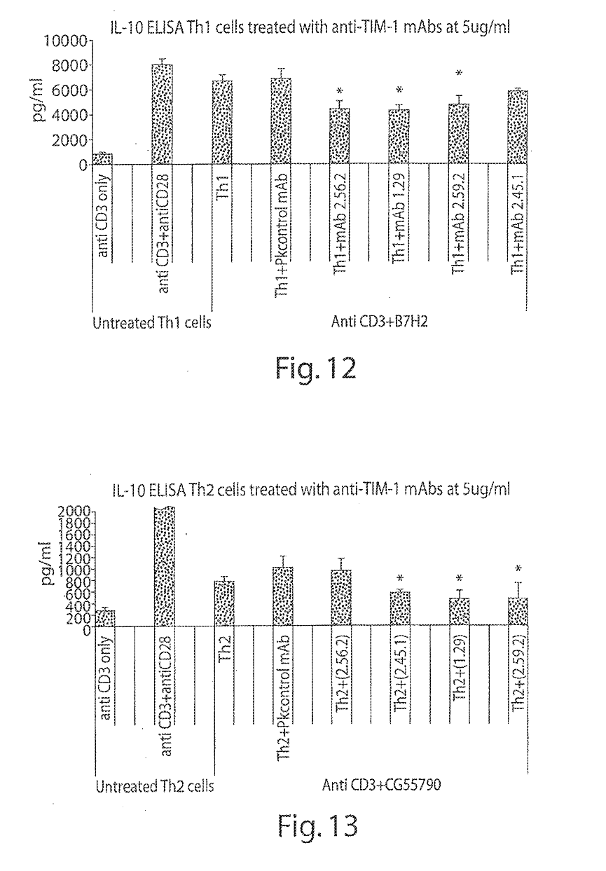

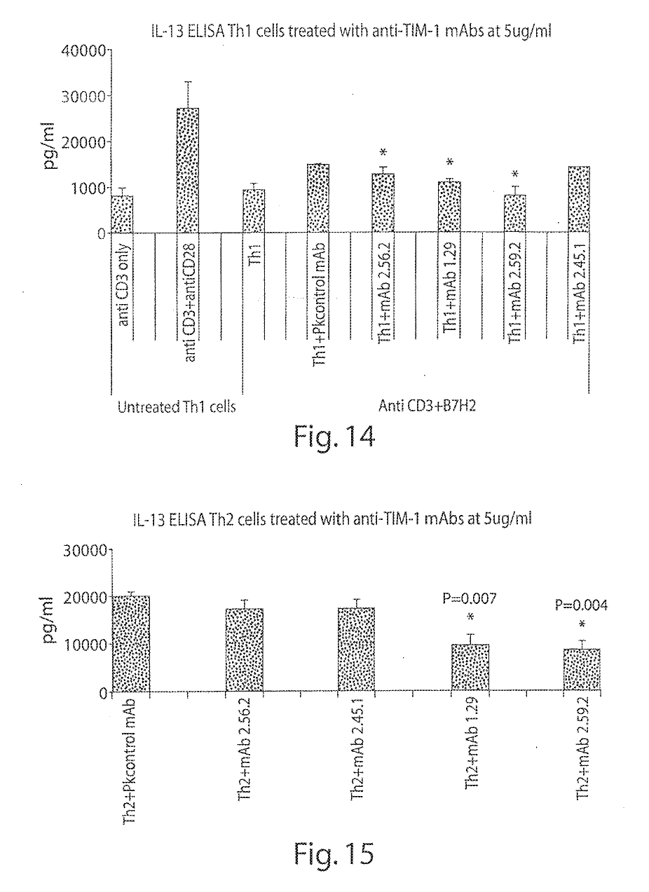

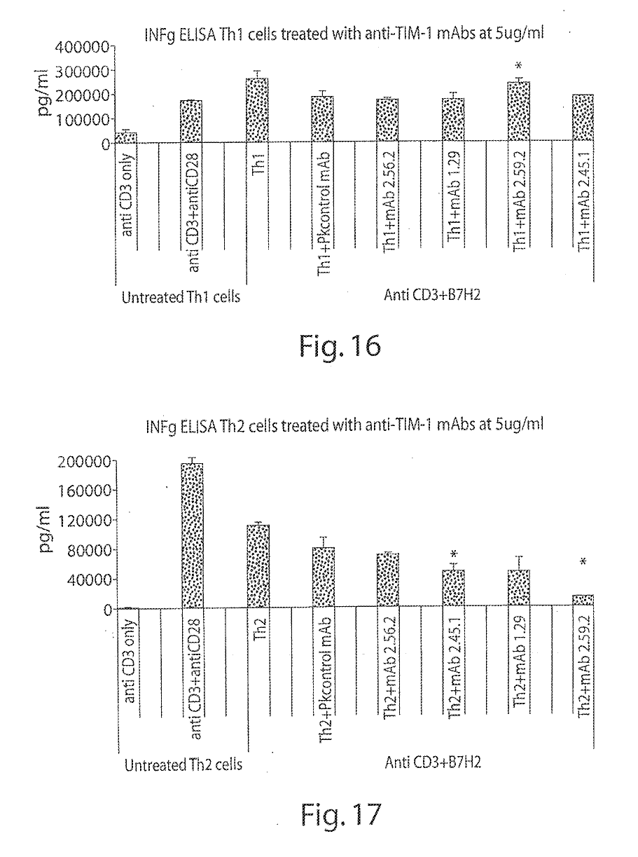

As demonstrated below, anti-TIM-1 mAb significantly inhibited release of the tested cytokines by Th1 and Th2 cells (see FIGS. 8-17). Results where inhibition of cytokine production is significant (p=0.02-0.008), are marked on the bar graphs with an asterisk. Tables 15 and 16 summarize the bar graphs in FIGS. 8-17.

TABLE 15
Cytokine Inhibition in CD4+ Thi cells using anti-TIM-1 antibodies
in two independent human donors

| Donor 12 + 17 | | | | | | |
|---|---|---|---|---|---|---|
| | Cytokines | | | | | |
| | Anti-TIM-1 | Percentage of Control Antibody | | | | |
| | mAbs | IL-5 | IL-4 | IL-10 | IL-13 | INF γ |
| TH1 | 2.56.2 | 100.17 | 28.49 * | 63.76 * | 86.45 | 93.69 |
| | 2.45.1 | 90.23 | 39.78 * | 83.98 | 96.25 | 100.6 |
| | 1.29 | 94.63 | 81.05 | 60.77 ** | 73.95 *** | 93.51 |
| | 2.59.2 | 66.62 * | 31.40 * | 68.99 * | 54.5 *** | 128.12 |

Experiments that demonstrate significant inhibition of cytokine production are marked with an asterisk:
P = 0.01 to 0.05 *;
P = 0.005 to 0.009 **;
P = 0.001 to 0.004 ***

TABLE 16
Cytokine Inhibition in CD4+ Th2 cellsusing anti-TIM-1 antibodies
in two independent human donors

| Donor 12 + 17 | | | | | | |
|---|---|---|---|---|---|---|
| | Cytokines | | | | | |
| | Anti-TIM-1 | Percentage of Control Antibody | | | | |
| | mAbs | IL-5 | IL-4 | IL-10 | IL-13 | INF γ |
| TH2 | 2.56.2 | 112.07 | 103.46 | 93.97 | 86.45 | 88.30 |
| | 2.45.1 | 148.7 | 25.66 *** | 55.97 * | 86.81 | 25.66 * |
| | 1.29 | 80.26 | 112.54 | 44.45 * | 48.91 ** | 112.54 |
| | 2.59.2 | 23.62 * | 19.17 ** | 43.86 * | 43.71 *** | 19.18 * |

Experiments that demonstrate significant inhibition of cytokine production are marked with an asterisk:
P = 0.01 to 0.05 *;
P = 0.005 to 0.009 **;
P = 0.001 to 0.004 ***

A summary of Th2 cytokine inhibition data obtained from multiple experiments with different donors is provided in Table 17. Each experiment used purified CD4+ cells isolated from whole blood samples from two independent donors. Cytokine production is reported as the percent of cytokine production detected using the control PK16.3 mAb. The anti-TIM-1 mAb used in each experiment is specified along the bottom row. Results that report significant cytokine inhibition are underlined in Table 17 below. The use of “ND” indicates that the experiment was not performed. These results do reflect donor dependent variability but show that mAbs 2.59.2 and 1.29 reproducibly block one or more of the Th2 cytokines.

TABLE 17
Summary of Cytokine Inhibition using anti-TIM-1 mAbs
2.59.2 and 1.29 in 5 independent human donor groups

| Donor ID | | | | | |
|---|---|---|---|---|---|
| Cytokine | 12 + 17 | 12 + 14 | 13 + 14 | 14 | 12 |
| IL-4 | 19 | 626 | 130 | ND | ND |
| IL-5 | 24 | 5 | 122 | 67 | 2 |
| IL-10 | 44 | 83 | 19 | 45 | 109 |
| IL-13 | 44 | ND | 17 | 100 | 91 |

| | |
|---|---|
| Anti-TIM-1 | Anti-TIM-1 mAb 1.29 |
| mAb 2.59.2 | |

Results of experiments that report inhibition greater than 50% of that seen using the control PK16.3 antibody are underlined.

### Example 13

### Construction, Expression and Purification of Anti-TIM-1 scFv

The VL and VH domains of mAb 2.70 were used to make a scFv construct. The sequence of the anti-TIM-1 scFv was synthesized by methods known in the art.

The nucleotide sequence of anti-TIM-1 scFv is as follows:

(SEQ ID NO: 108)
ATGAAATACCTGCTGCCGACCGCTGCTGCTGGTCTGCTGCTCCTCGCTGC
CCAGCCGGCCATGGCCGATATTGTGATGACCCAGACTCCACTCTCCCTGC
CCGTCACCCCTGGAGAGCCGGCCTCCATCTCCTGCAGGTCTAGTCGGAGC
CTCTTGGATAGTGATGATGGAAACACCTATTTGGACTGGTACCTGCAGAA
GCCAGGGCAGTCTCCACAGCTCCTGATCTACACGCTTTCCTATCGGGCCT
CTGGAGTCCCAGACAGGTTCAGTGGCAGTGGGTCAGGCACTGATTTCACA
CTGAAAATCAGCAGGGTGGAGGCTGAGGATGTTGGAGTTTATTACTGCAT
GCAACGTGTAGAGTTTCCTATCACCTTCGGCCAAGGGACACGACTGGAGA
TTAAACTTTCCGCGGACGATGCGAAAAAGGATGCTGCGAAGAAAGATGAC
GCTAAGAAAGACGATGCTAAAAAGGACCTCCAGGTGCAGCTGGTGGAGTC
TGGGGGAGGCGTGGTCCAGCCTGGGAGGTCCCTGAGACTCTCCTGTGCAG
CGTCTGGATTCATCTTCAGTCGCTATGGCATGCACTGGGTCCGCCAGGCT
CCAGGCAAGGGGCTGAAATGGGTGGCAGTTATATGGTATGATGGAAGTAA
TAAACTCTATGCAGACTCCGTGAAGGGCCGATTCACCATCTCCAGAGACA
ATTCCAAGAACACGCTGTATCTGCAAATGAACAGCCTGAGAGCCGAGGAC
ACGGCTGTGTATTACTGTGCGAGAGATTACTATGATAATAGTAGACATCA
CTGGGGGTTTGACTACTGGGGCCAGGGAACCCTGGTCACCGTCTCCTCAG
CTAGCGATTATAAGGACGATGATGACAAATAG

The amino acid sequence of mature anti-TIM-1 scFv is as follows:

(SEQ ID NO: 109)
DIVMTQTPLSLPVTPGEPASISCRSSRSLLDSDDGNTYLDWYLQKPGQSP
QLLIYTLSYRASGVPDRFSGSGSGTDFTLKISRVEAEDVGVYYCMQRVEF
PITFGQGTRLEIKLSADDAKKDAAKKDDAKKDDAKKDLQVQLVESGGGVV
QPGRSLRLSCAASGFIFSRYGMHWVRQAPGKGLKWVAVIWYDGSNKLYAD
SVKGRFTISRDNSKNTLYLQMNSLRAEDTAVYYCARDYYDNSRHHWGFDY
WGQGTLVTVSSASDYKDDDDK

The synthesized DNA can be inserted into the pET-20b(+) expression vector, for periplasmic expression in E. coli. Cells are grown and the periplasmic proteins prepared using standard protocols. Purification of the anti-TIM-1 scFv is achieved using an anti-FLAG M2 affinity column as per the manufacturer's directions. The predicted molecular weight of the mature protein is 30222.4 daltons. This purified scFv is used in the assays described below to test for biological activity. The scFv construct is comprised of a signal peptide (SP), VL (VL1) derived from mAb 2.70, a linker (L4) based on the 25 amino acid linker 205C, the VH (VH1) derived from mAb 2.70, and a Tag (in this case the FLAG tag). It will be obvious to those skilled in the art that other SP, linker and tag sequences could be utilized to get the same activity as the anti-TIM-1 scFv antibody described herein.

### Example 14

### Construction, Expression and Purification of Anti-TIM-1 and Anti-CD3 Bispecific scFv1

The basic formula for the construction of this therapeutic protein is as follows:
SP1-VL1-L1-VH1-L2-VH2-L3-VL2-Tag

The signal peptide SP1 is the same as IgG kappa signal peptide VKIII A27 from Medical Research Council (MRC) Centre for Protein Engineering, University of Cambridge, UK.

Other signal peptides can also be used and will be obvious to those skilled in the art. This protein is designed to be expressed from mammalian cells. The predicted molecular weight of the mature cleaved protein is 54833.3 dalton. L1 corresponds to the (Gly4Ser)3 linker, while linker 2 (L2) corresponds to the short linker sequence: GGGGS. L3 is an 18 amino acid linker. VH2 corresponds to the anti-CD3 variable heavy chain domain from Genbank (accession number CAE85148) while VL1 corresponds to the anti-CD3 variable light chain domain from Genbank (accession number CAE85148). The tag being used for this construct is a His tag to facilitate purification and detection of this novel protein. Standard protocols are used to express and purify this His tagged protein, which is tested for activity and tumor cell killing in the protocols described below.

The amino acid and nucleic acid numbering for the components comprising the anti-TIM-1 and anti-CD3 bispecific scFv1 is as follows:
SP: −20 to −1 aa; −60 to −1 nt
VL1: 1-113 aa; 1-339nt
L1: 114-128 aa; 340-384nt
VH1: 129-251 aa; 385-753nt
L2: 252-256 aa; 754-768nt
VH2: 257-375 aa; 769-1125nt
L3: 376-393 aa; 1126-1179nt
VL2: 394-499 aa; 1180-1497nt
Tag: 500-505 aa; 1498-1515nt

The nucleotide sequence of anti-TIM-1 and anti-CD3 bispecific scFv1 is as follows:

(SEQ ID NO: 110)
ATGGAAACCCCAGCGCAGCTTCTCTTCCTCCTGCTACTCTGGCTCCCAGA
TACCACCGGAGATATTGTGATGACCCAGACTCCACTCTCCCTGCCCGTCA
CCCCTGGAGAGCCGGCCTCCATCTCCTGCAGGTCTAGTCGGAGCCTCTTG
GATAGTGATGATGGAAACACCTATTTGGACTGGTACCTGCAGAAGCCAGG
GCAGTCTCCACAGCTCCTGATCTACACGCTTTCCTATCGGGCCTCTGGAG
TCCCAGACAGGTTCAGTGGCAGTGGGTCAGGCACTGATTTCACACTGAAA
ATCAGCAGGGTGGAGGCTGAGGATGTTGGAGTTTATTACTGCATGCAACG
TGTAGAGTTTCCTATCACCTTCGGCCAAGGGACACGACTGGAGATTAAAG
GTGGTGGTGGTTCTGGCGGCGGCGGCTCCGGTGGTGGTGGTTCCCAGGTG
CAGCTGGTGGAGTCTGGGGGAGGCGTGGTCCAGCCTGGGAGGTCCCTGAG
ACTCTCCTGTGCAGCGTCTGGATTCATCTTCAGTCGCTATGGCATGCACT
GGGTCCGCCAGGCTCCAGGCAAGGGGCTGAAATGGGTGGCAGTTATATGG
TATGATGGAAGTAATAAACTCTATGCAGACTCCGTGAAGGGCCGATTCAC
CATCTCCAGAGACAATTCCAAGAACACGCTGTATCTGCAAATGAACAGCC
TGAGAGCCGAGGACACGGCTGTGTATTACTGTGCGAGAGATTACTATGAT
AATAGTAGACATCACTGGGGGTTTGACTACTGGGGCCAGGGAACCCTGGT
CACCGTCTCCTCAGGAGGTGGTGGATCCGATATCAAACTGCAGCAGTCAG
GGGCTGAACTGGCAAGACCTGGGGCCTCAGTGAAGATGTCCTGCAAGACT
TCTGGCTACACCTTTACTAGGTACACGATGCACTGGGTAAAACAGAGGCC
TGGACAGGGTCTGGAATGGATTGGATACATTAATCCTAGCCGTGGTTATA
CTAATTACAATCAGAAGTTCAAGGACAAGGCCACATTGACTACAGACAAA
TCCTCCAGCACAGCCTACATGCAACTGAGCAGCCTGACATCTGAGGACTC
TGCAGTCTATTACTGTGCAAGATATTATGATGATCATTACTGCCTTGACT
ACTGGGGCCAAGGCACCACTCTCACAGTCTCCTCAGTCGAAGGTGGAAGT
GGAGGTTCTGGTGGAAGTGGAGGTTCAGGTGGAGTCGACGACATTCAGCT
GACCCAGTCTCCAGCAATCATGTCTGCATCTCCAGGGGAGAAGGTCACCA
TGACCTGCAGAGCCAGTTCAAGTGTAAGTTACATGAACTGGTACCAGCAG
AAGTCAGGCACCTCCCCCAAAAGATGGATTTATGACACATCCAAAGTGGC
TTCTGGAGTCCCTTATCGCTTCAGTGGCAGTGGGTCTGGGACCTCATACT
CTCTCACAATCAGCAGCATGGAGGCTGAAGATGCTGCCACTTATTACTGC
CAACAGTGGAGTAGTAACCCGCTCACGTTCGGTGCTGGGACCAAGCTGGA
GCTGAAATAG

The protein sequence of mature anti-TIM-1 and anti-CD3 bispecific scFv1 is as follows:

(SEQ ID NO: 111)
DIVMTQTPLSLPVTPGEPASISCRSSRSLLDSDDGNTYLDWYLQKPGQSP
QLLIYTLSYRASGVPDRFSGSGSGTDFTLKISRVEAEDVGVYYCMQRVEF
PITFGQGTRLEIKGGGGSGGGGSGGGGSQVQLVESGGGVVQPGRSLRLSC
AASGFIFSRYGMHWVRQAPGKGLKWVAVIWYDGSNKLYADSVKGRFTISR
DNSKNTLYLQMNSLRAEDTAVYYCARDYYDNSRHHWGFDYWGQGTLVTVS
SGGGGSDIKLQQSGAELARPGASVKMSCKTSGYTFTRYTMHWVKQRPGQG
LEWIGYINPSRGYTNYNQKFKDKATLTTDKSSSTAYMQLSSLTSEDSAVY
YCARYYDDHYCLDYWGQGTTLTVSSVEGGSGGSGGSGGSGGVDDIQLTQS
PAIMSASPGEKVTMTCRASSSVSYMNWYQQKSGTSPKRWIYDTSKVASGV
PYRFSGSGSGTSYSLTISSMEAEDAATYYCQQWSSNPLTFGAGTKLELK

### Example 15

### Construction, Expression and Purification of Anti-TIM-1 and Anti-CD3 Bispecific scFv2

The basic formula for the construction of this novel therapeutic protein is as follows:
SP1-VL1-L4-VH1-L2-VH2-L4-VL2-Tag

The signal peptide SP1 is IgG kappa signal peptide VKIII A27 from Medical Research Council (MRC) Centre for Protein Engineering, University of Cambridge, UK. For more information see mrc-cpe.cam.ac.uk/ALIGNMENTS.php?menu=901. Other signal peptides and linkers could also be used to get additional biologically active bispecific single chain antibodies. The protein being described in this example is also designed to be expressed from mammalian cells and is similar to the anti-TIM-1 and anti-CD3 bispecific scFv1, except that it utilizes a different linker as indicated in the basic formula above (L4, as described earlier), and that a Flag tag is used instead of the His tag as in the first example.

The predicted molecular weight of the mature cleaved protein is 58070.0 dalton. The tag being used for this construct is a FLAG tag to facilitate purification and detection of this novel protein. Standard protocols are used to express this secreted protein and purify it, which is tested for activity and tumor cell killing in the protocols described below.

The amino acid and nucleic acid numbering for the components comprising the anti-TIM-1 and anti-CD3 bispecific scFv2 is as follows:
SP: −20 to −1 aa; −60 to −1nt
VL1: 1-113 aa; 1-339nt
L1: 114-138 aa; 340-414nt
VH1: 139-261 aa; 415-783nt
L2: 262-266 aa; 784-798nt
VH2: 267-385 aa; 799-1155nt
L3: 386-410 aa; 1156-1230nt
VL2: 411-516 aa; 1231-1548nt
Tag: 517-524 aa; 1549-1572nt

The nucleotide sequence of anti-TIM-1 and anti-CD3 bispecific scFv2 is as follows:

(SEQ ID NO: 112)
ATGGAAACCCCAGCGCAGCTTCTCTTCCTCCTGCTACTCTGGCTCCCAGA
TACCACCGGAGATATTGTGATGACCCAGACTCCACTCTCCCTGCCCGTCA
CCCCTGGAGAGCCGGCCTCCATCTCCTGCAGGTCTAGTCGGAGCCTCTTG
GATAGTGATGATGGAAACACCTATTTGGACTGGTACCTGCAGAAGCCAGG
GCAGTCTCCACAGCTCCTGATCTACACGCTTTCCTATCGGGCCTCTGGAG
TCCCAGACAGGTTCAGTGGCAGTGGGTCAGGCACTGATTTCACACTGAAA
ATCAGCAGGGTGGAGGCTGAGGATGTTGGAGTTTATTACTGCATGCAACG
TGTAGAGTTTCCTATCACCTTCGGCCAAGGGACACGACTGGAGATTAAAC
TTTCCGCGGACGATGCGAAAAAGGATGCTGCGAAGAAAGATGACGCTAAG
AAAGACGATGCTAAAAAGGACCTGCAGGTGCAGCTGGTGGAGTCTGGGGG
AGGCGTGGTCCAGCCTGGGAGGTCCCTGAGACTCTCCTGTGCAGCGTCTG
GATTCATCTTCAGTCGCTATGGCATGCACTGGGTCCGCCAGGCTCCAGGC
AAGGGGCTGAAATGGGTGGCAGTTATATGGTATGATGGAAGTAATAAACT
CTATGCAGACTCCGTGAAGGGCCGATTCACCATCTCCAGAGACAATTCCA
AGAACACGCTGTATCTGCAAATGAACAGCCTGAGAGCCGAGGACACGGCT
GTGTATTACTGTGCGAGAGATTACTATGATAATAGTAGACATCACTGGGG
GTTTGACTACTGGGGCCAGGGAACCCTGGTCACCGTCTCCTCAGGAGGTG
GTGGATCCGATATCAAACTGCAGCAGTCAGGGGCTGAACTGGCAAGACCT
GGGGCCTCAGTGAAGATGTCCTGCAAGACTTCTGGCTACACCTTTACTAG
GTACACGATGCACTGGGTAAAACAGAGGCCTGGACAGGGTCTGGAATGGA
TTGGATACATTAATCCTAGCCGTGGTTATACTAATTACAATCAGAAGTTC
AAGGACAAGGCCACATTGACTACAGACAAATCCTCCAGCACAGCCTACAT
GCAACTGAGCAGCCTGACATCTGAGGACTCTGCAGTCTATTACTGTGCAA
GATATTATGATGATCATTACTGCCTTGACTACTGGGGCCAAGGCACCACT
CTCACAGTCTCCTCACTTTCCGCGGACGATGCGAAAAAGGATGCTGCGAA
GAAAGATGACGCTAAGAAAGACGATGCTAAAAAGGACCTGGACATTCAGC
TGACCCAGTCTCCAGCAATCATGTCTGCATCTCCAGGGGAGAAGGTCACC
ATGACCTGCAGAGCCAGTTCAAGTGTAAGTTACATGAACTGGTACCAGCA
GAAGTCAGGCACCTCCCCCAAAAGATGGATTTATGACACATCCAAAGTGG
CTTCTGGAGTCCCTTATCGCTTCAGTGGCAGTGGGTCTGGGACCTCATAC
TCTCTCACAATCAGCAGCATGGAGGCTGAAGATGCTGCCACTTATTACTG
CCAACAGTGGAGTAGTAACCCGCTCACGTTCGGTGCTGGGACCAAGCTGG
AGCTGAAAGATTATAAGGACGATGATGACAAATAG

The protein sequence of mature anti-TIM-1 and anti-CD3 bispecific scFv2 is as follows:

(SEQ ID NO: 113)
DIVMTQTPLSLPVTPGEPASISCRSSRSLLDSDDGNTYLDWYLQKPGQSP
QLLIYTLSYRASGVPDRFSGSGSGTDFTLKISRVEAEDVGVYYCMQRVEF
PITFGQGTRLEIKLSADDAKKDAAKKDDAKKDDAKKDLQVQLVESGGGVV
QPGRSLRLSCAASGFIFSRYGMHWVRQAPGKGLKWVAVIWYDGSNKLYAD
SVKGRFTISRDNSKNTLYLQMNSLRAEDTAVYYCARDYYDNSRHHWGFDY
WGQGTLVTVSSGGGGSDIKLQQSGAELARPGASVKMSCKTSGYTFTRYTM
HWVKQRPGQGLEWIGYINPSRGYTNYNQKFKDKATLTTDKSSSTAYMQLS
SLTSEDSAVYYCARYYDDHYCLDYWGQGTTLTVSSLSADDAKKDAAKKDD
AKKDDAKKDLDIQLTQSPAIMSASPGEKVTMTCRASSSVSYMNWYQQKSG
TSPKRWIYDTSKVASGVPYRFSGSGSGTSYSLTISSMEAEDAATYYCQQW
SSNPLTFGAGTKLELKDYKDDDDK

### Example 16

### Anti-TIM-1 scFv Species Biological Activity

### ELISA Analysis:

To determine if the anti-TIM-1 and anti-CD3 bispecific scFv1 and scFv2 antibodies bind to specific antigen, ELISA analysis is performed. 1 μg/ml of specific antigen (TIM-1 antigen (CG57008-02) is bound to ELISA plates overnight in carbonate/bicarbonate buffer (pH approximately 9.2-9.4). Plates are blocked with assay diluent buffer purchased from Pharmingen San Diego, Calif.), and various concentrations of the anti-TIM-1 scFv bispecific antibodies are added for 1 hour at room temp. Plates are washed in 0.01% Tween 20 in PBS, followed by addition of HRP-conjugated mAb to either the 6-His tag (Invitrogen, Carlsbad, Calif.) or the FLAG peptide tag or (Sigma, St. Louis, Mo.) in assay diluent for 60 minutes at room temperature. Color is developed with TMB substrate (Pharmingen), and the reaction stopped with H₂SO₄. Plates are read at A450 nm, and the O.D. value taken as a measure of protein binding.

### FACS Analysis

Binding of the anti-TIM-1 and anti-CD3 bispecific scFv1 and scFv2 antibodies, as well as the anti-TIM-1 scFv antibody to cells expressing the antigens recognized by the anti-TIM-1 human mAbs is examined by FACS analysis. Cells (such as ACHN) are washed in PBS and resuspended in FACS buffer consisting of ice cold PBS with addition of 1% BSA or 1% FBS. The resuspended cells are then incubated on ice with various concentrations of the bispecific antibody for 30 minutes. Cells are washed to remove non-bound antibody. Bound antibody is detected by binding of a secondary labeled mAb (phycoerythrin or FITC labeled) that specifically recognizes the 6-his tag or the FLAG-tag that is engineered on the bispecific antibody sequence. Cells are washed and analyzed for binding of the anti-tag mAb by FACS analysis. Binding of bispecific mAb plus anti-tag mAb is compared to binding of the anti-tag mAb alone.

### Cytotoxicity Analysis

To determine if the bispecific antibody has functional activity as defined by the ability of the bispecific to target T cells to TIM-1 expressing normal or tumor cells, the bispecific antibody is tested in a Cytotoxicity assay. T cells are obtained from the low density cells derived from centrifugation of blood over density separation medium (specific density 1.077). T cells can be used in a heterogeneous mix from the peripheral blood mononuclear cell fraction (which also contains B cells, NK cells and monocytes) or further purified from the low-density cells using MACS separation and negative or positive selection. Killing in assays with T cells derived from the blood directly will have less cytolytic activity than cells that have been stimulated in vitro with PHA, cytokines, activating monoclonal antibodies or other stimulators of polyclonal T cell activation. Therefore, these activators will be used to further boost the activity of T cells in the functional assays. Many variations of cytotoxicity assays are available. Cytotoxicity assays measure the release of natural products of the cells metabolism upon lysis, such as LDH. Other assays are based around labeling cells with various agents such as radioactive chromium (51Cr), DELFIA BATDA, CSFE or similar labeling agents and detecting release or change in live cells bound by the agent.

DELFIA cytotoxicity assays (PerkinElmer Life and Analytical Sciences, Inc. Boston, Mass.) offer a non-radioactive method to be used in cell mediated cytotoxicity studies. The method is based on loading cells with an acetoxymethyl ester of a fluorescence enhancing ligand. After the ligand has penetrated the cell membrane the ester bonds are hydrolyzed within the cell to form a hydrophilic ligand, which no longer passes through the membrane. After cytolysis the released ligand is introduced to a europium solution to form a fluorescent chelate. The measured signal correlates directly with the amount of lysed cells. Target cells are resuspended to a concentration of 2×10⁶/ml. 10 μl of DELFIA BATDA was mixed in a tube with 2 ml of target cells according to the manufacturers instructions. Various concentrations of T cells are added to a fixed concentration of labeled target cells (5000 cells per well) in 96 well U-bottom plates, and incubated for at least 2 hours at 37° C. The plates are spun at approximately 200 g, followed by the aspiration of 20 μl of supernatant, which was then added to a europium solution (200 μl) in a separate plate. The plate is incubated for 15 minutes at room temperature, followed by analysis on a SAFIRE (Tecan, Maennedorf, Switzerland) according to the manufacturer's instructions. Signal in the test wells are compared to signal in 100% lysis well (10% lysis buffer in place of T cells) and cell with medium alone (spontaneous release), and % specific lysis is calculated from the formula

% specific lysis=(test−spontaneous release)/100% lysis×100.

BIAcore Kinetic Analysis of scFv Constructs

Kinetic measurements to determine the affinity for the scFv constructs (monomer as well as bispecific, containing at least 1 scFv moiety binding to TIM-1) are measured using the methods described earlier for the whole antibodies of this invention. scFv-containing antibody protein affinities to TIM-1 are expected to be within a factor of 10, i.e. between 0.271-27.1 nM, of the affinity given for mAb 2.70.

### Example 17

### Ability of Anti-TIM-1 mAb to Inhibit the Proliferation of Human Ovary Carcinoma Cells

Several fully human monoclonal antibody clones were isolated from the immunizations described above and their ability to inhibit the proliferative potential of OVCAR-5 (human ovary carcinoma) cells was analyzed using the 5-bromo-2-deoxyuridine (BrdU) incorporation assay (described in International Patent Application No. WO 01/25433).

In the BrdU assay, OVCAR-5 cancer cells (Manassas, Va.) were cultured in Dulbeccos Modification of Eagles Medium (DMEM) supplemented with 10% fetal bovine serum or 10% calf serum respectively. The ovarian cancer cell line was grown to confluence at 37° C. in 10% CO₂/air. Cells were then starved in DMEM for 24 hours. Enriched conditioned medium was added (10 μL/100 μL of culture) for 18 hours. BrdU (10 μM) was then added and incubated with the cells for 5 hours. BrdU incorporation was assayed by colorimetric immunoassay according to the manufacturer's specifications (Boehringer Mannheim, Indianapolis, Ind.).

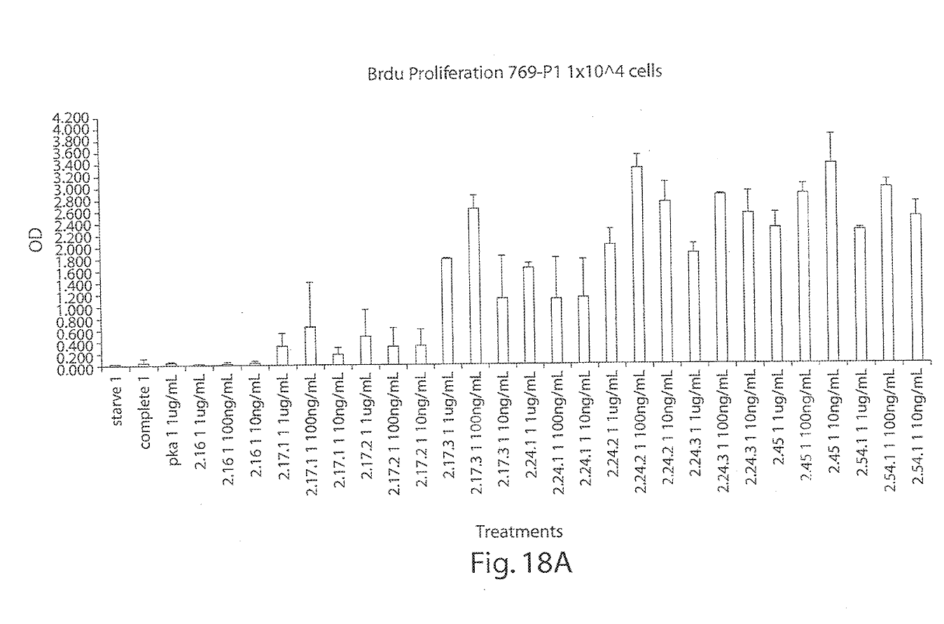

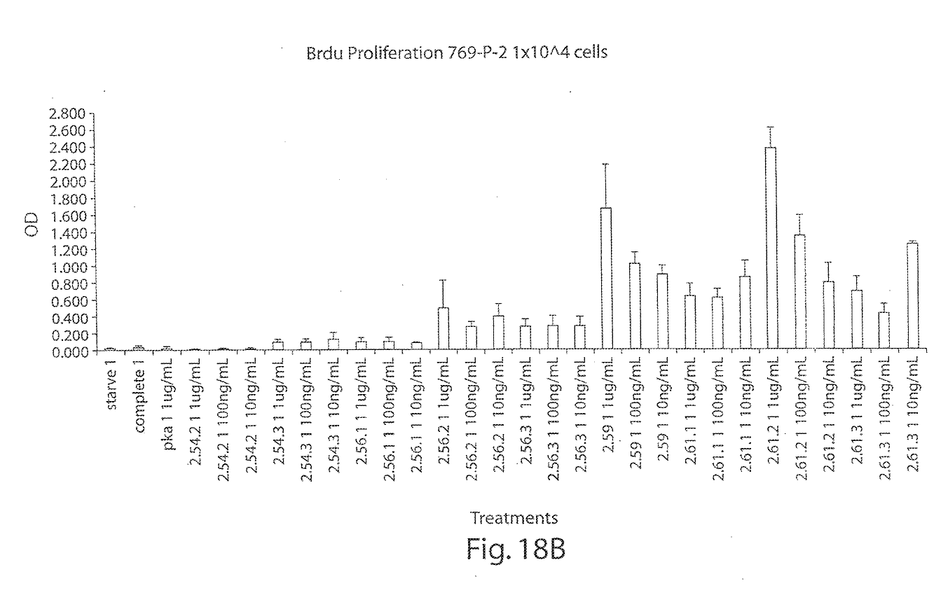

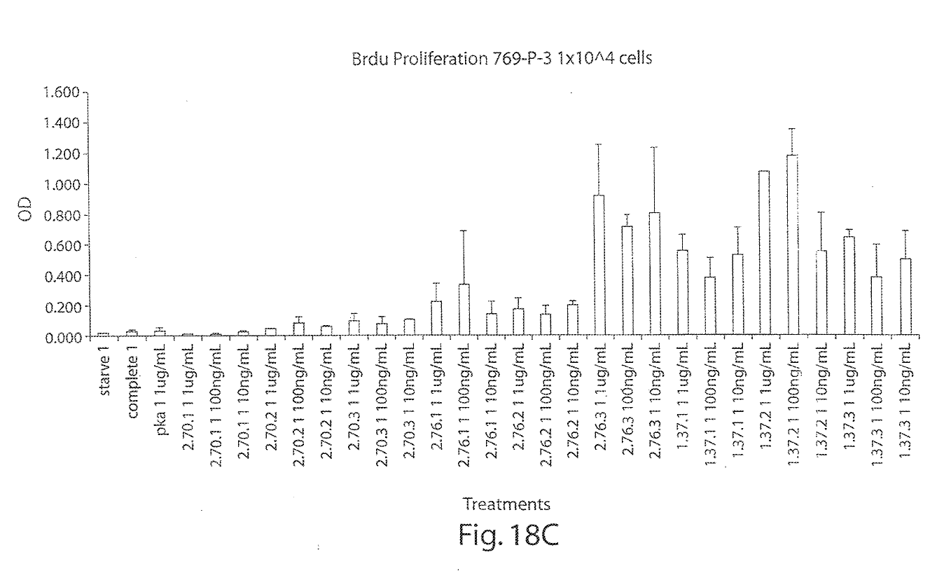

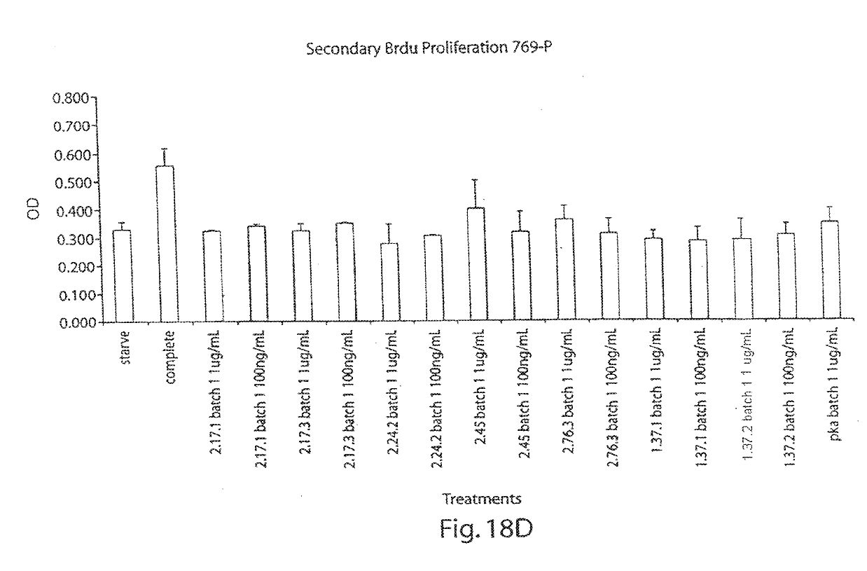

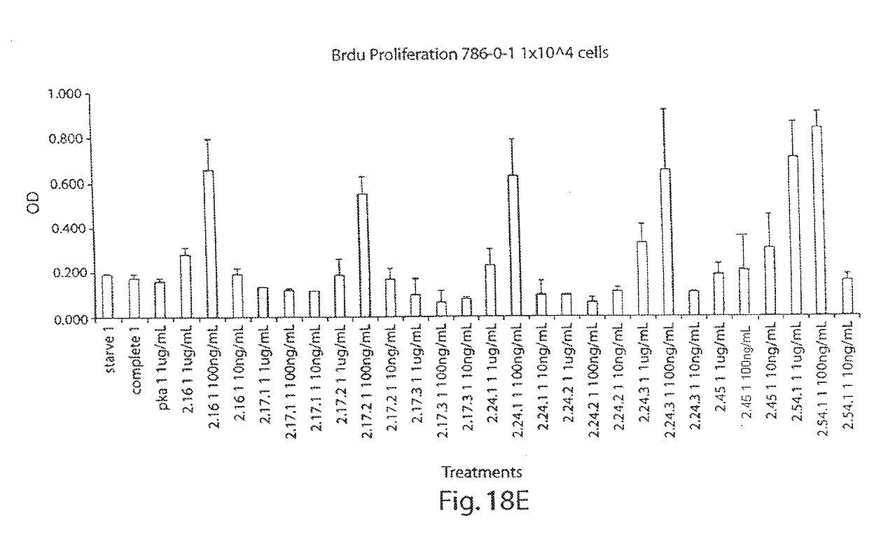

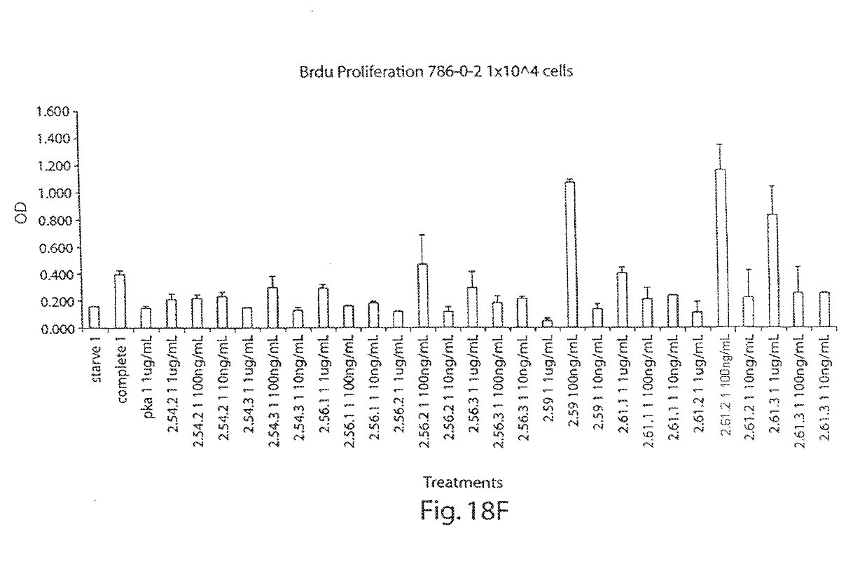

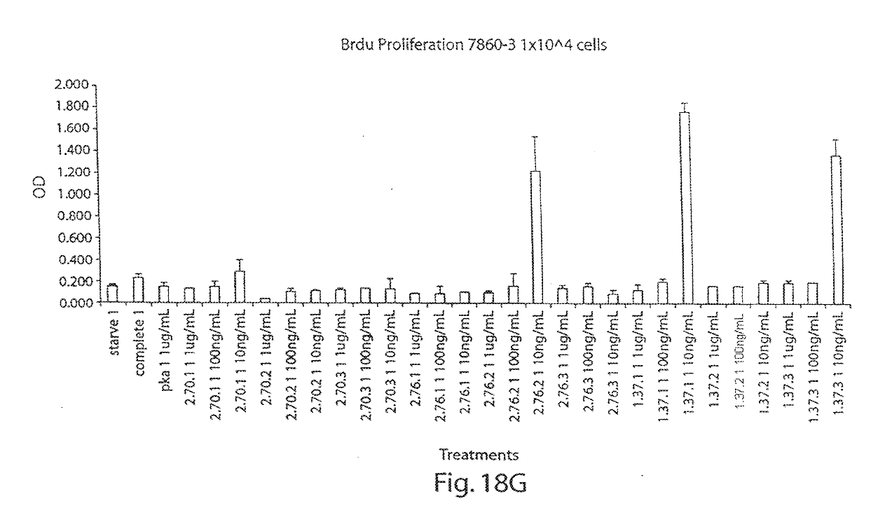

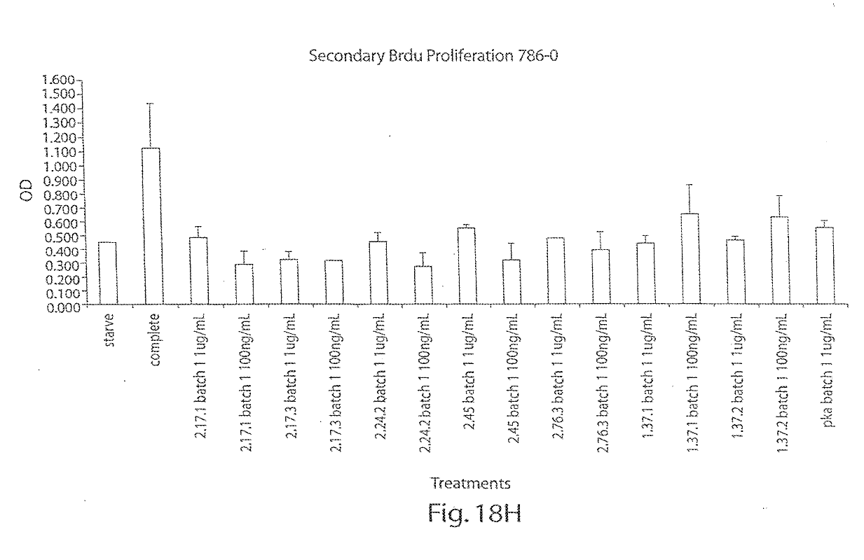

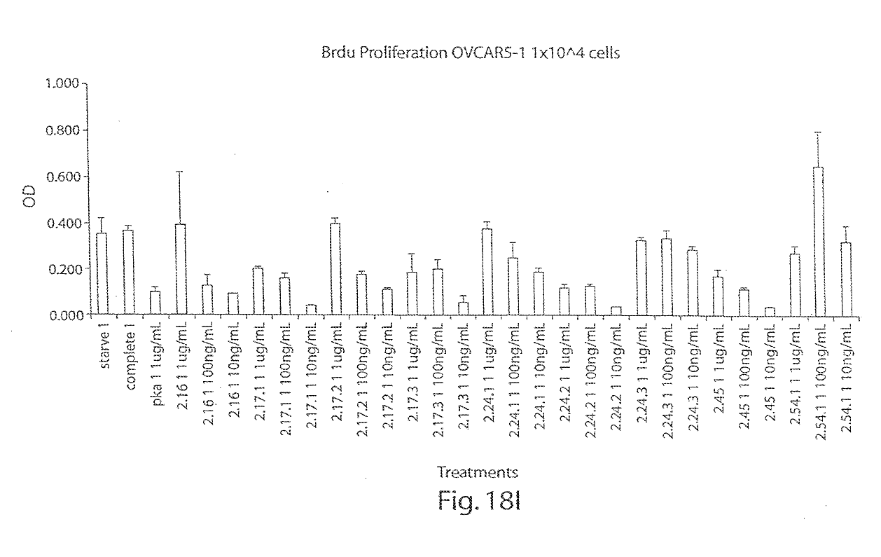

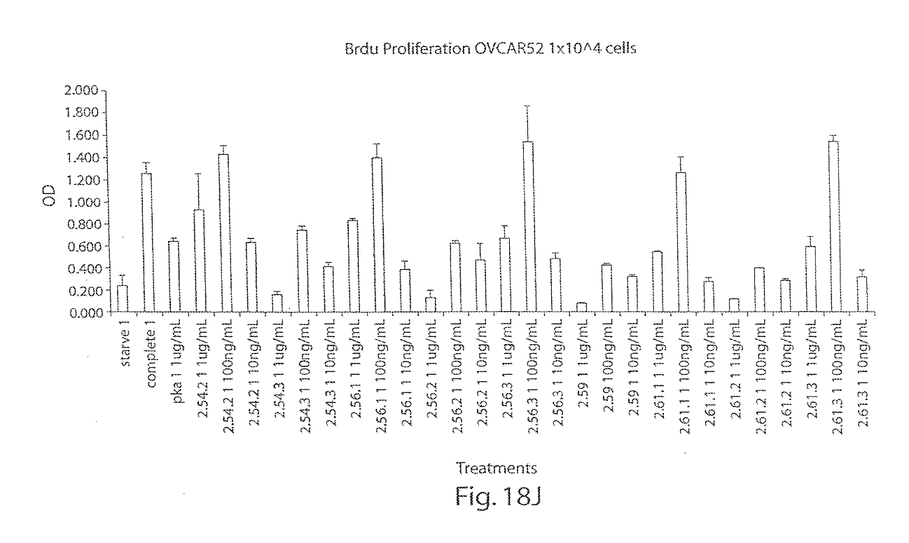

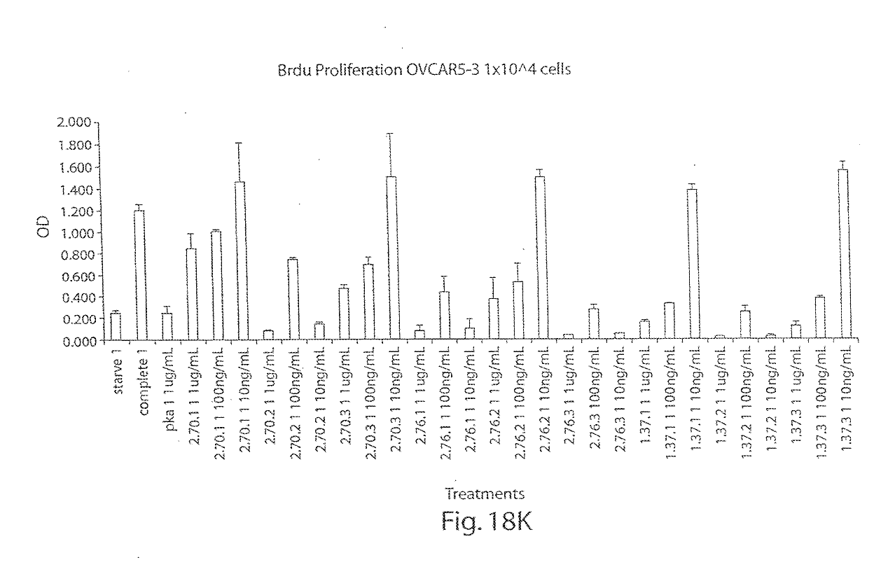

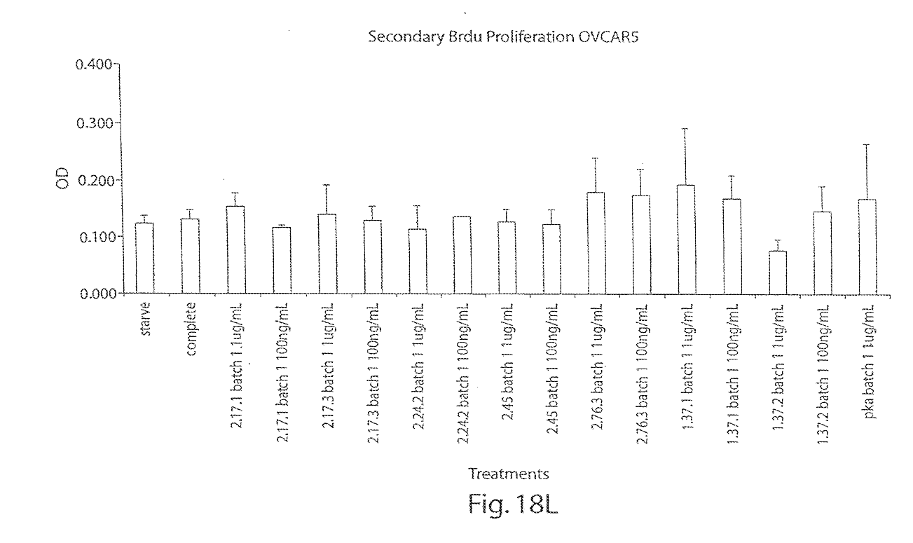

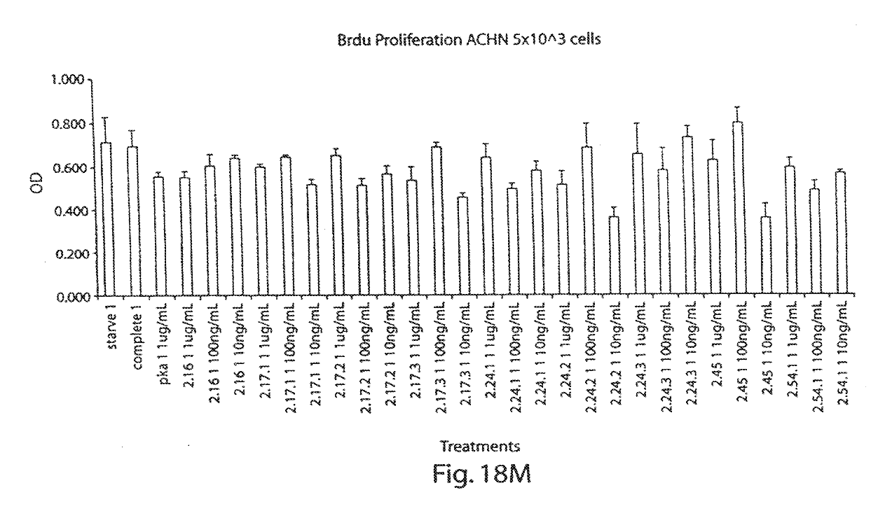

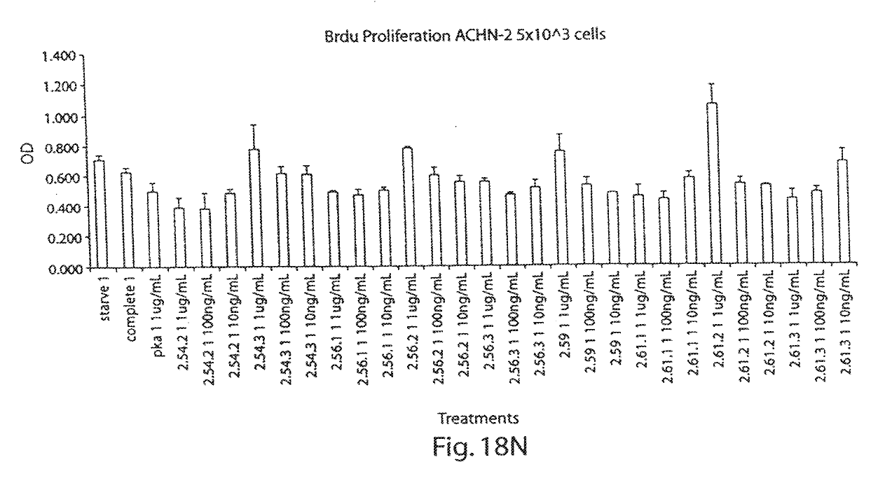

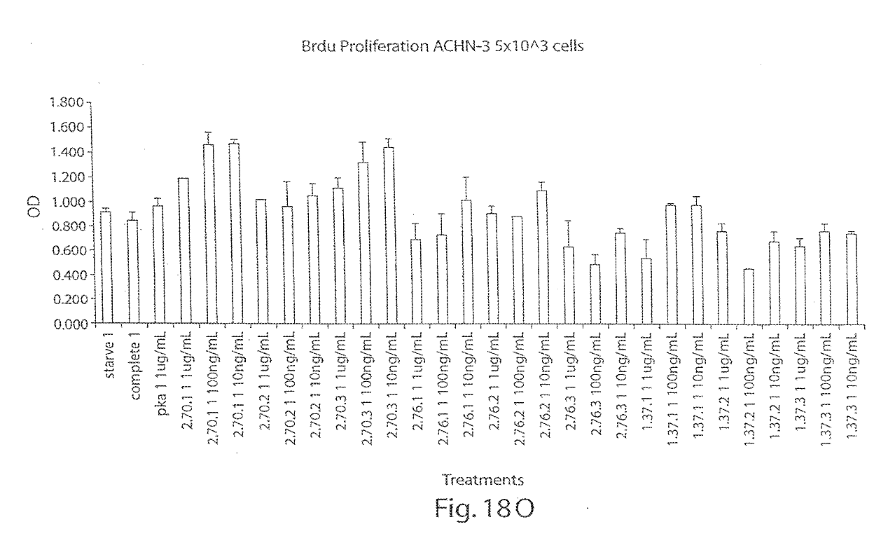

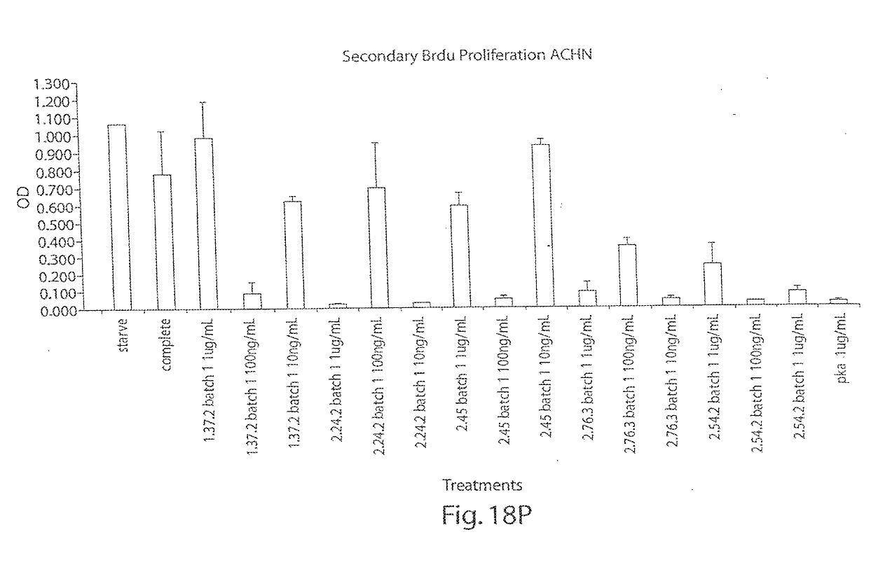

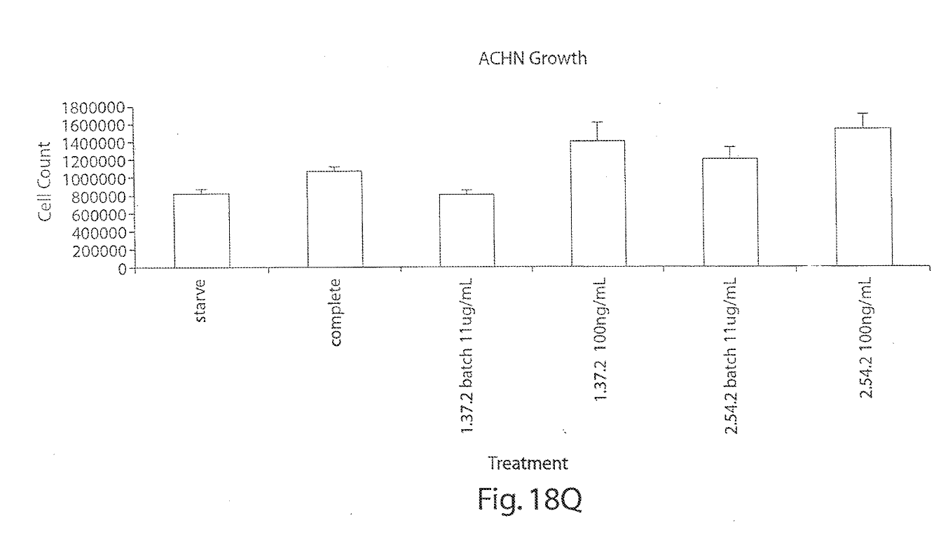

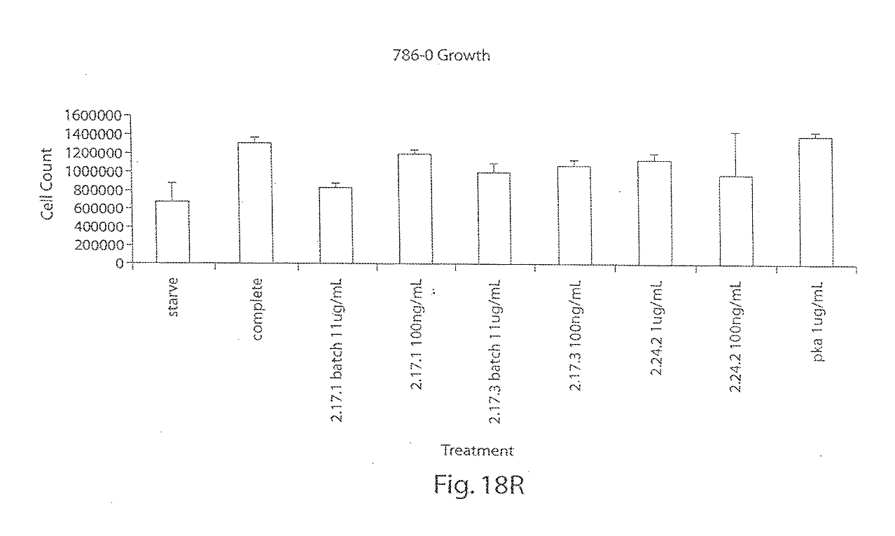

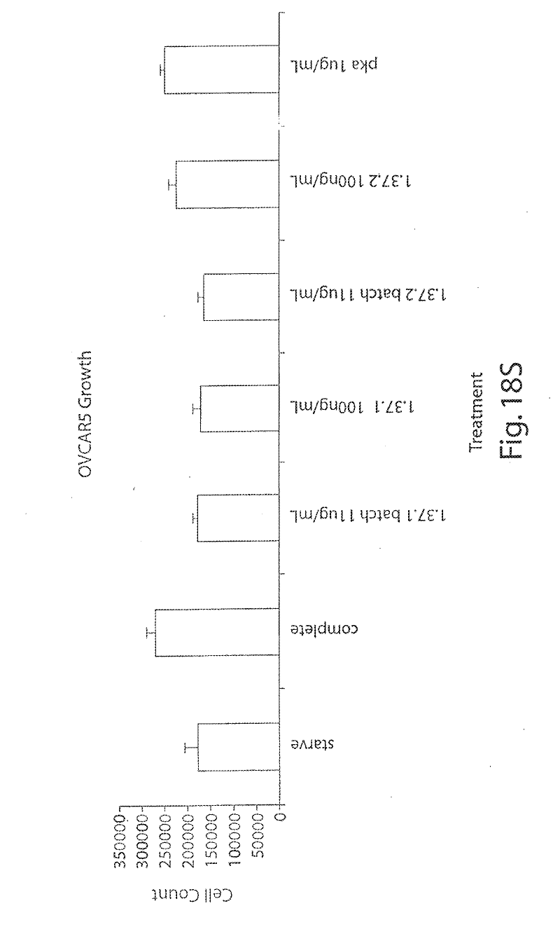

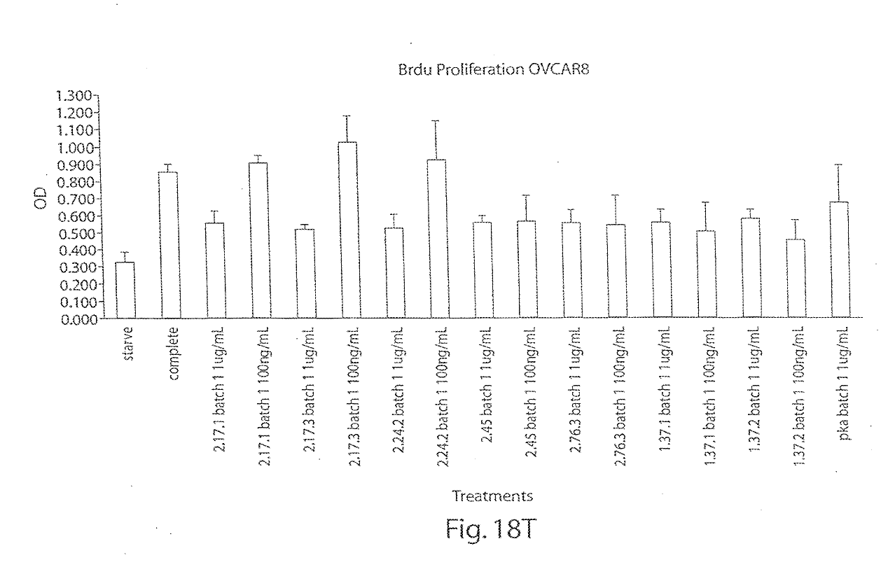

The capability of various human anti-TIM-1 monoclonal antibodies to neutralize was assessed. The results provided in FIGS. 18A-18T are presented in a bar graph format to assist in comparing the levels of BrdU incorporation in OVCAR5 cells upon exposure to various human anti-TIM-1monoclonal antibodies described herein. As positive and negative controls, OVCAR5 cells were cultured in the presence of either complete media (complete) or restricted serum-containing media (starved). In addition, the monoclonal antibody PK16.3 was included as a negative treatment control representing a human IgG antibody of irrelevant specificity. Human anti-TIM-1 monoclonal antibodies described herein were used at varying doses (10-1000 ng/mL) as compared to a control run utilizing varying concentrations.

### Example 18

### Antibody Conjugate Studies

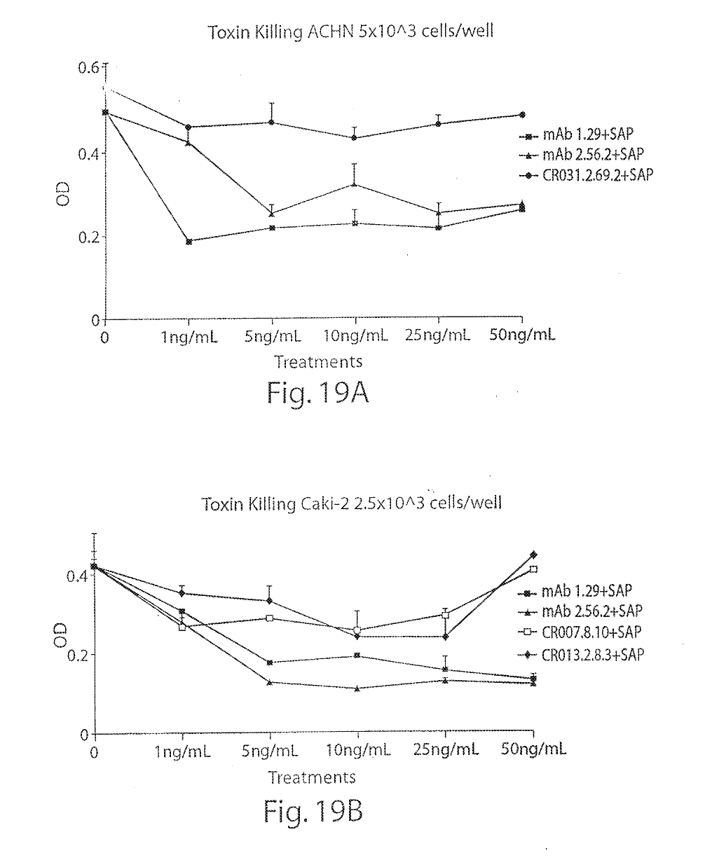

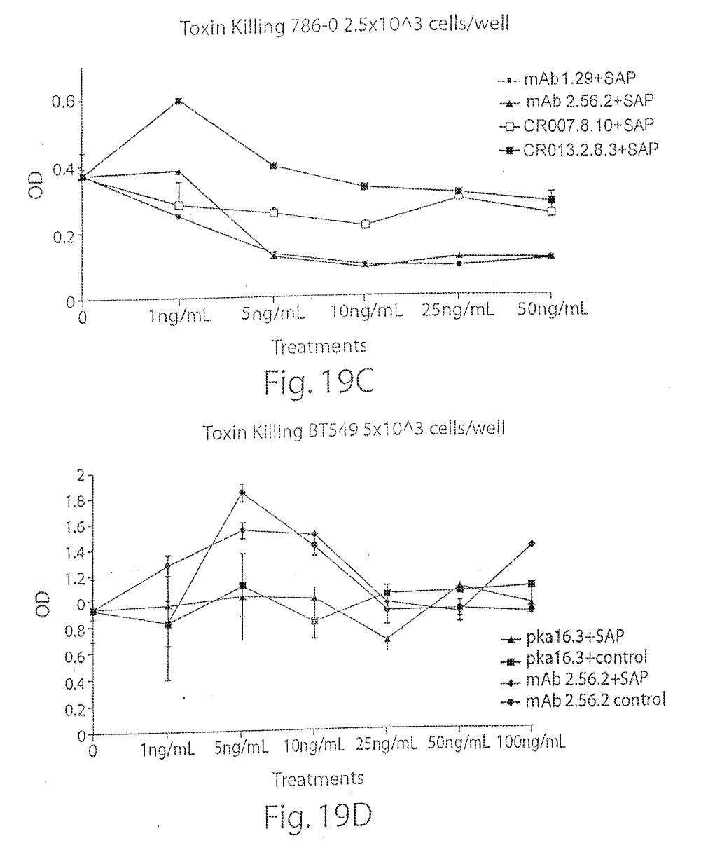

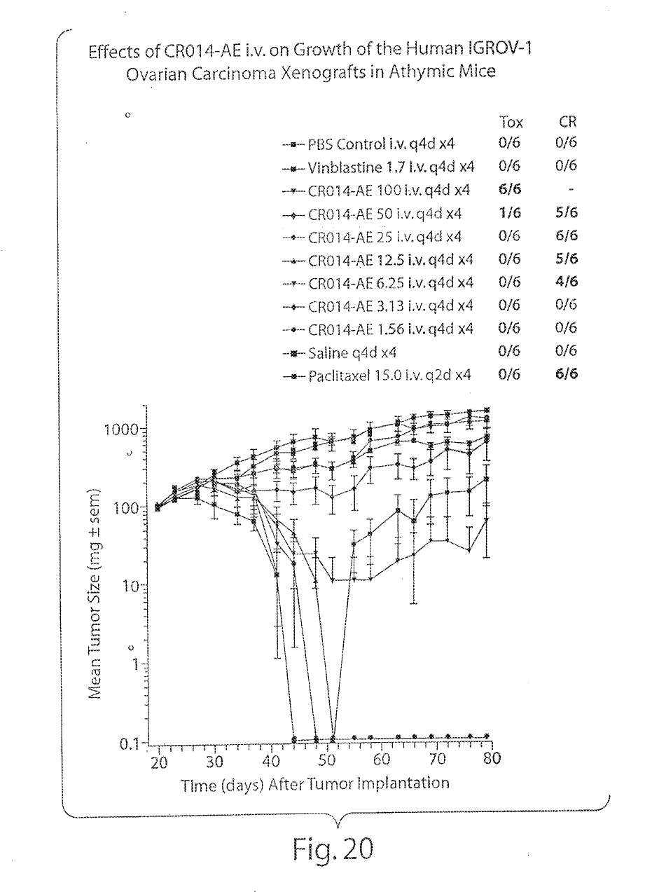

Additional antibody conjugate studies were performed using the plant toxin saporin conjugated to anti-TIM-1-specific mABs (1.29 and 2.56.2) and various irrelevant antibodies, including, PK16.3 (FIGS. 19A-19C). Additional negative controls included anti-TIM-1-specific mAB 2.56.2 and irrelevant antibody PK16.3 without toxin (FIG. 19D). Four cancer cell lines, three kidney cancer cell lines (ACHN, CAKI, and 7860) and one breast cancer cell line (BT549), were treated for 72 hours with saporin-antibody conjugates or antibodies alone, after which time BrdU was added to monitor proliferation over a 24 hour period. The results are described in FIGS. 19A-20C for the kidney cancer cell lines and FIG. 19D for the breast cancer cell line. All three kidney cancer cell lines were sensitive to treatment with saporin-TIM-1-specific antibody conjugates as evidenced by a measurable decrease in BrdU incorporation. Treatment of the same cell lines with conjugated irrelevant antibodies had little or no effect demonstrating antigen dependent antiproliferative effects. The same studies performed with the BT549 cell line showed that the TIM-1-specific antibody 2.56.2 showed no antiproliferative effect either alone or when conjugated to saporin. The negative controls for these studies appeared to work well with no cytotoxic effects

### Example 19

### Sequences

Below are sequences related to monoclonal antibodies against TIM-1. With regard to the amino acid sequences, bold indicates framework regions, underlining indicates CDR regions, and italics indicates constant regions.

### Anti-TIM-1 mAb 1.29

Nucleotide sequence of heavy chain variable region and a portion of constant region:

(SEQ ID NO: 1)
5′TGGGTCCTGTCCCAGGTGCAGCTGCAGGAGTCGGGCCCAGGACTGGTG
AAGCCTTCGGAGACCCTGTCCCTCACCTGCACTGTCTCTGGTGGCTCCGT
CAGCAGTGGTGGTTACTACTGGAGCTGGATCCGGCAGCCCCCAGGGAAGG
GACTGGAGTGGATTGGGTTTATCTATTACACTGGGAGCACCAACTACAAC
CCCTCCCTCAAGAGTCGAGTCTCCATATCAGTAGACACGTCCAAGAACCA
GTTCTCCCTGAAGCTGAGCTCTGTGACCGCTGCGGACGCGGCCGTGTATT
ACTGTGCGAGAGATTATGACTGGAGCTTCCACTTTGACTACTGGGGCCAG
GGAACCCTGGTCACCGTCTCCTCAGCCTCCACCAAGGGCCCATCGGTCTT
CCCCCTGGCGCCCTGCTCCAGGAGCACCTCCGAGAGCACAGCGGCCCTGG
GCTGCCTGGTCAAGGACTACTTCCCCGAACCGGTGACGGTGTCGTGGAAC
TCAGGCGCTCT3′

Amino acid sequence of heavy chain variable region and a portion of constant region encoded by SEQ ID NO:1:

(SEQ ID NO: 114)
WVLSQVQLQESGPGLVKPSETLSLTCTVSGGSVSSGGYYWSWIRQPPGKG
LEWIGFIYYTGSTNYNPSLKSRVSISVDTSKNQFSLKLSSVTAADAAVYY
CARDYDWSFHFDYWGQGTLVTVSSASTKGPSVFPLAPCSRSTSESTAALG
CLVKDYFPEPVTVSWNSGA

Nucleotide sequence of light chain variable region and a portion of constant region:

(SEQ ID NO: 3)
5′CAGCTCCTGGGGCTCCTGCTGCTCTGGTTCCCAGGTGCCAGGTGTGAC
ATCCAGATGACCCAGTCTCCATCCTCCCTGTCTGCATCTATAGGAGACAG
AGTCACCATCACTTGCCGGGCAAGTCAGGGCATTAGAAATGATTTAGGCT
GGTATCAGCAGAAACCAGGGAAAGCCCCTAAGCGCCTGATCTATGCTGCA
TCCAGTTTGCAAAGTGGGGTCCCATCAAGGTTCAGCGGCAGTGGATCTGG
GACAGAATTCACTCTCACAATCAGCAGCCTGCAGCCTGAAGATTTTGCAA
CTTATTACTGTCTACAGCATAATAGTTACCCTCTCACTTTCGGCGGAGGG
ACCAAGGTGGAGATCAAACGAACTGTGGCTGCACCATCTGTCTTCATCTT
CCCGCCATCTGATGAGCAGTTGAAATCTGGAACTGCCTCTGTTGTGTGCC
TGCTGAATAACTTCTATCCCAGAGAGGCCAAAGTACAGTGGAAGGTGGAT
AACGCC3′

Amino acid sequence of light chain variable region and a portion of constant region encoded by SEQ ID NO:3:

(SEQ ID NO: 115)
QLLGLLLLWFPGARCDIQMTQSPSSLSASIGDRVTITCRASQGIRNDLGW
YQQKPGKAPKRLIYAASSLQSGVPSRFSGSGSGTEFTLTISSLQPEDFAT
YYCLQHNSYPLTFGGGTKVEIKRTVAAPSVFIFPPSDEQLKSGTASVVCL
LNNFYPREAKVQWKVDNA

### Anti-TIM-1 mAb 1.37

Nucleotide sequence of heavy chain variable region and a portion of constant region:

(SEQ ID NO: 5)
5′CAGTGTGAGGTGCAGCTGGTGGAGTCTGGGGGAGGCTTGGTCCAGCCT
GGGGGGTCCCTGAGACTCTCCTGTGCAGCCTCTGGATTCACCTTTACTAA
CTATTGGATGAGCTGGGTCCGCCAGGCTCCAGGGAAGGGGCTGGAGTGGG
TGGCCAACATACAGCAAGATGGAAGTGAGAAATACTATGTGGACTCTGTG
AGGGGCCGATTCACCATCTCCAGAGACAACGCCAAGAACTCACTGTATCT
GCAAATGAACAGCCTGAGAGCCGAGGACTCGGCTGTGTATTACTGTGCGA
GATGGGACTACTGGGGCCAGGGAACCCTGGTCACCGTCTCCTCAGCCTCC
ACCAAGGGCCCATCGGTCTTCCCCCTGGCGCCCTGCTCCAGGAGCACCTC
CGAGAGCACAGCGGCCCTGGGCTGCCTGGTCAAGGACTACTTCCCCGAAC
CGGTGAGCGGTGTCGTGGAAC3′

Amino acid sequence of heavy chain variable region and a portion of constant region encoded by SEQ ID NO:5:

(SEQ ID NO: 116)
QCEVQLVESGGGLVQPGGSLRLSCAASGFTFTNYWMSWVRQAPGKGLEWV
ANIQQDGSEKYYVDSVRGRFTISRDNAKNSLYLQMNSLRAEDSAVYYCAR
WDYWGQGTLVTVSSASTKGPSVFPLAPCSRSTSESTAALGCLVKDYFPEP
VSGVVE

Nucleotide sequence of light chain variable region and a portion of constant region:

(SEQ ID NO: 7)
5′CTTCTGGGGCTGCTAATGCTCTGGGTCCCTGGATCCAGTGGGGATATT
GTGATGACCCAGACTCCACTCTCCTCAACTGTCATCCTTGGACAGCCGGC
CTCCATCTCCTGCAGGTCTAGTCAAAGCCTCGTACACAGTGATGGAAACA
CCTACTTGAATTGGCTTCAGCAGAGGCCAGGCCAGCCTCCAAGACTCCTA
ATTTATATGATTTCTAACCGGTTCTCTGGGGTCCCAGACAGATTCAGTGG
CAGTGGGGCAGGGACAGATTTCACACTGAAAATCAGCAGGGTGGAAGCTG
AGGATGTCGGGGTTTATTACTGCATGCAAGCTACAGAATCTCCTCAGACG
TTCGGCCAAGGGACCAAGGTGGAAATCAAACGAACTGTGGCTGCACCATC
TGTCTTCATCTTCCCGCCATCTGATGAGCAGTTGAAATCTGGAAGGGCCT
CTGTTG3′

Amino acid sequence of light chain variable region and a portion of constant region encoded by SEQ ID NO:7:

(SEQ ID NO: 117)
LLGLLMLWVPGSSGDIVMTQTPLSSTVILGQPASISCRSSQSLVHSDGNT
YLNWLQQRPGQPPRLLIYMISNRFSGVPDRFSGSGAGTDFTLKISRVEAE
DVGVYYCMQATESPQTFGQGTKVEIKRTVAAPSVFIFPPSDEQLKSGRAS
V

### Anti-TIM-1 mAb 2.16

Nucleotide sequence of heavy chain variable region and a portion of constant:

(SEQ ID NO: 9)
5′GAGCAGTCGGGGGGAGGCGTGGTAAAGCCTGGGGGGTCTCTTAGACTC
TCCTGTGCAGCCTCTGGATTCACTTTCAGTAACGCCTGGATGACCTGGGT
CCGCCAGGCTCCAGGGAAGGGGCTGGAGTGGGTTGGCCGTATTAAAAGGA
GAACTGATGGTGGGACAACAGACTACGCTGCACCCGTGAAAGGCAGATTC
ACCATCTCAAGAGATGATTCAAAAAACACGCTGTATCTGCAAATGAACAA
CCTGAAAAACGAGGACACAGCCGTGTATTACTGTACCTCAGTCGATAATG
ACGTGGACTACTGGGGCCAGGGAACCCTGGTCACCGTCTCCTCAGCTTCC
ACCAAGGGCCCATCCGTCTTCCCCCTGGCGCCCTGCTCCAGGAGCACCTC
CGAGAGCACAGCCGCCCTGGGCTGCCTGGTCAAGGACTACTTCCCCGAAC
CGGTGACGGTGTCGTGGAACTCAGGCGCCCTGACCAGCGGCGTGCACACC
TTCCCGGCTGTCCTACAGTCCTCAGGACTCT3′

Amino acid sequence of heavy chain variable region and a portion of constant region encoded by SEQ ID NO:9:

(SEQ ID NO: 118)
XXXXEQSGGGVVKPGGSLRLSCAASGFTFSNAWMTWVRQAPGKGLEWVGR
IKRRTDGGTTDYAAPVKGRFTISRDDSKNTLYLQMNNLKNEDTAVYYCTS
VDNDVDYWGQGTLVTVSSASTKGPSVFPLAPCSRSTSESTAALGCLVKDY
FPEPVTVSWNSGALTSGVHTFPAVLQSSGL

Nucleotide sequence of light chain variable region and a portion of constant region:

(SEQ ID NO: 11)
5′CTGACTCAGTCTCCACTCTCCCTGCCCGTCACCCCTGGAGAGCCGGCC
TCCATCTCCTGCAGGTCTAGTCAGAGCCTCCTGCATAGTAATGGATACAA
CTATTTGGATTGGTACCTGCAGAAGCCAGGGCAGTCTCCACAGCTCCTGA
TCTATTTGGGTTCTAATCGGGCCTCCGGGGTCCCTGACAGGTTCAGTGGC
AGTGGATCAGGCACAGATTTTACACTGAAAATCAGCAGAGTGGAGGCTGA
GGATATTGGTCTTTATTACTGCATGCAAGCTCTACAAACTCCGCTCACTT
TCGGCGGAGGGACCAAGGTGGACATCAAACGAACTGTGGCTGCACCATCT
GTCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
3′

Amino acid sequence of light chain variable region and a portion of constant region encoded by SEQ ID NO:11:

(SEQ ID NO: 119)
XXXLTQSPLSLPVTPGEPASISCRSSQSLLHSNGYNYLDWYLQKPGQSPQ
LLIYLGSNRASGVPDRFSGSGSGTDFTLKISRVEAEDIGLYYCMQALQTP
LTFGGGTKVDIKRTVAAPSVFIFPPSDEQLKSGTASVVCLLNNFYPREAK
VQ

### Anti-TIM-1 mAb 2.17

Nucleotide sequence of heavy chain variable region and a portion of constant region:

(SEQ ID NO: 13)
5′CAGGTGCAGCTGGAGCAGTCGGGGGGAGGCTTGGTACAGCCTGGGGGG
TCCCTGAGACTCTCCTGTGCAGCCTCTGGATTCACCTTCAGTACCTATAG
CATGAACTGGGTCCGCCAGGCTCCAGGGAAGGGGCTGGAGTGGGTTTCAT
ACATTAGAAGTAGTACTAGTACCATATACTATGCAGAGTCCCTGAAGGGC
CGATTCACCATCTCCAGCGACAATGCCAAGAATTCACTATATCTGCAAAT
GAACAGCCTGAGAGACGAGGACACGGCTGTGTATTACTGTGCGCGGGACT
TTGACTACTGGGGCCAGGGAACCCTGGTCACCGTCTCCTCAGCTTCCACC
AAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTC
CCGGCTGTCCTACAGTCCTCAGGACTCTACTCCCTCAGCA3′

Amino acid sequence of heavy chain variable region and a portion of constant region encoded by SEQ ID NO:13:

(SEQ ID NO: 120)
QVQLEQSGGGLVQPGGSLRLSCAASGFTFSTYSMNWVRQAPGKGLEWVSY
IRSSTSTIYYAESLKGRFTISSDNAKNSLYLQMNSLRDEDTAVYYCARDF
DYWGQGTLVTVSSASTKGPSVFPLAPCSRSTSESTAALGCLVKDYFPEPV
TVSWNSGALTSGVHTFPAVLQSSGLYSLS

Nucleotide sequence of light chain variable region and a portion of constant region:

(SEQ ID NO: 15)
5′GAAATCCAGCTGACTCAGTCTCCACTCTCCTCACCTGTCACCCTTGGA
CAGCCGGCCTCCATCTCCTGCAGGTCTAGTCAAAGCCTCGTACACAGTGA
TGGAGACACCTACTTGAATTGGCTTCAGCAGAGGCCAGGCCAGCCTCCAA
GACTCCTAATTTATAAGATTTCTACCCGGTTCTCTGGGGTCCCTGACAGA
TTCAGTGGCAGTGGGGCAGGGACAGATTTCACACTGAAAATCAGCAGGGT
GGAGACTGACGATGTCGGGATTTATTACTGCATGCAAACTACACAAATTC
CTCAAATCACCTTCGGCCAAGGGACACGACTGGAGATTAAACGAACTGTG
GCTGCACCATCTGTCTTCATCTTCCCGCCATCTGATGAGCAGTTGAAATC
TGGAACTGCCTCTGTTGTGTGCCTGCTGAATAACTTCTATCCCAGAGAGG
CCAAAGTACAGTGGAAGGTGGATAACGCCCTCCAATCGGGTA3′

Amino acid sequence of light chain variable region and a portion of constant region encoded by SEQ ID NO:15:

(SEQ ID NO: 121)
EIQLTQSPLSSPVTLGQPASISCRSSQSLVHSDGDTYLNWLQQRPGQPPR
LLIYKISTRFSGVPDRFSGSGAGTDFTLKISRVETDDVGIYYCMQTTQIP
QITFGQGTRLEIKRTVAAPSVFIFPPSDEQLKSGTASVVCLLNNFYPREA
KVQWKVDNALQSG

### Anti-TIM-1 mAb 2.24

Nucleotide sequence of heavy chain variable region and a portion of constant region:

(SEQ ID NO: 17)
5′CAGGTGCAGCTGGAGCAGTCGGGGGGAGGCGTGGTCCAGCCTGGGAGG
TCCCTGAGACTCTCCTGTGCAGCGTCTGGATTCACCTTCAGTCGCTATGG
CATGCACTGGGTCCGCCAGGCTCCAGGCAAGGGGCTGAAATGGGTGGCAG
TTATATGGTATGATGGAAGTAATAAACTCTATGCAGACTCCGTGAAGGGC
CGATTCACCATCTCCAGAGACAATTCCAAGAACACGCTGTATCTGCAAAT
GAACAGCCTGAGAGCCGAGGACACGGCTGTGTATTACTGTGCGAGAGATT
ACTATGATAATAGTAGACATCACTGGGGGTTTGACTACTGGGGCCAGGGA
ACCCTGGTCACCGTCTCCTCAGCTTCCACCAAGGGCCCATCCGTCTTCCC
CCTGGCGCCCTGCTCCAGGAGCACCTCCGAGAGCACAGCCGCCCTGGGCT
GCCTGGTCAAGGACTACTTCCCCGAACCGGTGACGGTGTCGTGGAACTCA
GGCGCCCTGACCAGCGGCGTGCACACCTTCCCGGCTGTCCTACAGTCCTC
AGGACTCTACTCCCTCAGCA

Amino acid sequence of heavy chain variable region and a portion of constant region encoded by SEQ ID NO:17:

(SEQ ID NO: 122)
QVQLEQSGGGVVQPGRSLRLSCAASGFTFSRYGMHWVRQAPGKGLKWVAV
IWYDGSNKLYADSVKGRFTISRDNSKNTLYLQMNSLRAEDTAVYYCARDY
YDNSRHHWGFDYWGQGTLVTVSSASTKGPSVFPLAPCSRSTSESTAALGC
LVKDYFPEPVTVSWNSGALTSGVHTFPAVLQSSGLYSLS

Nucleotide sequence of light chain variable region and a portion of constant region:

(SEQ ID NO: 19)
5′GACATCCAGCTGACCCAGTCTCCATCCTCCCTGTCTGCATCTGTAGGA
GACAGAGTCACCATCACTTGCCGGGCAAGTCAGAGTATTTATAGTTATTT
AAATTGGTATCAGCAGAAACCAGGGAAAGCCCCTAAGCTCCTGATCTATG
CTGCATCCAGTTTGCAAAGTGGGGTCCCATCCAGGTTCAGTGGCAGTGGA
TCTGGGACAGATTTCACTCTCACCATCAGCAGTCTGCAACCTGAAGATTT
TGCAACTTACTACTGTCAACAGAGTTACAGTACCCCTCCGACGTTCGGCC
AAGGGACCAAGGTGGAAATCAAACGAACTGTGGCTGCACCATCTGTCTTC
ATCTTCCCGCCATCTGATGAGCAGTTGAAATCTGGAACTGCCTCTGTTGT
GTGCCTGCTGAATAACTTCTATCCCAGAGAGGCCAAAGTACAGTGGAAGG
TGGATAACGCCCTCCAATCGGGTA3′

Amino acid sequence of light chain variable region and a portion of constant region encoded by SEQ ID NO:19:

(SEQ ID NO: 123)
DIQL/MT/LQSPSSLSASVGDRVTITCRASQSIYSYLNWYQQKPGKAPKL
LIYAASSLQSGVPSRFSGSGSGTDFTLTISSLQPEDFATYYCQQSYSTPP
TFGQGTKVEIKRTVAAPSVFIFPPSDEQLKSGTASVVCLLNNFYPREAKV
QWKVDNALQSG

### Anti-TIM-1 mAb 2.45

Nucleotide sequence of heavy chain variable region and a portion of constant region:

(SEQ ID NO: 21)
5′CAGTCGGGGGGAGGCTTGGTAAAGCCTGGGGGGTCCCTTAGACTCTCC
TGTGCAGCCTCTGGATTCACTTTCAGTAACGCCTGGATGACCTGGGTCCG
CCAGGCTCCAGGGAAGGGGCTGGAGTGGGTTGGCCGTATTAAAAGGAAAA
CTGATGGTGGGACAACAGACTACGCTGCACCCGTGAAAGGCAGATTCACC
ATCTCAAGAGATGATTCAGAAAACACGCTGTATCTGCAAATGAACAGCCT
GGAAACCGAGGACACAGCCGTGTATTACTGTACCACAGTCGATAACAGTG
GTGACTACTGGGGCCAGGGAACCCTGGTCACCGTCTCCTCAGCTTCCACC
AAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTC
CCGGCTGTCCTACAGTCCTCAGGACTCTCT3′

Amino acid sequence of heavy chain variable region and a portion of constant region encoded by SEQ ID NO:21:

(SEQ ID NO: 124)
XXXXXQSGGGLVKPGGSLRLSCAASGFTFSNAWMTWVRQAPGKGLEWVGR
IKRKTDGGTTDYAAPVKGRFTISRDDSENTLYLQMNSLETEDTAVYYCTT
VDNSGDYWGQGTLVTVSSASTKGPSVFPLAPCSRSTSESTAALGCLVKDY
FPEPVTVSWNSGALTSGVHTFPAVLQSSGLS

Nucleotide sequence of light chain variable region and a portion of constant region:

(SEQ ID NO: 23)
5′ACTCAGTCTCCACTCTCCCTGCCCGTCACCCCTGGAGAGCCGGCCTCC
ATCTCCTGCAGGTCTAGTCAGAGCCTCCTGCATAGTAATGGATACAACTA
TTTGGATTGGTACCTGCAGAAGCCAGGGCAGTCTCCACAGCTCCTGATCT
ATTTGGGTTCTAATCGGGCCTCCGGGGTCCCTGACAGGTTCAGTGGCAGT
GGATCAGGCACAGATTTTACACTGAAAATCAGCAGAGTGGAGGCTGAGGA
TGTTGGGGTTTATTACTGCATGCAAGCTCTACAAACTCCGCTCACTTTCG
GCGGAGGGACCAAGGTGGAGATCAAACGAACTGTGGCTGCACCATCTGTC
TTCATCTTCCCGCCATCTGATGAGCAGTTGAAATCTGGAACTGCCTCTGT
TGTGTGCCTGCTGAATAACTTCTATCCCAGAGAGGCCAAAGTACAGTGGA
AGGTGGATAACGCCCTCA3′

Amino acid sequence of light chain variable region and a portion of constant region encoded by SEQ ID NO:23:

(SEQ ID NO: 125)
XXXXTQSPLSLPVTPGEPASISCRSSQSLLHSNGYNYLDWYLQKPGQSPQ
LLIYLGSNRASGVPDRFSGSGSGTDFTLKISRVEAEDVGVYYCMQALQTP
LTFGGGTKVEIKRTVAAPSVFIFPPSDEQLKSGTASVVCLLNNFYPREAK
VQWKVDNAL

### Anti-TIM-1 mAb 2.54

Nucleotide sequence of heavy chain variable region and a portion of constant region:

(SEQ ID NO: 25)
5′CAGGTGCAGCTGGAGCAGTCGGGGGGAGGCGTGGTCCAGCCTGGGAGG
TCCCTGAGACTCTCCTGTGCAGCGTCTGGATTCACCTTCACTAACTATGG
CTTGCACTGGGTCCGCCAGGCTCCAGGCAAGGGGCTGGATTGGGTGGCAG
TTATATGGTATGATGGAAGTCATAAATTCTATGCAGACTCCGTGAAGGGC
CGATTCACCATCTCCAGAGACAATTCCAAGAACACGCTCTTTCTGCAAAT
GAACAGCCTGAGAGCCGAGGACACGGCTGTGTATTACTGTACGCGAGATC
TTGACTACTGGGGCCAGGGAACCCTGGTCACCGTCTCCTCAGCTTCCACC
AAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGG
TGACGGTGTCGTGGAACTCAGGCGCCCTGACCAGCGGCGTGCACACCTTC
CCGGCTGTCCTACAGTCCTCAGGACTCTACTCCCTCAGC3′

Amino acid sequence of heavy chain variable region and a portion of constant region encoded by SEQ ID NO:25:

(SEQ ID NO: 126)
QVQLEQSGGGVVQPGRSLRLSCAASGFTFTNYGLHWVRQAPGKGLDWVAVI
WYDGSHKFYADSVKGRFTISRDNSKNTLFLQMNSLRAEDTAVYYCTRDLDY
WGQGTLVTVSSASTKGPSVFPLAPCSRSTSESTAALGCLVKDYFPEPVTVS
WNSGALTSGVHTFPAVLQSSGLYSLS

Nucleotide sequence of light chain variable region and a portion of constant region:

(SEQ ID NO: 27)
5′GAAACGCAGCTGACGCAGTCTCCAGGCACCCTGTCTTTGTCTCCAGGG
GAAAGAGTCACCCTCTCCTGCAGGGCCAGTCAGAGTGTTAGCAACAACTA
CTTAGCCTGGTACCAGCAGAAACCTGGCCAGGCTCCCAGGCTCCTCATCT
ATGGTGCATCCAGCAGGGCCACTGGCATCCCAGACAGGTTCAGTGGCAGT
GGGTCTGGGACAGACTTCACTCTCACCATCAGCAGACTGGAGCCTGAAGA
TTGTGCAGAGTGTTACTGTCAGCAATATGGTAGCTCACTCCCGCTCACTT
TCGGCGGAGGGACCAAGGTGGAGATCAAACGAACTGTGGCTGCACCATCT
GTCTTCATCTTCCCGCCATCTGATGAGCAGTTGAAATCTGGAACTGCCTC
TGTTGTGTGCCTGCTGAATAACTTCTATCCCAGAGAGGCCAAAGTACAGT
GGGAAGGTGGGATAACGCCCTCCAATCGGGTA3′

Amino acid sequence of light chain variable region and a portion of constant region encoded by SEQ ID NO:27:

(SEQ ID NO: 127)
ETQLTQSPGTLSLSPGERVTLSCRASQSVSNNYLAWYQQKPGQAPRLLIY
GASSRATGIPDRFSGSGSGTDFTLTISRLEPEDCAECYCQQYGSSLPLTF
GGGTKVEIKRTVAAPSVFIFPPSDEQLKSGTASVVCLLNNFYPREAKVQW
EGGITPSNRV

### Anti-TIM-1 mAb 2.56

Nucleotide sequence of heavy chain variable region and a portion of constant region:

(SEQ ID NO: 29)
5′GTCCAGTGTCAGGTGCAGCTGGTGGAGTCTGGGGGAGGCGTGGTCCAG
CCTGGGAGGTCCCTGAGACTCTCCTGTGCAGCGTCTGGATTCACCTTCAG
TAGCTATGGCATGCACTGGGTCCGCCAGGCTCCAGGCAAGGGGCTGGAGT
GGGTGGCAGTTATATGGTATGATGGAAGTCATAAATACTATGCAGACTCC
GTGAAGGGCCGATTCACCATCTCCAGAGACAATTCCAAGAACACGCTGTA
TCTGCAAATGAACAGCCTGAGAGCCGAGGACACGGCTGTGTATTACTCTG
CGAGAGATTACTATGATACGAGTCGGCATCACTGGGGGTTTGACTGCTGG
GGCCAGGGAACCCTGGTCACCGTCTCCTCTGCTTCCACCAAGGGCCCATC
CGTCTTCCCCCTGGCGCCCTGCTCCAGGAGCACCTCCGAGAGCACAGCCG
CCCTGGGCTGCCTGGTCAAGGACTACTTCCCCGAACCGGTGACGGTGTCG
TGGAACTCAGGCGCCCTGACCAGCGGCGTGCACACCTTCCCGGC3′

Amino acid sequence of heavy chain variable region and a portion of constant region encoded by SEQ ID NO: 29:

(SEQ ID NO: 128)
VQCQVQLVESGGGVVQPGRSLRLSCAASGFTFSSYGMHWVRQAPGKGLEW
VAVIWYDGSHKY/LYA/TDSVKGRFTISRDNSKNTLYLQMNSLRAEDTAV
YYSARDYYDTSRHHWGFDCWGQGTLVTVSSASTKGPSVFPLAPCSRSTSE
STAALGCLVKDYFPEPVTVSWNSGALTSGVHTFP

Nucleotide sequence of light chain variable region and a portion of constant region:

(SEQ ID NO: 31)
5′CAGCTCCTGGGGCTGCTAATGCTCTGGGTCCCTGGATCCAGTGAGGAA
ATTGTGATGACCCAGACTCCACTCTCCCTGCCCGTCACCCCTGGAGAGCC
GGCCTCCATCTCCTGCAGGTCTAGTCAGAGCCTCTTGGATAGTGAAGATG
GAAACACCTATTTGGACTGGTACCTGCAGAAGCCAGGGCAGTCTCCACAG
CTCCTGATCTATACGCTTTCCCATCGGGCCTCTGGAGTCCCAGACAGGTT
CAGTGGCAGTGGGTCAGGCACTGATTTCACACTGAAAATCAGCAGGGTGG
AGGCTGAGGATGTTGGAGTTTATTGCTGCATGCAACGTGTAGAGTTTCCT
ATCACCTTCGGCCAAGGGACACGACTGGAGATTAAACGAACTGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGC3′

Amino acid sequence of light chain variable region and a portion of constant region encoded by SEQ ID NO:31:

(SEQ ID NO: 129)
QLLGLLMLWVPGSSEEIVMTQTPLSLPVTPGEPASISCRSSQSLLDSEDGN
TYLDWYLQKPGQSPQLLIYTLSHRASGVPDRFSGSGSGTDFTLKISRVEAE
DVGVYCCMQRVEFPITFGQGTRLEIKRTVAAPSVFIFPPSDEQLKSGTASV
VCLLNNFYPREAKVQWKVDN

### Anti-TIM-1 mAb 2.59

Nucleotide sequence of heavy chain variable region and a portion of constant region:

(SEQ ID NO: 33)
5′CAGTCGGGCCCAAGACTGGTGAAGCCTTCACAGACCCTGTCCCTCACC
TGCACTGTCTCTGGTGGCTCCATCAGTAGTGATGGTTACTACTGGAGCTG
GATCCGCCAGCACCCAGGGAAGGGCCTGGAGTGGATTGGGTACATCTATT
ACAGTGGGAGCACCTTCTACAACCCGTCCCTCAAGAGTCGAGTTGCCATA
TCAGTGGACACGTCTAAGAACCAGTTCTCCCTGAAGCTGAGCTCTGTGAC
TGCCGCGGACACGGCCGTGTATTACTGTGCGAGAGAATCCCCTCATAGCA
GCAACTGGTACTCGGGCTTTGACTGCTGGGGCCAGGGAACCCTGGTCACC
GTCTCCTCAGCTTCCACCAAGGGCCCATCCGTCTTCCCCCTGGCGCCCTG
CTCCAGGAGCACCTCCGAGAGCACAGCCGCCCTGGGCTGCCTGGTCAAGG
ACTACTTTCCCCGAACCGGTGACGGTGTCGTGGAACTCAGGCGCCCTGAC
CAGCGGCGTGCACACCTTCCCGGCTGTCCTACAGTCCTCAGGACTCTCT3′

Amino acid sequence of heavy chain variable region and a portion of constant region encoded by SEQ ID NO:33:

(SEQ ID NO: 130)
XXXXXQSGPRLVKPSQTLSLTCTVSGGSISSDGYYWSWIRQHPGKGLEWI
GYIYYSGSTFYNPSLKSRVAISVDTSKNQFSLKLSSVTAADTAVYYCARE
SPHSSNWYSGFDCWGQGTLVTVSSASTKGPSVFPLAPCSRSTSESTAALG
CLVKDYFPRTGDGVVELRRPDQRRAHLPGCPTVLRTL

Nucleotide sequence of light chain variable region and a portion of constant region:

(SEQ ID NO: 35)
5′ACTCAGTCTCCAGACTTTCAGTCTGTGACTCCAAAGGAGAAAGTCACCA
TCACCTGCCGGGCCAGTCAGAGCATTGGTAGTAGGTTACACTGGTACCAGC
AGAAACCAGATCAGTCTCCAAAGCTCCTCATCAAGTATGCTTCCCAGTCCT
TCTCAGGGGTCCCCTCGAGGTTCAGTGGCAGTGGATCTGGGACAGATTTCA
CCCTCACCATCAATAGCCTGGAAGCTGAAGATGCTGCAACGTATTACTGTC
ATCAGAGTAGTAATTTACCATTCACTTTCGGCCCTGGGACCAAAGTGGATA
TCAAACGAACTGTGGCTGCACCATCTGTCTTCATCTTCCCGCCATCTGATG
AGCAGTTGAAATCTGGAACTGCCTCTGTTGTGTGCCTGCTGAATAACTTCT
ATCCCAGAGAGGCCAAAGTACAGTGGAAGGTGGATAACGCCCTC3′

Amino acid sequence of light chain variable region and a portion of constant region encoded by SEQ ID NO:35:

(SEQ ID NO: 131)
XXXXTQSPDFQSVTPKEKVTITCRASQSIGSRLHWYQQKPDQSPKLLIKY
ASQSFSGVPSRFSGSGSGTDFTLTINSLEAEDAATYYCHQSSNLPFTFGP
GTKVDIKRTVAAPSVFIFPPSDEQLKSGTASVVCLLNNFYPREAKVQWKV
DNAL

### Anti-TIM-1 mAb 2.61

Nucleotide sequence of heavy chain variable region and a portion of constant region:

(SEQ ID NO: 37)
5′CAGGTGCAGCTGGTGGAGGCTGGGGGAGGCGTGGTCCAGCCTGGGAGG
TCCCTGAGACTCTCCTGTGCAGCGTCTGGATTCACCTTCAGAAGCTATGG
CATGCACTGGGTCCGCCAGGCTCCAGGCAAGGGGCTGAAATGGGTGGCAG
TTATATGGTATGATGGAAGTAATAAATACTATACAGACTCCGTGAAGGGC
CGATTCACCATCTCCAGAGACAATTCCAAGAACACGCTGTATCTGCAAAT
GAACAGCCTGAGAGCCGAGGACACGGCTGTGTATTACTGTGTGAGAGATT
ACTATGATAATAGTAGACATCACTGGGGGTTTGACTACTGGGGCCAGGGA
ACCCTGGTCACCGTCTCCTCAGCTTCCACCAAGGGCCCATCCGTCTTCCC
CCTGGCGCCCTGCTCCAGGAGCACCTCCGAGAGCACAGCCGCCCTGGGCT
GCCTGGTCAAGGACTACTTCCCCGAACCGGTGACGGTGTCGTGGAACTCA
GGCGCCCTGACCAGGCGGCGTGCACACCTTCCCGGC3′

Amino acid sequence of heavy chain variable region and a portion of constant region encoded by SEQ ID NO:37:

(SEQ ID NO: 132)
QVQLVE/QAGGGVVQPGRSLRLSCAASGFTFRSYGMHWVRQAPGKGLKWV
AVIWYDGSNKY/LYTDSVKGRFTISRDNSKNTLYLQMNSLRAEDTAVYYC
VRDYYDNSRHHWGFDYWGQGTLVTVSSASTKGPSVFPLAPCSRSTSESTA
ALGCLVKDYFPEPVTVSWNSGALTRRRAHLPG

Nucleotide sequence of light chain variable region and a portion of constant region:

(SEQ ID NO: 39)
5′GACATCCAGATGACCCAGTCTCCATCCTCCCGGTGTGCATCCGTAGGA
GACAGAGTCACCATCACTTGCCGGGCAAGTCAGGGCATCAGAAATGATTT
AGCTTGGTATCAGCAGAAACCAGGGAAAGCCCCTAAGCGCCTGATCTATG
CTGCATCCAGTTTGCAAAGTGGGGTCCCATCAAGGTTCAGCGGCAGTAGA
TCTGGGACAGAATTCACTCTCACAATCAGCAGCCTGCAGCCTGAAGATTT
TGCAGCTTATTACTGTCTCCAGCATAATAGTTACCCTCCCAGTTTTGGCC
AGGGGACCAAGCTGGAGATCAAACGAACTGTGGCTGCACCATCTGTCTTC
ATCTTCCCGCCATCTGATGAGCAGTTGAAATCTGGAACTGCTAGCGTTGT
GTGCCTGCTGAATAACTTCTATCCCAGAGAGGCCAAAGTACAGTGGAAGG
TGGATAACGCCCTCCAATCGGG3′

Amino acid sequence of light chain variable region and a portion of constant region encoded by SEQ ID NO:39:

(SEQ ID NO: 133)
DIQMTQSPSSRCASVGDRVTITCRASQGIRNDLAWYQQKPGKAPKRLIYA
ASSLQSGVPSRFSGSRSGTEFTLTISSLQPEDFAAYYCLQHNSYPPSFGQ
GTKLEIKRTVAAPSVFIFPPSDEQLKSGTASVVCLLNNFYPREAKVQWKV
DNALQS

### Anti-TIM-1 mAb 2.70

Nucleotide sequence of heavy chain variable region and a portion of constant region:

(SEQ ID NO: 41)
5′CATGTGCAGGTGCAGCTGGTGGAGTCTGGGGGAGGCGTGGTCCAGCCT
GGGAGGTCCCTGAGACTCTCCTGTGCAGCGTCTGGATTCATCTTCAGTCG
CTATGGCATGCACTGGGTCCGCCAGGCTCCAGGCAAGGGGCTGAAATGGG
TGGCAGTTATATGGTATGATGGAAGTAATAAACTCTATGCAGACTCCGTG
AAGGGCCGATTCACCATCTCCAGAGACAATTCCAAGAACACGCTGTATCT
GCAAATGAACAGCCTGAGAGCCGAGGACACGGCTGTGTATTACTGTGCGA
GAGATTACTATGATAATAGTAGACATCACTGGGGGTTTGACTACTGGGGC
CAGGGAACCCTGGTCACCGTCTCCTCAGCTTCCACCAAGGGCCCATCCGT
CTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGG
AACTCAGGCGCCCTGA3′

Amino acid sequence of heavy chain variable region and a portion of constant region encoded by SEQ ID NO:41:

(SEQ ID NO: 134)
HVQVQLVESGGGVVQPGRSLRLSCAASGFIFSRYGMHWVRQAPGKGLKWV
AVIWYDGSNKLYADSVKGRFTISRDNSKNTLYLQMNSLRAEDTAVYYCAR
DYYDNSRHHWGFDYWGQGTLVTVSSASTKGPSVFPLAPCSRSTSESTAAL
GCLVKDYFPEPVTVSWNSGAL

Nucleotide sequence of light chain variable region and a portion of constant region:

(SEQ ID NO: 43)
5′TCAGCTCCTGGGGCTGCTAATGCTCTGGGTCCCTGGATCAGTGAGGAT
ATTGTGATGACCCAGACTCCACTCTCCCTGCCCGTCACCCCTGGAGAGCC
GGCCTCCATCTCCTGCAGGTCTAGTCGGAGCCTCTTGGATAGTGATGATG
GAAACACCTATTTGGACTGGTACCTGCAGAAGCCAGGGCAGTCTCCACAG
CTCCTGATCTACACGCTTTCCTATCGGGCCTCTGGAGTCCCAGACAGGTT
CAGTGGCAGTGGGTCAGGCACTGATTTCACACTGAAAATCAGCAGGGTGG
AGGCTGAGGATGTTGGAGTTTATTACTGCATGCAACGTGTAGAGTTTCCT
ATCACCTTCGGCCAAGGGACACGACTGGAGATTAAACGAACTGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCT3′

Amino acid sequence of light chain variable region and a portion of constant region encoded by SEQ ID NO:43:

(SEQ ID NO: 135)
SAPGAANALGPWISEDIVMTQTPLSLPVTPGEPASISCRSSRSLLDSDDG
NTYLDWYLQKPGQSPQLLIYTLSYRASGVPDRFSGSGSGTDFTLKISRVE
AEDVGVYYCMQRVEFPITFGQGTRLEIKRTVAAPSVFIFPPSDEQLKSGT
ASVVCLLNNFYPREAKVQWKVDNA

### Anti-TIM-1 mAb 2.70.2

Nucleotide sequence of heavy chain variable region and a portion of constant region:

(SEQ ID NO: 136)
5′CGGCCGCCTATTTACCCAGAGACAGGGAGAGGCTCTTCTGTGTGTAGT
GGTTGTGCAGAGCCTCATGCATCACGGAGCATGAGAAGACATTCCCCTCC
TGCCACCTGCTCTTGTCCACGGTTAGCCTGCTGTAGAGGAAGAAGGAGCC
GTCGGAGTCCAGCACGGGAGGCGTGGTCTTGTAGTTGTTCTCCGGCTGCC
CATTGCTCTCCCACTCCACGGCGATGTCGCTGGGGTAGAAGCCTTTGACC
AGGCAGGTCAGGCTGACCTGGTTCTTGGTCATCTCCTCCTGGGATGGGGG
CAGGGTGTACACCTGTGGCTCTCGGGGCTGCCCTTTGGCTTTGGAGATGG
TTTTCTCGATGGAGGACGGGAGGCCTTTGTTGGAGACCTTGCACTTGTAC
TCCTTGCCGTTCAGCCAGTCCTGGTGCAGGACGGTGAGGACGCTGACCAC
ACGGTACGTGCTGTTGAACTGCTCCTCCCGCGGCTTTGTCTTGGCATTAT
GCACCTCCACGCCATCCACGTACCAGTTGAACTGGACCTCGGGGTCTTCC
TGGCTCACGTCCACCACCACGCACGTGACCTCAGGGGTCCGGGAGATCAT
GAGAGTGTCCTTGGGTTTTGGGGGGAACAGGAAGACTGATGGTCCCCCCA
GGAACTCAGGTGCTGGGCATGATGGGCATGGGGGACCATATTTGGACTCA
ACTCTCTTGTCCACCTTGGTGTTGCTGGGCTTGTGATCTACGTTGCAGGT
GTAGGTCTTCGTGCCCAAGCTGCTGGAGGGCACGGTCACCACGCTGCTGA
GGGAGTAGAGTCCTGAGGACTGTAGGACAGCCGGGAAGGTGTGCACGCCG
CTGGTCAGGGCGCCTGAGTTCCACGACACCGTCACCGGTTCGGGGAAGTA
GTCCTTGACCAGGCAGCCCAGGGCGGCTGTGCTCTCGGAGGTGCTCCTGG
AGCAGGGCGCCAGGGGGAAGACGGATGGGCCCTTGGTGGAAGCTGAGGAG
ACGGTGACCAGGGTTCCCTGGCCCCAGTAGTCAAACCCCCAGTGATGTCT
ACTATTATCATAGTAATCTCTCGCACAGTAATACACAGCCGTGTCCTCGG
CTCTCAGGCTGTTCATTTGCAGATACAGCGTGTTCTTGGAATTGTCTCTG
GAGATGGTGAATCGGCCCTTCACGGAGTCTGCATAGAGTTTATTACTTCC
ATCATACCATATAACTGCCACCCATTTCAGCCCCTTGCCTGGAGCCTGGC
GGACCCAGTGCATGCCATAGCGACTGAAGATGAATCCAGACGCTGCACAG
GAGAGTCTCAGGGACCTCCCAGGCTGGACCACGCCTCCCCCAGACTCCAC
CAGCTGCACCTGACACTGGACACCTTTTAAAATAGCCACAAGAAAAAGCC
AGCTCAGCCCAAACTCCATGGTGGTCGACT3′

Amino acid sequence of heavy chain variable region and a portion of constant region encoded by SEQ ID NO:136:

(SEQ ID NO: 137)
MEFGLSWLFLVAILKGVQCQVQLVESGGGVVQPGRSLRLSCAASGFIFSR
YGMHWVRQAPGKGLKWVAVIWYDGSNKLYADSVKGRFTISRDNSKNTLYL
QMNSLRAEDTAVYYCARDYYDNSRHHWGFDYWGQGTLVTVSSASTKGPSV
FPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPS
CPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVM
HEALHNHYTQKSLSLSLGK

Nucleotide sequence of light chain variable region and a portion of constant region:

(SEQ ID NO: 138)
5′AGTCGACCACCATGGAAACCCCAGCGCAGCTTCTCTTCCTCCTGCTAC
TCTGGCTCCCAGATACCACCGGAGATATTGTGATGACCCAGACTCCACTC
TCCCTGCCCGTCACCCCTGGAGAGCCGGCCTCCATCTCCTGCAGGTCTAG
TCGGAGCCTCTTGGATAGTGATGATGGAAACACCTATTTGGACTGGTACC
TGCAGAAGCCAGGGCAGTCTCCACAGCTCCTGATCTACACGCTTTCCTAT
CGGGCCTCTGGAGTCCCAGACAGGTTCAGTGGCAGTGGGTCAGGCACTGA
TTTCACACTGAAAATCAGCAGGGTGGAGGCTGAGGATGTTGGAGTTTATT
ACTGCATGCAACGTGTAGAGTTTCCTATCACCTTCGGCCAAGGGACACGA
CTGGAGATTAAACGAACTGTGGCTGCACCATCTGTCTTCATCTTCCCGCC
ATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGA
CAGCACCTACAGCCTCAGCAGCACCCTGACGCTGAGCAAAGCAGACTACG
AGAAACACAAAGTCTACGCCTGCGAAGTCACCCATCAGGGCCTGAGCTCG
CCCGTCACAAAGAGCTTCAACAGGGGAGAGTGTTAGGCGGCCG3′

Amino acid sequence of light chain variable region and portion constant region by SEQ ID NO:138:

(SEQ ID NO: 139)
METPAQLLFLLLLWLPDTTGDIVMTQTPLSLPVTPGEPASISCRSSRSLL
DSDDGNTYLDWYLQKPGQSPQLLIYTLSYRASGVPDRFSGSGSGTDFTLK
ISRVEAEDVGVYYCMQRVEFPITFGQGTRLEIKRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGEC

### Anti-TIM-1 mAb 2.76

Nucleotide sequence of heavy chain variable region and a portion of constant region:

(SEQ ID NO: 45)
5′GAGCAGTCGGGGGGCGGCGTGGTCCAGCCTGGGAGGTCCCTGAGACTC
TCCTGTGCAGCGTCTGGATTCACCTTCAGTAGCTATGGCATGTACTGGGT
CCGCCAGGCTCCAGGCAAGGGGCTGGAGTGGGTGGCAGTTATATGGTATG
ATGGAAGCAATAAATACTATGCAGACTCCGTGAAGGGCCGATTCACCATC
TCCAGAGACAATTCCAAGAACACGCTGTATCTGCAAATGAACAGCCTGAG
AGCCGAGGACACGGCTGTGTATTACTGTGCGAGGGATTTCTATGATAGTA
GTCGTTACCACTACGGTATGGACGTCTGGGGCCAAGGGACCACGGTCACC
GTCTCCTCAGCTTCCACCAAGGGCCCATCCGTCTTCCCCCTGGCGCCCTG
CTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACC
AGCGGCGTGCACACCTTCCCGGCTGTCCTACAGTCCTCAGGACTCTCT3′

Amino acid sequence of heavy chain variable region and a portion of constant region encoded by SEQ ID NO:45:

(SEQ ID NO: 140)
XXXXEQSGGGVVQPGRSLRLSCAASGFTFSSYGMYWVRQAPGKGLEWVAV
IWYDGSNKYYADSVKGRFTISRDNSKNTLYLQMNSLRAEDTAVYYCARDF
YDSSRYHYGMDVWGQGTTVTVSSASTKGPSVFPLAPCSRSTSESTAALGC
LVKDYFPEPVTVSWNSGALTSGVHTFPAVLQSSGLS

Nucleotide sequence of light chain variable region and a portion of constant region:

(SEQ ID NO: 47)
5′ACTCAGTGTCCACTCTCCCTGCCCGTCACCCCTGGAGAGCCGGCCTCC
ATCTCCTGCAGGTCTAGTCAGAGCCTCTTGGATAGTGATGATGGAAACAC
CTATTTGGACTGGTACCTGCAGAAGCCAGGGCAGTCTCCACAGCTCCTGA
TCTATACGGTTTCCTATCGGGCCTCTGGAGTCCCAGACAGGTTCAGTGGC
AGTGGGTCAGGCACTGATTTCACACTGAAAATCAGCAGGGTGGAGGCTGA
GGATGTTGGAGTTTATTACTGCATGCAACGTATAGAGTTTCCGATCACCT
TCGGCCAAGGGACCCGACTGGAGATTAAACGAACTGTGGCTGCACCATCT
GTCTTCATCTTCCCGCCATCTGATGAGCAGTTGAAATCTGGAACTGCCTC
TGTTGTGTGCCTGCTGAATAA3′

Amino acid sequence of light chain variable region and a portion of constant region encoded by SEQ ID NO:47:

(SEQ ID NO: 141)
XXXXTQCPLSLPVTPGEPASISCRSSQSLLDSDDGNTYLDWYLQKPGQSP
QLLIYTVSYRASGVPDRFSGSGSGTDFTLKISRVEAEDVGVYYCMQRIEF
PITFGQGTRLEIKRTVAAPSVFIFPPSDEQLKSGTASVVCLLN

### Example 20

### In Vivo Studies Demonstrating Usefulness of Anti-Tim-1 Antibodies for the Treatment of Ovarian Cancer

An in vivo study was performed to assess the potency and therapeutic efficacy of the antibody-drug conjugate, CR014-vcMMAE, against an established human IGROV-1 ovarian xenograft in athymic mice.

### Materials and Methods:

Test Animals: Five- to 6-week old athymic mice (CD-1 nu/nu females), used for human tumor xenografts, were obtained from Charles Rivers Laboratories (Wilmington, Del.). Animals were housed in specific pathogen-free conditions, according to the guidelines of the Association for Assessment and Accreditation of Laboratory Animal Care International (AAALAC International). Test animals were provided pelleted food and water ad libitum and kept in a room with conditioned ventilation (HVAC), temperature (22°±2° C.), relative humidity (55%±15%), and photoperiod (12 hr). All studies were carried out with approved institutional animal care and use protocols. Contract Research Organizations. Experiments in vivo were conducted at Southern Research Institute (Birmingham, Ala.).

Human Ovarian Carcinoma Xenograft Model. The tumor inhibitory activity of the CR014-MMAE immunoconjugate was measured in an anti-tumor xenograft model using athymic mice, according to published methods (Geran R I, Greenberg N H, Macdonald M M, Schumacher A M and Abbott B J (1972) Protocols for screening chemical agents and natural products against animal tumors and other biological systems. Cancer Chemother Rep 3:1-104).

Briefly, test animals were implanted subcutaneously by trocar with small fragments of the IGROV1 carcinoma (30-60 mg) excised from athymic mouse tumor donors. When tumors became established (day 20, 95 mg), the animals were pair-matched into groups (n=6 mice/group), and treatment was administered by intravenous injection (tail vein).

The IGROV1 ovarian carcinoma was derived from a 47 yr. old woman in 1985, and was obtained from the American Type Culture Collection. The effects of treatment were monitored by repetitive tumor measurements across 2 diameters with Vernier calipers; tumor size (in mg) was calculated using a standard formula, (W²×L)/2, assuming a specific gravity of 1.0. Tumor size and body weights were assessed twice weekly. Mice were examined daily, however, and moribund animals were humanely euthanized if clinical indications of excessive pain or distress were noted (i.e., prostration, hunched posture, paralysis/paresis, distended abdomen, ulcerations, abscesses, seizures, and/or hemorrhages). Animals with tumors exceeding 2,000 mg were removed from the study and euthanized humanely.

Xenograft studies in the athymic mouse have been shown to effectively demonstrate anti-tumor effects for a variety of agents which have been found subsequently to have activity against clinical cancer Johnson J I, Decker S, Zaharevitz D, Rubinstein L V, Venditti J M, Schepartz S, Kalyandrug S, Christian M, Arbuck S, Hollingshead M and Sausville E A (2001) Relationships between drug activity in NCI preclinical in vitro and in vivo models and early clinical trials. Br J Cancer 84:1424-1431.

### Results:

Anti-Tumor Effects In Vivo vs. IGROV1. Based on the potency and cytotoxicity of CR014-vcMMAE against TIM-1-expressing cells in vitro, the anti-tumor effects were examined in vivo.

The effects of vehicle control groups, reference agents and the CR014-vcMMAE immunoconjugate on the growth of subcutaneous human IGROV1 ovarian carcinoma are shown in FIG. 20.

Tumors in animals treated with saline or PBS grew progressively until the tumor mass reached 2,000 mg at which time the animals were removed from the study and euthanized humanely. IGROV1 tumors have a high “take” rate in immunocompromised hosts (93%) and a very low rate of spontaneous regression (0%) (Dykes D J, Abbott B J, Mayo J G, Harrison Jr. S D, Laster Jr W R, Simpson-Herren L and Griswold Jr. DP (1992) Development of human tumor xenograft models for in vivo evaluation of new antitumor drugs, in Immunodeficient mice in Oncology, vol. 42 (Fiebig H H and Berger D Pe eds) pp 1-22, Contrib. Oncol. Basel, Karger).

Two known anti-tumor reference agents, vinblastine sulfate (i.v., 1.7 mg/kg, q4d×4) and paclitaxel (i.v., 24 mg/kg, q2d×4) were used in this study; these agents were administered at the maximum tolerated dose (MTD) determined in prior studies. Vinblastine produced a very slight, but not significant, anti-tumor effect (P≦0.20); Paclitaxel, however, showed significant tumor growth inhibition and produced complete regression of the ovarian tumors (n=6/6); re-growth of tumors was not observed during the observation period (i.e., 101 days after the commencement of treatment). Paclitaxel, but not vinblastine, has known efficacy in clinical ovarian carcinoma (Markman, M., Taxol: an important new drug in the management of epithelial ovarian cancer. Yale J Biol Med, 1991. 64(6): p. 583-90).

The anti-tumor effects of CR014-vcMMAE administered i.v. to IGROV1-bearing mice were remarkable. The CR014 immunoconjugate, when dosed at very high levels, however, produced lethal toxicity at 50 mg/kg/treatment (1/6=17%) and 100 mg/kg/treatment (6/6=100%). Nevertheless, 5/6 animals dosed at 50 mg/kg/treatment showed complete regression of the human ovarian carcinoma. Lower doses, such as 25, 12.5 and 6.25 mg/kg/treatment were therapeutically effective producing tumor growth inhibition which led to complete regressions for the majority of test animals. Tumors that regressed did not re-grow during the observation period.

The animals in this study (CR014-ONC-1, CGC-17) showed no abnormal treatment effects on gross examination at doses below 100 mg/kg; at 50 mg/kg inhibition of body weight and fatal toxicity occurred in only one of six mice. Below 50 mg/kg/treatment, twice weekly body weight determinations showed no observable or statistically significant effects of treatment with CR014-vcMMAE on body weight or weight gain.

### CONCLUSIONS

CR014-vcMMAE produces substantial, dose-dependent anti-tumor effects that began as tumor growth inhibition but soon led to complete regression of established human ovarian xenografts; the regressions were long-lived and re-growth of tumors after successful therapy was not been noted during the observation period (101 days after first day of treatment).

### INCORPORATION BY REFERENCE

All references cited herein, including patents, patent applications, papers, text books, and the like, and the references cited therein, to the extent that they are not already, are hereby incorporated herein by reference in their entirety. In addition, the following references are also incorporated by reference herein in their entirety, including the references cited in such references:

### EQUIVALENTS

While the preferred embodiment of the invention has been illustrated and described, it is to be understood that this invention is capable of variation and modification by those skilled in the art to which it pertains, and is therefore not limited to the precise terms set forth, but also such changes and alterations which may be made for adapting the invention to various usages and conditions. Accordingly, such changes and alterations are properly intended to be within the full range of equivalents, and therefore within the purview of the following claims.

The invention and the manner and a process of making and using it has been described in such full, clear, concise and exact terms so as to enable any person skilled in the art to which it pertains, or with which it is most nearly connected, to make and use the same.

What is claimed is:

1. A method of effectively treating ovarian cancer comprising administering to a patient in need thereof a therapeutically effective dose of an antibody or binding fragment thereof, that specifically binds to T cell, immunoglobulin domain or mucin domain 1 (TIM-1).

2. The method of claim 1, wherein said antibody comprises the amino acid sequence shown in SEQ ID NO:54.

3. The method of claim 1, wherein said antibody is a monoclonal antibody.

4. The method of claim 1, wherein said antibody binds to TIM-1 with a Kd between 10⁻⁷ and 10⁻¹⁴ M.

5. The method of claim 1, wherein said antibody or binding fragment is conjugated to a therapeutic agent.

6. The method of claim 5, wherein said therapeutic agent is a toxin.

7. The method of claim 5, wherein said therapeutic agent is a radioactive isotope.

8. The method of claim 5, wherein said therapeutic agent is a chemotherapeutic agent.

9. A method of effectively treating renal cancer comprising administering to a patient in need thereof a therapeutically effective dose of an antibody or binding fragment thereof, that specifically binds to T cell, immunoglobulin domain or mucin domain 1 (TIM-1).

10. The method of claim 9, wherein said antibody comprises the amino acid sequence shown in SEQ ID NO:54.

11. The method of claim 9, wherein said antibody is a monoclonal antibody.

12. The method of claim 9, wherein said antibody binds to TIM-1 with a Kd between 10⁻⁷ and 10⁻¹⁴ M.

13. The method of claim 9, wherein said antibody or binding fragment is conjugated to a therapeutic agent.

14. The method of claim 13, wherein said therapeutic agent is a toxin.

15. The method of claim 13, wherein said therapeutic agent is a radioactive isotope.

16. The method of claim 13, wherein said therapeutic agent is a chemotherapeutic agent.
